(19) United States
(12) Patent Application Publication
(10) Pub. No.: US 2024/0096447 A1
(43) Pub. Date: Mar. 21, 2024

(54) METHODS OF GENERATING PHENOCOPY SIGNATURES AND USES THEREOF

(71) Applicant: Wisconsin Alumni Research Foundation, Madison, WI (US)

(72) Inventors: Shuang Zhao, Verona, WI (US); Hamza Bakhtiar, Mequon, WI (US)

(73) Assignee: Wisconsin Alumni Research Foundation, Madison, WI (US)

(21) Appl. No.: 18/522,977

(22) Filed: Nov. 29, 2023

Related U.S. Application Data
(63) Continuation-in-part of application No. 18/326,364, filed on May 31, 2023.
(60) Provisional application No. 63/347,300, filed on May 31, 2022.

(51) Int. Cl. G16B 25/10; G16H 20/10

(52) U.S. Cl. CPC G16B 25/10; G16H 20/10

(57) ABSTRACT

Methods of generating phenocopy signatures and uses thereof. The methods of generating phenocopy signatures can include determining gene expression signatures that predict the presence of mutations in training cells. Uses of the phenocopy signatures include identifying cells exhibiting the phenocopy signatures, identifying subjects comprising cells that exhibit the phenocopy signatures, methods of using the phenocopy signatures to predict cells sensitive to treatment with drugs, methods of treating cells with phenocopy signatures predicted to be sensitive to treatment with drugs, methods of using phenocopy signatures to predict subjects sensitive to treatment with drugs, and methods of treating subjects with phenocopy signatures predicted to be sensitive to treatment with drugs.

### CROSS-REFERENCE TO RELATED APPLICATIONS

The present application is a continuation-in-part of U.S. application Ser. No. 18/326,364, filed May 31, 2023, which claims priority to Provisional U.S. application Ser. No. 63/347,300, filed May 31, 2022, all of which are incorporated herein by reference in their entireties.

### FIELD OF THE INVENTION

The invention is directed to methods of generating phenocopy signatures and uses thereof, including identifying cells exhibiting phenocopy signatures, identifying subjects comprising cells that exhibit phenocopy signatures, methods of using phenocopy signatures to predict cells sensitive to treatment with drugs, methods of treating cells with phenocopy signatures predicted to be sensitive to treatment with drugs, methods of using phenocopy signatures to predict subjects sensitive to treatment with drugs, and methods of treating subjects with phenocopy signatures predicted to be sensitive to treatment with drugs.

### BACKGROUND

DNA mutations in specific genes can confer preferential benefit from drugs targeting those genes. However, other molecular perturbations can “phenocopy” pathogenic mutations, but would not be identified using standard clinical sequencing, leading to missed opportunities for other patients to benefit from targeted treatments.

Methods for determining phenocopy signatures of mutations in genes that are useful for predicting efficacy of targeted drugs regardless of mutation status are needed.

### SUMMARY OF THE INVENTION

One aspect of the invention is directed to methods of generating phenocopy signatures. The methods can comprise identifying a gene set comprising one or more genes within a biological pathway, identifying a set of training cells, wherein each training cell comprises each of the one or more genes in the gene set, obtaining a nucleic acid sequence for each of the one or more genes in each training cell, identifying a mutation set comprising one or more mutations within the nucleic acid sequences, obtaining a gene expression profile for each training cell, and determining from the mutation set and the gene expression profiles a set of gene expression signatures that predict presence of the one or more mutations within the training cells. The phenocopy signature thereby comprises the set of gene expression signatures.

Another aspect of the invention is directed to methods of identifying cells exhibiting a phenocopy signature. The methods can comprise obtaining a gene expression profile for a test cell and determining whether the gene expression profile for the test cell matches the phenocopy signature. The test cell exhibits the phenocopy signature if the gene expression profile for the test cell matches the phenocopy signature.

Another aspect of the invention is directed to methods of predicting cells sensitive to treatment with drugs that target a biological pathway and, optionally, treating the cell. The methods can comprise obtaining a gene expression profile for a test cell, and determining whether the gene expression profile for the test cell matches a phenocopy signature generated with a gene set from the biological pathway that the drug targets. The cell is predicted to be sensitive to treatment with the drug if the gene expression profile for the test cell matches the phenocopy signature. The methods can further comprise administering the drug to the cell if the cell is predicted to be sensitive to treatment with the drug.

Another aspect of the invention is directed to methods of identifying a subject comprising cells that exhibit the phenocopy signature. The methods can comprise isolating a test cell from the subject, obtaining a gene expression profile for the test cell, and determining whether the gene expression profile for the test cell matches the phenocopy signature, wherein the test cell exhibits the phenocopy signature if the gene expression profile for the test cell matches the phenocopy signature.

Another aspect of the invention is directed to methods of predicting subjects sensitive to treatment with drugs that target a biological pathway and, optionally, treating the subject with the drug. The methods can comprise obtaining a gene expression profile for a test cell isolated from the subject and determining whether the gene expression profile for the test cell matches a phenocopy signature generated with a gene set from the biological pathway that the drug targets. The subject is predicted to be sensitive to treatment with the drug if the gene expression profile for the test cell matches the phenocopy signature. The subject can then be administered the drug to the subject if the subject is predicted to be sensitive to treatment with the drug.

The objects and advantages of the invention will appear more fully from the following detailed description of the preferred embodiment of the invention made in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Schematic of phenocopy signatures model. For each gene of interest, known mutation status is determined (green—mutation; purple—no mutation) and an XGBoost model is trained on mutation status based on gene expression of the pathway of interest, and the phenocopy signatures are then locked (Training, left). Next, gene expression data from cell line databases or clinical studies are input into the phenocopy signatures which outputs a predicted phenocopy status. This phenocopy status is then compared to known DNA mutations for predicting drug response (Validation, right).

FIG. 2. Phenocopy signature predictions versus DNA mutations. Venn diagrams depict the number of cell lines assigned as phenocopies (left circles) compared to actual DNA mutations across the eight pathways tested (right circles). DNA mutations are further divided into those that are annotated as pathogenic by ClinVar or various computational tools (tops of right circles) and those that have unknown significance (bottoms of right circles).

FIG. 3. Phenocopy signatures significantly add to DNA mutations in predicting drug response across oncogenic pathways. Linear models for drug response were used to assess how much the phenocopy signatures added to DNA mutations across pathways. Each data point used in the boxplot represents a model for a single drug in a dataset. A larger chi-squared value represents a more significant contribution of the phenocopy signature to DNA mutations. Dotted line represents a significant FDR threshold of 0.05.

FIGS. 4A-4H. Detailed comparison of phenocopy signatures to DNA mutations. Linear models for drug response were used to assess how much the phenocopy signatures added to DNA mutations across pathways. Each model of a single drug is represented by three points, one for each independent variable (DNA mutation, pathogenic mutation, and phenocopy signature). The x-axis represents the linear coefficient, and the y-axis is the associated −Log₁₀(p-value) of each independent variable in each linear model. Pathogenic mutations are mutations which are annotated as pathogenic by ClinVar or computational tools. Negative coefficients represent expected estimates, where the actual mutation status or predicted mutation status from the phenocopy signature is associated with increased sensitivity to the drug. Data points in the upper-left quadrant therefore represent drugs for which the phenocopy signature most significantly contributed to predicting drug sensitivity. Data are shown for the following pathways: EGFR (FIG. 4A); BRAF (FIG. 4B); PI3K-AKT (FIG. 4C); PARP/HRD (FIG. 4D); MAPK (FIG. 4E); ERBB2 (FIG. 4F); MTOR (FIG. 4G); and JAK (FIG. 4H).

FIGS. 5A-5H. Sensitivity and specificity of phenocopy signatures. Sensitivity and specificity were calculated for each combination of drug and pathway. The top quartile in drug sensitivity for each drug was considered a responder. Each data point represents a single drug in a single dataset, with the three datasets represented by different shapes. Five separate conditions were investigated: (1) Phenocopy signature in cancer cell lines without DNA mutations, (2) Phenocopy signature in cancer cell lines with pathogenic DNA mutations, (3) Phenocopy signature in cancer cell lines with non-pathogenic DNA mutations, (4) DNA mutations in all cancer cell lines, and (5) Pathogenic DNA mutations in all cancer cell lines. Data are shown for the following pathways: EGFR (FIG. 5A); BRAF (FIG. 5B); PI3K-AKT (FIG. 5C); PARP/HRD (FIG. 5D); MAPK (FIG. 5E); ERBB2 (FIG. 5F); MTOR (FIG. 5G); and JAK (FIG. 5H).

FIGS. 6A-6H. Positive predictive value (PPV) and negative predictive value (NPV) of phenocopy signatures. PPV and NPV were calculated for each drug and pathway. The top quartile in drug sensitivity for each drug was considered a responder. Each data point represents a single drug in a single dataset, with the three datasets represented by different shapes. Five separate conditions were investigated: (1) Phenocopy signature in cancer cell lines without DNA mutations, (2) Phenocopy signature in cancer cell lines with pathogenic DNA mutations, (3) Phenocopy signature in cancer cell lines with non-pathogenic DNA mutations, (4) DNA mutations in all cancer cell lines, and (5) Pathogenic DNA mutations in all cancer cell lines. Data are shown for the following pathways: EGFR (FIG. 6A); BRAF (FIG. 6B); PI3K-AKT (FIG. 6C); PARP/HRD (FIG. 6D); MAPK (FIG. 6E); ERBB2 (FIG. 6F); MTOR (FIG. 6G); and JAK (FIG. 6H).

FIG. 7. Clinical validation of phenocopy signatures. BRAF and mTOR phenocopy signatures were applied to BRAF-mutant melanoma (A) and breast cancer cohorts (B-C), respectively. Altered or unaltered status indicates the alteration status assigned by the BRAF/mTOR phenocopy signatures. Pre-treatment samples were considered sensitive, and post-treatment samples were considered resistant per the original datasets.

FIG. 8. Schematic of TP53 loss phenocopy signature model development. In the TCGA training cohort, TP53 loss genomic status (purple—genomic TP53 loss sample, green—no genomic TP53 loss sample) and expression of p53-pathway relevant genes are utilized to generate a TP53 loss phenocopy transcriptional signature using an XGBoost machine learning model (Training, left). To validate this model, the TP53 loss phenocopy signature is then used to predict phenocopy status from expression data in cell line databases (GDSC, CCLE, DepMap) and predicted phenocopy status (phenocopy versus not) is compared to genomic TP53 loss across cancer cell lines for predicting chemotherapy response (Independent

Validation, left). Finally, phenocopy status is predicted from expression data across the breast cancer clinical cohorts and association with neoadjuvant chemotherapy response is evaluated (Independent Validation, left).

FIGS. 9A-9C. Association between DNA mutation status and TP53 phenocopy prediction in training and in vitro validation cohorts. (FIG. 9A) In the TCGA training cohort (n=9,428), significantly more TP53 mutated samples were predicted p53 loss phenocopies compared to TP53 non-mutated samples (81% vs 55%, p<0.0001). (FIG. 9B) In the GDSC in vitro validation cohort (n=950), 66.7% of TP53 mutated vs 54.5% TP53 non-mutated samples were predicted p53 loss phenocopies (p=0.0178). (FIG. 9C) in the CCLE/DepMap in vitro validation cohort (n=917), 70.9% of TP53 mutated vs 50.6% TP53 non-mutated samples were predicted p53 loss phenocopies (p<0.0001).

FIG. 10. TP53 phenocopy signature predicts chemotherapy response in vitro. Linear models for cytotoxic chemotherapy response were used to assess how much the RNA-based TP53 loss phenocopy signature added to DNA-based genomic TP53 loss in the cell line datasets. Each model of a single chemotherapy is represented by two points, one for each independent variable (genomic TP53 loss in green, TP53 loss phenocopy signature in red). The x-axis represents the linear coefficient, and the y-axis is the associated -Log10(p-value) of each independent variable in the linear model. Negative coefficients represent expected estimates, where the genomic or phenocopy TP53 loss status is associated with increased sensitivity to each chemotherapy. Data points in the upper-left quadrant therefore represent drugs for which the phenocopy signature most significantly contributed to predicting chemotherapy sensitivity.

FIGS. 11A-11C. TP53 loss phenocopy signature is associated with neoadjuvant chemotherapy response. (FIG. 11A) TP53 phenocopy status is significantly associated with pathologic complete response to neoadjuvant chemotherapy in a combined cohort of 3,011 early-stage breast cancers (p<0.0001). This association is seen in both ER positive (FIG. 11B) and ER negative (FIG. 11C) breast cancers.

FIGS. 12A and 12B. TP53 phenocopy signature is associated with residual cancer burden in the BrighTNess trial and decreases during neoadjuvant chemotherapy in the I-SPY1 trial (FIG. 12A) TP53 phenocopy status is significantly associated with RCB class after neoadjuvant therapy in the phase III BrighTNess clinical trial in triple negative breast cancer (n=407, p=0.00518). (FIG. 12B) In serial tissue samples before, during and after neoadjuvant therapy in the I-SPY1 clinical trial, TP53 phenocopy proportion decreases over the course of neoadjuvant therapy (p=0.0273).

FIGS. 13A-C. TP53 phenocopy signature is associated with neoadjuvant chemoimmunotherapy response in the I-SPY2 trial. (FIG. 13A) Validation study demonstrating that TP53 phenocopy status is significantly associated with pathologic complete response to anthracycline/taxane-based chemotherapy across all arms of the I-SPY2 trial (n=988, p=0.00024). Compared to standard anthracycline/taxane chemotherapy (FIG. 13B), phenocopy status is associated with a particularly high rate of pathologic complete response to chemoimmunotherapy (FIG. 13C).

### DETAILED DESCRIPTION OF THE INVENTION

One aspect of the invention is directed to methods of generating a phenocopy signature. The methods of generating a phenocopy signature can comprise identifying a gene set comprising one or more genes within a biological pathway.

As used herein, “biological pathway” refers to any network of interactions and reactions among molecules in a cell that leads to a certain product or a change in a cell, tissue, or organism. Biological pathways can result in the assembly of molecules, such as lipids or proteins, cause changes in tissues, turn genes on or off, transmit a signal, or induce any other change in a cell, tissue, or organism. The molecules participating in a biological pathway are sometimes referred to in the art as “entities.” The molecules (or entities) can include nucleic acids (e.g., DNA, including subparts thereof such as genes or other genetic elements; RNA, including RNA genes, mRNA, microRNA etc.), proteins (e.g., signaling proteins, enzymes, structural proteins, etc.), small molecules, carbohydrates (e.g., monosaccharides, oligosaccharides, polysaccharides, lipids (e.g., cholesterol, fatty acids, fatty acid esters, etc.), and polymers (e.g., collagen, etc.), or any other molecule participating in a biological reaction. Exemplary types of pathways include cell cycle pathways, DNA repair pathways, metabolism pathways, signaling pathways (e.g., signal transduction by a receptor (e.g., a growth factor receptor) and second messengers), transcriptional regulation pathways, transport pathways (e.g., of transmembrane transporters), cell motility pathways, immune function pathways, cell death pathways, host-virus interaction pathways, cellular stress-response pathways, developmental pathways, senescence pathways, angiogenesis pathways, epithelial-to-mesenchymal transition pathways, and neural pathways, among others.

Methods for identifying specific pathways and their constituent molecules (including genes), interactions, and reactions are well known in the art. A large number of types of biological pathways and particular biological pathways, including their constituent molecules (including genes), interactions, and reactions, are annotated in public databases. A database employed in following examples is the Reactome database. See reactome.org and Fabregat et al. 2018 (Fabregat A, Jupe S, Matthews L, Sidiropoulos K, Gillespie M, Garapati P, Haw R, Jassal B, Korninger F, May B, Milacic M, Roca C D, Rothfels K, Sevilla C, Shamovsky V, Shorser S, Varusai T, Viteri G, Weiser J, Wu G, Stein L, Hermjakob H, D'Eustachio P. The Reactome Pathway Knowledgebase. Nucleic Acids Res. 2018 Jan. 4; 46(D1):D649-D655). Reactome is an open-source, open access, manually curated and peer-reviewed biological pathway database. It provides tools for the visualization, interpretation, and analysis of biological pathways. All data and software in the database are freely available for download. Interaction, reaction, and pathway data are provided as downloadable flat, Neo4j GraphDB, MySQL, BioPAX, SBML and PSI-MITAB files and are also accessible through Reactome's web services application programming interfaces. Software and instructions for local installation of the Reactome database, website, and data entry tools are also available to support independent pathway curation. Other databases include KEGG (www.kegg.jp) (Kanehisa M, Goto S. KEGG: kyoto encyclopedia of genes and genomes. Nucleic Acids Res. 2000 Jan. 1; 28(1):27-30), WikiPathways (www.wikipathways.org) (Pico A R, Kelder T, van Iersel M P, Hanspers K, Conklin BR, Evelo C. WikiPathways: pathway editing for the people. PLoS Biol. 2008 Jul. 22; 6(7):e184), NCI-Nature Pathway Interaction Database (www.ndexbio.org) (Schaefer C F, Anthony K, Krupa S, Buchoff J, Day M, Hannay T, Buetow K H. PID: the Pathway Interaction Database. Nucleic Acids Res. 2009 January; 37(Database issue):D674-9), PhosphoSitePlus (www.phosphosite.org) (Hornbeck P V, Zhang B, Murray B, Kornhauser J M, Latham V, Skrzypek E. PhosphoSitePlus, 2014: mutations, PTMs and recalibrations. Nucleic Acids Res. 2015 January; 43(Database issue):D512-20), BioCyc (biocyc.org) (Caspi R, Altman T, Dreher K, Fulcher C A, Subhraveti P, Keseler I M, Kothari A, Krummenacker M, Latendresse M, Mueller L A, Ong Q, Paley S, Pujar A, Shearer A G, Travers M, Weerasinghe D, Zhang P, Karp P D. The MetaCyc database of metabolic pathways and enzymes and the BioCyc collection of pathway/genome databases. Nucleic Acids Res. 2012 January; 40(Database issue):D742-53), PANTHER (Protein ANalysis THrough Evolutionary Relationships) (pantherdb.org) (Thomas P D, Kejariwal A, Campbell M J, Mi H, Diemer K, Guo N, Ladunga I, Ulitsky-Lazareva B, Muruganuj an A, Rabkin S, Vandergriff J A, Doremieux O. PANTHER: a browsable database of gene products organized by biological function, using curated protein family and subfamily classification. Nucleic Acids Res. 2003 Jan. 1; 31(1):334-41), TRANSFAC (TRANScription FACtor database) (gene-regulation.com) (Wingender E. The TRANSFAC project as an example of framework technology that supports the analysis of genomic regulation. Brief Bioinform. 2008 July;9(4):326-32), DrugBank (www.drugbank.com) (Wishart D S, Knox C, Guo A C, et al. DrugBank: a comprehensive resource for in silico drug discovery and exploration. Nucleic Acids Res. 2006;34(Database issue):D668-D672), esyN (www.esyn.org/) (Bean D M, Heimbach J, Ficorella L, Micklem G, Oliver S G, Favrin G. esyN: network building, sharing and publishing. PLoS One. 2014 Sep 2;9(9):e106035), Comparative Toxicogenomics Database (CTD) (ctdbase.org) (Mattingly C J, Rosenstein M C, Colby G T, Forrest J N Jr, Boyer J L. The Comparative Toxicogenomics Database (CTD): a resource for comparative toxicological studies. J Exp Zool A Comp Exp Biol. 2006 Sep. 1; 305(9):689-92), and Pathway Commons (www.pathwaycommons.org/) (Cerami E G, Gross B E, Demir E, Rodchenkov I, Babur O, Anwar N, Schultz N, Bader G D, Sander C. Pathway Commons, a web resource for biological pathway data. Nucleic Acids Res. 2011 January; 39(Database issue) :D685-90), among others.

In some versions, the biological pathway is a disease pathway. A disease pathway is a biological pathway whose activity produces, causes, or contributes to a disease or any symptom thereof. Exemplary disease pathways include signal transduction pathways by growth factor receptors and second messengers, mitotic cell cycle pathways, cellular stress-response pathways, programmed cell death pathways, DNA repair pathways, transmembrane transporter pathways, metabolism pathways, infectious disease pathways, immune system pathways, neuronal system pathways, developmental biology pathways, and hemostasis pathways, among others. Exemplary diseases resulting from disease pathways include cancer, such as colorectal cancer, pancreatic cancer, hepatocellular carcinoma, gastric cancer, glioma, thyroid cancer, acute myeloid leukemia, chronic myeloid leukemia, basal cell carcinoma, melanoma, renal cell carcinoma, bladder cancer, prostate cancer, endometrial cancer, breast cancer, small cell lung cancer, and non-small cell lung cancer, among others. Exemplary diseases resulting from disease pathways include immune disease, such as asthma, systemic lupus erythematosus, rheumatoid arthritis, autoimmune thyroid disease, inflammatory bowel disease, allograft rejection, graft-versus-host disease, and primary immunodeficiency. Exemplary diseases resulting from disease pathways include neurodegenerative disease, such as Alzheimer's disease, Parkinson's disease, amyotrophic lateral sclerosis, Huntington's disease, spinocerebellar ataxia, prion disease, and neurodegeneration diseases, among others. Exemplary diseases resulting from disease pathways include substance dependence, such as cocaine addiction, amphetamine addiction, morphine addiction, nicotine addiction, and alcoholism, among others. Exemplary diseases resulting from disease pathways include cardiovascular disease, such as hyperlipidemia, atherosclerosis, hypertrophic cardiomyopathy, arrhythmogenic right ventricular cardiomyopathy, dilated cardiomyopathy, diabetic cardiomyopathy, and viral myocarditis, among others. Exemplary diseases resulting from disease pathways include endocrine and metabolic diseases, such as type II diabetes mellitus, type I diabetes mellitus, maturity onset diabetes of the young, alcoholic liver disease, non-alcoholic fatty liver disease, insulin resistance, and Cushing syndrome, among others. Exemplary diseases resulting from disease pathways include antimicrobial drug resistance, including beta-lactam resistance, vancomycin resistance, and cationic antimicrobial peptide (CAMP) resistance, among others. Exemplary diseases resulting from disease pathways include antineoplastic drug resistance, including EGFR tyrosine kinase inhibitor resistance, platinum drug resistance, antifolate resistance, and endocrine resistance, among others. In some versions, the disease pathway is a cancer pathway. A cancer pathway is a disease pathway whose atypical activity produces cancer. Various disease pathways, including cancer pathways, are described in the biological pathway databases described herein. Constituent genes (and corresponding proteins and other molecules in the pathway), interactions, and reactions in disease pathways can be determined using methods known in the art and/or the biological pathway databases described herein.

The one or more genes within the identified gene set can include any number of genes. In various versions of the invention, the one or more genes can be fewer than 50, fewer than 40, fewer than 30, fewer than 20, fewer than 15, fewer than 10, fewer than 9, fewer than 8, fewer than 7, fewer than 6, fewer than 5 fewer than 4, fewer than 3, fewer than 2, or exactly 1.

In preferred versions of the invention, the one or more genes within the identified gene set comprises at least one key driver gene of a disease. “Key driver gene” is used in this context to refer to a gene whose normal or abnormal functions produce, cause, or contribute to a disease or any symptom thereof. In some versions, a key driver gene is a gene that, when mutated, produces, causes, or contributes to a disease or any symptom thereof.

In some versions of the invention, the one or more genes within the identified gene set comprises at least one gene for which there is at least one allele within the population comprising a mutation that is a disease biomarker. In some versions, the disease biomarker is a US Food and Drug Administration (FDA)-qualified disease biomarker. “Disease biomarker” used with reference to a mutation refers to a mutation that indicates a given disease or an increased likelihood of developing the given disease.

In some versions of the invention, the one or more genes within the identified gene set comprises at least one gene for which there is at least one allele within the population comprising a mutation that is a disease treatment biomarker. In some versions, the disease treatment biomarker is a US Food and Drug Administration (FDA)-qualified disease treatment biomarker. “Disease treatment biomarker” used with reference to a mutation refers to a mutation that indicates treatability of a particular disease with a particular drug.

In some versions of the invention, the one or more genes within the identified gene set are comprised by a disease pathway of a disease that is treatable with a drug. “Treatable with a drug” as used herein refers to the ability of the drug to provide any degree of amelioration of the disease or symptom of the disease in at least a subset of individuals who have the disease. “Disease of a disease pathway,” refers herein to a disease associated with, affected by, resulting from, caused by, or enhanced by a particular disease pathway. “Drug” as used herein refers to any active agent that can induce a physiological change in a cell, tissue, or organism.

The methods of generating a phenocopy signature can comprise identifying a set of training cells. Each training cell preferably comprises the biological pathway(s) in which the one or more genes in the gene set is or are comprised. Each training cell preferably comprises each of the one or more genes in the gene set. The number of training cells in the gene set can include any number of individual cells, types of cells, or samples of cells.

The training cells in the set can comprise pathological cells, healthy cells, or a combination thereof. In some versions, at least one or more of the training cells comprises a pathological cell. In some versions, each of the training cells is a pathological cell.

“Pathological cell” refers to a cell that has at least one structural or functional abnormality with regard to a healthy cell. Distinctions between pathological cells and healthy cells are well-known in the art of pathology. Any pathological cell can be a “physiological pathological cell,” which is a pathological cell obtained or derived from an organism, or a “model pathological cell”, which is a model cell that models a pathological cell obtained or derived from an organism. One or more of the pathological cells comprised by the training cells can be a pathological cell of a particular disease. “Pathological cell of a disease,” and variants thereof, refers herein to a cell associated with, resulting from, causing, or contributing to a particular disease. Exemplary training cells provided herein include cancer cells. However, pathological cells from any other type of disease can be employed.

The methods of generating a phenocopy signature can comprise obtaining a nucleic acid sequence for at least one, some, or each of the one or more genes in each training cell. The nucleic acid sequence can include the entire gene sequence or any one or more sub-portions thereof, such as exons, introns, and/or mutational “hotspots,” etc. Obtaining the nucleic acid sequence can comprise sequencing at least a portion of one or more the genes, downloading such sequences from a database, or other methods. Sequencing the genes can comprise sequencing the gene (or sub-portion thereof), sequencing mRNA from the gene (or sub-portion thereof) and deducing the gene sequence from the mRNA sequence, or other methods.

The methods of generating a phenocopy signature can comprise identifying a mutation set comprising one or more mutations within the nucleic acid sequences. In some versions, the mutations can be determined with respect to a germline sequences of an individual subject. The individual subject can be a subject from which one or more training cells are obtained or derived. In some versions, particularly when germline sequences of an individual subject are not available, the mutations can be determined with respect to a reference genome. The mutations can include any perturbation in the genome, including copy number variants, structural variants, gene fusions, point mutations, insertions, deletions, substitutions, or any other difference between a given nucleic acid sequence and a reference sequence. The mutations can comprise non-coding mutations, coding mutations, or a combination thereof. In exemplary versions of the invention, the mutations in the mutation set consist of coding mutations. The mutations can comprise pathogenic mutations, non-pathogenic mutations, or a combination thereof “Pathogenic mutations” are mutations that are known or predicted to be pathogenic, i.e., increase an individual's susceptibility or predisposition to a certain disease or disorder. Pathogenic mutations can be predicted with a number of computational tools and methods, such as ClinVar (www.ncbi.nlm.nih.gov/clinvar/) (Sethi A, Tully R, Villamarin-Salomon R, Rubinstein W, Maglott DR. ClinVar: public archive of interpretations of clinically relevant variants. Nucleic Acids Res. 2016 Jan. 4;44(D1):D862-8), SIFT (sift.bii.a-star.edu.sg) (Vaser R, Adusumalli S, Leng S N, Sikic M, Ng P C. SIFT missense predictions for genomes. Nat Protoc. 2016 January; 11(1): 1 -9), Polyphen-2 HVAR (genetics.bwh.harvard.edu/pph2) (Adzhubei I, Jordan D M, Sunyaev S R. Predicting functional effect of human missense mutations using PolyPhen-2. Curr Protoc Hum Genet. 2013 January; Chapter 7:Unit7.20), Polyphen-2 HDIV (genetics.bwh.harvard.edu/pph2) (Adzhubei I, Jordan D M, Sunyaev S R. Predicting functional effect of human missense mutations using PolyPhen-2. Curr Protoc Hum Genet. 2013 January; Chapter 7:Unit7.20), and FATHMM (fathmm.biocompute.org.uk) (Rogers M F, Shihab H A, Mort M, Cooper D N, Gaunt T R, Campbell C. FATHMM-XF: accurate prediction of pathogenic point mutations via extended features. Bioinformatics. 2018 Feb. 1;34(3):511-513). In exemplary versions of the invention, the mutations in the mutation set consist of pathogenic mutations. In some versions of the invention, the mutations in the mutation set are all the pathogenic coding mutations within the nucleic acid sequences. In some versions of the invention, at least one of the one or more mutations in the mutation set is a pathogenic mutation not known to be pathogenic, i.e., it is merely predicted to be pathogenic. In some versions of the invention, at least one of the one or more mutations in the mutation set is not a known disease biomarker or a known disease treatment biomarker.

The methods of generating a phenocopy signature can comprise obtaining a gene expression profile for at least one, some, or each training cell. As used herein, “gene expression profile” refers to a quantitation of the mRNA levels of different mRNA species present in a cell. “mRNA species” refers to a type of mRNA in a population of mRNA having at least one difference with respect to at least one other type of mRNA in the population of mRNA. In some versions, the mRNA species are defined by having different sequences with respect to each other. In some versions, the mRNA species are defined by virtue of corresponding to (e.g., being expressed from) different genes, which can be determined, for example, by the mRNA comprising a sequence that corresponds to a particular gene sequence. In some versions, the gene expression profile for any cell described herein comprises a quantitation of the mRNA levels of each mRNA species present in the cell. In some versions, the gene expression profile for any cell described herein comprises a quantitation of the mRNA levels of each mRNA species of each gene in the biological pathway present in the cell. In some versions, the gene expression profile for any cell described herein excludes a quantitation of the mRNA levels of at least one mRNA species of a gene not in the biological pathway present in the cell. Obtaining the gene expression profiles can comprise measuring the mRNA levels (e.g., measuring the number of mRNA copies) of different mRNA species present in the cells and/or, depending on the availability of data for a particular training cell, downloading the gene expression profiles from a database, among other methods. In some versions, obtaining the gene expression profiles can comprise measuring mRNA levels of one or more of the genes in the biological pathway. In some versions, obtaining the gene expression profiles can comprise measuring mRNA levels of each of the genes in the biological pathway. In some versions, obtaining the gene expression profiles exclude measuring mRNA levels of one or more genes not in the biological pathway. The mRNA levels can include raw numbers of measured mRNA copies or can include normalized and/or analytically processed values.

Methods for measuring the mRNA levels of different mRNA species present in cells are well known in the art, and include RNA-seq, among other methods. See Stark et al. 2019 (Stark R, Grzelak M, Hadfield J. RNA sequencing: the teenage years. Nat Rev Genet. 2019 November; 20(11):631-656).

The methods of generating a phenocopy signature can comprise determining from the mutation set and the gene expression profiles a set of gene expression signatures that predict the presence of at least a subset of the one or more mutations within the training cells. “Gene expression signature” as used herein refers to a particular combination of mRNA levels or ranges of mRNA levels for at least a subset of the mRNA species quantitated in the gene expression profiles. The set of such gene expression signatures can include any number of individual gene expression signatures. In some versions, the gene expression signatures include gene expression signatures of one or more of the genes in the biological pathway. In some versions, the gene expression signatures include gene expression signatures of each of the genes in the biological pathway. In some versions, the gene expression signatures exclude gene expression signatures of one or more of genes not in the biological pathway. Determining gene expression signatures for only a subset of genes in the genome can decrease “noise” in the data and increase processing time. The subset of the one or more mutations within the training cells predicted by the set of gene expression signatures can be any proportion of the one or mutations in the set. In various versions, the subset of the one or more mutations within the training cells predicted by the set of gene expression signatures comprises at least 5%, at least at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100% (by number) of the one or mutations in the set. (“Subset” in this context can encompass the entire set or any proportion thereof.) In various versions, the subset of the one or more mutations within the training cells predicted by the set of gene expression signatures comprises fewer than 50%, fewer than 55%, fewer than 60%, fewer than 65%, fewer than 70%, fewer than 75%, fewer than 80%, fewer than 85%, fewer than 90%, fewer than 95%, fewer than 100% (by number) of the one or more mutations in the set. Each mRNA level in the gene expression signatures can independently be a discrete value or a range of values. In some versions, each mRNA level in the gene expression signatures is a discrete value. The gene expression signatures that predict the presence of the one or more mutations can be determined using any of a variety of approaches. In exemplary versions of the invention, a gradient tree boosting approach is used to train phenocopy signatures that predict mutation status based on the RNA expression in the gene expression profiles. Any other machine learning technique used in regression and/or classification tasks can alternatively be used. Examples include support vector machine, random forest, elastic net regression (elastic net regularization), linear regression, k-nearest neighbor, ridge regression, lasso regression, neural networks, and Bayesian networks, among others.

In some versions of the invention, determining the phenocopy signature does not comprise incorporating empirical drug-response data for at least one of the training cells. In some versions of the invention, determining the phenocopy signature does not comprise incorporating empirical drug-response data for some of the training cells. In some versions of the invention, determining the phenocopy signature does not comprise incorporating empirical drug-response data for any of the training cells. In other words, the phenocopy signature is determined independently of any drug-response data for the training cells.

Another aspect of the invention is directed to methods of identifying cells exhibiting a phenocopy signature.

The methods of identifying cells exhibiting a phenocopy signature can comprise obtaining a gene expression profile for the test cell. Obtaining the gene expression profiles can comprise measuring mRNA levels of different mRNA species present in the test cell and/or, depending on the availability of data for a particular test cell, downloading the gene expression profiles from a database, among other methods. In some versions, obtaining the gene expression profiles can comprise measuring mRNA levels of one or more of the genes in the biological pathway. In some versions, obtaining the gene expression profiles can comprise measuring mRNA levels of each of the genes in the biological pathway. In some versions, obtaining the gene expression profiles exclude measuring mRNA levels of one or more genes not in the biological pathway. The test cell can be derived from any source, as described elsewhere herein.

The methods of identifying cells exhibiting a phenocopy signature can comprise determining whether the gene expression profile for the test cell matches the phenocopy signature. The test cell exhibits the phenocopy signature if the gene expression profile for the test cell matches the phenocopy signature. “Matching,” as used herein with respect to a gene expression profile matching a phenocopy signature, refers to the gene expression profile having a combination of mRNA levels that reside within defined ranges encompassing a particular combination of mRNA levels in at least one gene expression signature of the phenocopy signature. The defined ranges can be determined in any manner suitable for a particular purpose and can be informed by statistical or algorithmic analysis and confidence thresholds for that purpose. Defined ranges in the following examples are determined by a XGBoost model. Any other type of machine learning algorithm can be used to determine suitable ranges.

The methods of the invention are capable of identifying test cells exhibiting a phenocopy signature, even if the test cells themselves do not comprise any of the mutations used to generate the phenocopy signature, or any other known mutations. Accordingly, in some versions, the test cell comprises each of the one or more genes in the gene set and none of the one or more genes in the gene set of the test cell comprises any of the one or more mutations in the mutation set used to generate the phenocopy signature. In some versions, the test cell comprises each of the one or more genes in the gene set and none of the one or more genes in the gene set of the test cell comprises any mutation that is pathogenic or predicted to be pathogenic. In some versions, the test cell comprises each of the one or more genes in the gene set and none of the one or more genes in the gene set of the test cell comprises any coding mutation. In some versions, the test cell comprises each of the one or more genes in the gene set and none of the one or more genes in the gene set of the test cell comprises any mutation.

Another aspect of the invention is directed to methods of identifying a subject comprising cells that exhibit the phenocopy signature. The methods can comprise isolating a test cell from the subject, obtaining a gene expression profile for the test cell, and determining whether the gene expression profile for the test cell matches the phenocopy signature. The test cell exhibits the phenocopy signature if the gene expression profile for the test cell matches the phenocopy signature. The subject can be any organism, such as plants, animals, microbial colonies, etc. As used herein, “organism” encompasses any collection of interacting cells, including microbial colonies, microbiomes, etc. Exemplary animals include mammals, such as humans. The test cell can include any type of cell isolated from a subject. The test cell can be taken from any part of the subject's body or any tissue from the subject's body. Exemplary tissues include connective tissue, epithelial tissue, muscle tissue, and nervous tissue. Exemplary connective tissues include fat tissue. Exemplary epithelial tissues include the lining of the gastrointestinal tract and other hollow organs and skin. Exemplary muscle tissues include cardiac muscle tissue, smooth muscle tissue, and skeletal muscle tissue. Exemplary nervous tissues include brain tissue, spinal cord tissue, and nerve tissue. Exemplary test cells include stem cells, bone cells (e.g., osteoclasts, osteoblasts, osteocytes), blood cells (e.g., red blood cells, white blood cells, platelets), muscle cells (e.g., skeletal muscle cells, cardiac muscle cells, smooth muscle cells), fat cells, skin cells, nerve cells, endothelial cells, sex cells (e.g., sperm, ova), pancreatic cells (e.g., beta cells, alpha cells), and cancer cells, among others. In some versions, the test cell comprises a healthy cell. In some versions, the test cell comprises a pathological cell.

As above, in some versions, the test cell comprises each of the one or more genes in the gene set, and none of the one or more genes in the gene set of the test cell comprises any of the one or more mutations in the mutation set used to generate the phenocopy signature. In some versions, the test cell comprises each of the one or more genes in the gene set, and none of the one or more genes in the gene set of the test cell comprises any mutation that is pathogenic or predicted to be pathogenic. In some versions, the test cell comprises each of the one or more genes in the gene set, and none of the one or more genes in the gene set of the test cell comprises any coding mutation. In some versions, the test cell comprises each of the one or more genes in the gene set, and none of the one or more genes in the gene set of the test cell comprises any mutation.

Another aspect of the invention is directed to methods of predicting a subject sensitive to treatment with a drug that targets a biological pathway. In some versions, the methods comprise obtaining a gene expression profile for a test cell isolated from the subject and determining whether the gene expression profile for the test cell matches a phenocopy signature generated with a gene set from a biological pathway that the drug targets. The subject can be predicted to be sensitive to treatment with the drug if the gene expression profile for the test cell matches the phenocopy signature. The biological pathway can comprise any biological pathway, including any biological pathway described herein.

A drug is considered herein to target a particular biological pathway if treatment of a cell (which comprises treating a tissue, organ, or organism comprising the cell) with the drug affects (e.g., inhibits, enhances, etc.) the operation of the biological pathway. A drug targeting a particular biological pathway can do so directly or indirectly. Drugs that target the particular biological pathway directly interact (e.g., bind) at least one entity in the biological pathway. Drugs that target a particular biological pathway indirectly do not directly interact (e.g., bind) any entities in the biological pathway, and instead produces an effect in the cell that affects the biological pathway, in some cases nonspecifically (e.g, chemotherapy). Accordingly, in some versions, a drug targeting a particular biological pathway binds to at least one entity in the biological pathway. In some versions, a drug targeting a particular biological pathway binds to a gene or protein in the biological pathway. In some versions, a drug targeting a particular biological pathway does not bind to any entities in the biological pathway. Methods for identifying drugs that target particular biological pathways are known in the art. See, e.g., Rizi et al. 2021 (Fuzi B, Gurinova J, Hermjakob H, Ecker GF, Sheriff R. Path4Drug: Data Science Workflow for Identification of Tissue-Specific Biological Pathways Modulated by Toxic Drugs. Front Pharmacol. 2021 Oct. 14; 12:708296) and Pham et al. 2020 (Pham M, Wilson S, Govindarajan H, Lin CH, Lichtarge O. Discovery of disease- and drug-specific pathways through community structures of a literature network. Bioinformatics. 2020 Mar. 1; 36(6):1881-1888).

In some versions of the invention, the employed phenocopy signature is not determined by incorporating empirical data for the response of any training cells to the drug. As outlined in the following examples, the invention is capable of predicting response to drugs that target the biological pathway used to generate the phenocopy signature even if the phenocopy signature is generated without response data for drugs targeting the pathway.

As in the embodiments outlined, above, in some versions, the test cell comprises each of the one or more genes in the gene set, and none of the one or more genes in the gene set of the test cell comprises any of the one or more mutations in the mutation set. In some versions, the test cell comprises each of the one or more genes in the gene set, and none of the one or more genes in the gene set of the test cell comprises any mutation that is pathogenic or predicted to be pathogenic. In some versions, the test cell comprises each of the one or more genes in the gene set, and none of the one or more genes in the gene set of the test cell comprises any coding mutation. In some versions, the test cell comprises each of the one or more genes in the gene set, and none of the one or more genes in the gene set of the test cell comprises any mutation.

As in the embodiments outlined above, obtaining the gene expression profiles can comprise measuring the mRNA levels of different mRNA species present in the test cell and/or, depending on the availability of data for a particular test cell, downloading the gene expression profiles from a database, among other methods. In some versions, obtaining the gene expression profiles can comprise measuring mRNA levels of one or more of the genes in the biological pathway. In some versions, obtaining the gene expression profiles can comprise measuring mRNA levels of each of the genes in the biological pathway. In some versions, obtaining the gene expression profiles exclude measuring mRNA levels of one or more genes not in the biological pathway.

The test cell can be obtained from any body part, fluid, tissue, or organ of the subject comprising cells. The test cell preferably comprises the gene set used to generate the phenocopy signature. The test cell preferably comprises the biological pathway used to generate the phenocopy signature. In some versions, the methods comprise isolating the test cell from the subject.

In some versions, the biological pathway is a disease pathway, and the subject has a disease of the disease pathway. In some versions, the subject has a disease, and the test cell is pathological cell of the disease. In some versions, the biological pathway is a disease pathway, the subject has a disease of the disease pathway, and the test cell is pathological cell of the disease.

Some versions of the invention further comprising administering the drug to the subject if the subject is predicted to be sensitive to treatment with the drug. If the subject has a disease, such as a disease of the disease pathway, the drug is preferably administered in an amount effective to treat the disease. “Treat” and “treating” as used herein refers to any degree of amelioration of a disease or symptom thereof, including partial or complete remission.

The methods of the invention are capable of predicting subjects sensitive to treatment with a drug that targets a biological pathway by determining whether the gene expression profile for the test cell matches a phenocopy signature generated with a gene set from the biological pathway that the drug targets, without testing for mutations in the gene set or any other gene in the biological pathway. However, in some versions, predicting subjects sensitive to treatment with the drug can optionally further comprise obtaining a nucleic acid sequence of one or more genes in the biological pathway in the test cell and determining if the one or more genes comprises a mutation. The one or more genes in the test cell can be one or more of the genes in the gene set used to generate the phenocopy signature, or any other gene in the biological pathway.

Another aspect of the invention is directed to methods of predicting cells sensitive to treatment with drugs that target a biological pathway and, optionally, treating the cell. The methods can comprise obtaining a gene expression profile for a test cell, and determining whether the gene expression profile for the test cell matches a phenocopy signature generated with a gene set from the biological pathway that the drug targets. The cell is predicted to be sensitive to treatment with the drug if the gene expression profile for the test cell matches the phenocopy signature. The methods can further comprise administering the drug to the cell if the subject is predicted to be sensitive to treatment with the drug. Aspects outlined above regarding any elements or steps included in the present embodiment can be incorporated in the present embodiment.

In some versions, the phenocopy signature is a phenocopy signature of an EGFR pathway. In some versions, the phenocopy signature is a phenocopy signature of an EGFR mutation. In some versions, the phenocopy signature comprises a set of gene expression signatures from any 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or each of genes selected from the group consisting of HSP9OAA1, CDC37, AREG, GAB1, CBL, HBEGF, SOS1, PIK3CA, PLCG1, EREG, KRAS, EGF, RPS27A, PIK3R1, EGFR, UBC, SHC1, TGFA, UBB, HRAS, BTC, GRB2, EPGN, NRAS, and UBA52. In some versions, the phenocopy signature excludes a gene expression signature from a gene that is not one of HSP9OAA1, CDC37, AREG, GAB1, CBL, HBEGF, SOS1, PIK3CA, PLCG1, EREG, KRAS, EGF, RPS27A, PIK3R1, EGFR, UBC, SHC1, TGFA, UBB, HRAS, BTC, GRB2, EPGN, NRAS, and UBA52.

In some versions, the phenocopy signature is a phenocopy signature of a BRAF pathway. In some versions, the phenocopy signature is a phenocopy signature of a BRAF mutation. In some versions, the phenocopy signature comprises a set of gene expression signatures from any 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, or each of genes selected from the group consisting of BRAF, RAP1B, KSR1, ARRB1, HRAS, MARK3, ARRB2, CSK, MAP2K1, RAF1, MAPK1, NRAS, VWF, ARAF, MAPK3, RAP1A, VCL, IQGAP1, YWHAB, TLN1,

FN1, PEBP1, ACTG1, ACTB, CNKSR1, APBB1IP, SRC, ITGB3, ITGA2B, CNKSR2, KSR2, FGG, FGA, FGB, and KRAS. In some versions, the phenocopy signature excludes a gene expression signature from a gene that is not one of BRAF, RAP1B, KSR1, ARRB1, HRAS, MARK3, ARRB2, CSK, MAP2K1, RAF1, MAPK1, NRAS, VWF, ARAF, MAPK3, RAP1A, VCL, IQGAP1, YWHAB, TLN1, FN1, PEBP1, ACTG1, ACTB, CNKSR1, APBB1IP, SRC, ITGB3, ITGA2B, CNKSR2, KSR2, FGG, FGA, FGB, and KRAS.

In some versions, the phenocopy signature is a phenocopy signature of a PIK3-AKT pathway. In some versions, the phenocopy signature is a phenocopy signature of a mutation in any one, any two, or each of PIK3CA, AKT1, and AKT2. In some versions, the phenocopy signature comprises a set of gene expression signatures from any 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more 39 or more, 40 or more, or each of genes selected from the group consisting of GAB2, PIK3CB, FGFR2, FGFR3, FGF10, FGF22, FGF4, FGFR1, PIK3C3, FGF20, FLT3LG, TRIB3, FGF9, AKT2, FGF8, GAB1, FGF6, FGF1, FGF23, PIK3CA, FLT3, KL, KLB, FGFS, FGF2, FGF7, PDPK1, PIK3R1, PDE3B, FGF18, FGF17, THEM4, FGFR4, FGF19, FRS2, IRS1, GRB2, PTPN11, IRS2, PIK3R4, and FGF16. In some versions, the phenocopy signature excludes a gene expression signature from a gene that is not one of GAB2, PIK3CB, FGFR2, FGFR3, FGF10, FGF22, FGF4, FGFR1, PIK3C3, FGF20, FLT3LG, TRIB3, FGF9, AKT2, FGF8, GAB1, FGF6, FGF1, FGF23, PIK3CA, FLT3, KL, KLB, FGFS, FGF2, FGF7, PDPK1, PIK3R1, PDE3B, FGF18, FGF17, THEM4, FGFR4, FGF19, FRS2, IRS1, GRB2, PTPN11, IRS2, PIK3R4, and FGF16.

In some versions, the phenocopy signature is a phenocopy signature of a PARP/HRD pathway. In some versions, the phenocopy signature is a phenocopy signature of a mutation in any one or more, any two or more, any three or more, or each of BRCA1, BRCA2, PARP1, and PARP2. In some versions, the phenocopy signature comprises a set of gene expression signatures from any 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, 63 or more, 64 or more, 65 or more, 66 or more, 67 or more, 68 or more, 69 or more, 70 or more, 71 or more, 72 or more, 73 or more, 74 or more, 75 or more, 76 or more, 77 or more, 78 or more, 79 or more, 80 or more, 81 or more, 82 or more, 83 or more, 84 or more, 85 or more, 86 or more, 89 or more, 88 or more, 89 or more, 90 or more, 91 or more, or each of genes selected from the group consisting of RAD52, BRCA1, ERCC1, RFC1, RFC2, RAD51, POLD1, RNF4, TP53BP1, TIPIN, SIRT6, POLD3, PALB2, UIMC1, CLSPN, ABL1, POLE2, RBBP8, UBE2I, NBN, PIAS4, BABAM1, RPA3, POLD2, RAD51C, RAD51AP1, RFCS, TIMELESS, RNF8, RAD1, RAD50, POLE4, SUMO1, RPA2, POLK, CDK2, XRCC3, HERC2, RPA1, PCNA, CCNA1, RFC3, HUS1, BRIP1, MDC1, DNA2, BARD1, BRCA2, RPS27A, CCNA2, POLE3, ATM, CHEK1, PPP4C, UBC, RAD9B, RAD17, EME1, PPP4R2, TOPBP1, RFC4, RNF168, ATRIP, SPIDR, WRN, UBE2V2, UBB, POLH, RHN01, RAD9A, MUS81, KATS, EXO1, ATR, POLD4, ERCC4, RMI2, POLE, TOP3A, UBE2N, GEN1, RMI1, RAD51B, RAD51D, BRCC3, SUMO2, SLX4, XRCC2, BLM, EME2, UBA52, and RTEL1. In some versions, the phenocopy signature excludes a gene expression signature from a gene that is not one of RAD52, BRCA1, ERCC1, RFC1, RFC2, RAD51, POLD1, RNF4, TP53BP1, TIPIN, SIRT6, POLD3, PALB2, UIMC1, CLSPN, ABL1, POLE2, RBBP8, UBE2I, NBN, PIAS4, BABAM1, RPA3, POLD2, RAD51C, RAD51AP1, RFCS, TIMELESS, RNF8, RAD1, RAD50, POLE4, SUMO1, RPA2, POLK, CDK2, XRCC3, HERC2, RPA1, PCNA, CCNA1, RFC3, HUS1, BRIP1, MDC1, DNA2, BARD1, BRCA2, RPS27A, CCNA2, POLE3, ATM, CHEK1, PPP4C, UBC, RAD9B, RAD17, EME1, PPP4R2, TOPBP1, RFC4, RNF168, ATRIP, SPIDR, WRN, UBE2V2, UBB, POLH, RHNO1, RAD9A, MUS81, KATS, EXO1, ATR, POLD4, ERCC4, RMI2, POLE, TOP3A, UBE2N, GEN1, RMI1, RAD51B, RAD51D, BRCC3, SUMO2, SLX4, XRCC2, BLM, EME2, UBA52, and RTEL1.

In some versions, the phenocopy signature is a phenocopy signature of a MAPK pathway. In some versions, the phenocopy signature is a phenocopy signature of a mutation in any 1 or more, 2 or more, 3 or more, 4 or more, 5 or more, 6 or more, 7 or more, 8 or more, 9 or more, 10 or more, 11 or more, 12 or more, or each of MAPK11, MAPK12, MAPK13, MAPK14, MAPK3, MAPK1, MKNK1, MKNK2, MAP2K1, MAP2K2, MAPK8, MAPK9, and MAPK10. In some versions, the phenocopy signature comprises a set of gene expression signatures from any 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, 49 or more, 50 or more, 51 or more, 52 or more, 53 or more, 54 or more, 55 or more, 56 or more, 57 or more, 58 or more, 59 or more, 60 or more, 61 or more, 62 or more, or each of genes selected from the group consisting of MAP2K3, MAPK9, CUL1, TAB2, MAP2K4, MEF2A, RPS6KA2, FBXW11, MAP2K7, MEF2C, MAPK1, TAB1, RPS6KA5, MAPK3, RIPK2, IKBKB, PPP2CB, VRK3, PPP2R1A, NOD1, MAPK8, MAP3K8, DUSP3, MAP2K6, NFKB1, MAPK10, MAPK14, PPP2R5D, SKP1, PPP2CA, MAPKAPK3, ATF2, RPS6KA1, CREB1, DUSP4, ATF1, ELK1, IRAK2, MAP3K7, PPP2R1B, DUSP6, RPS27A, UBC, TAB3, MAPKAPK2, DUSP7, BTRC, MAPK7, NOD2, TNIP2, MAP2K1, UBB, FOS, TRAF6, RPS6KA3, JUN, UBE2N, IRAK1, MAPK11, CHUK, UBA52, UBE2V1, and IKBKG. In some versions, the phenocopy signature excludes a gene expression signature from a gene that is not one of MAP2K3, MAPK9, CUL1, TAB2, MAP2K4, MEF2A, RPS6KA2, FBXW11, MAP2K7, MEF2C, MAPK1, TAB1, RPS6KA5, MAPK3, RIPK2, IKBKB, PPP2CB, VRK3, PPP2R1A, NOD1, MAPK8, MAP3K8, DUSP3, MAP2K6, NFKB1, MAPK10, MAPK14, PPP2R5D, SKP1, PPP2CA, MAPKAPK3, ATF2, RPS6KA1, CREB1, DUSP4, ATF1, ELK1, IRAK2, MAP3K7, PPP2R1B, DUSP6, RPS27A, UBC, TAB3, MAPKAPK2, DUSP7, BTRC, MAPK7, NOD2, TNIP2, MAP2K1, UBB, FOS, TRAF6, RPS6KA3, JUN, UBE2N, IRAK1, MAPK11, CHUK, UBA52, UBE2V1, and IKBKG.

In some versions, the phenocopy signature is a phenocopy signature of a ERBB2 pathway. In some versions, the phenocopy signature is a phenocopy signature of a mutation in ERBB2. In some versions, the phenocopy signature comprises a set of gene expression signatures from any 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, or each of genes selected from the group consisting of MATK, FYN, ERBB3, RHOA, PTPN18, HSP9OAA1, PTK6, STUB1, AKT2, CDC37, GAB1, HBEGF, SOS1, AKT3, PIK3CA, PLCG1, EREG, PTPN12, DIAPH1, KRAS, USP8, EGF, ERBB2, GRB7, AKT1, RPS27A, PIK3R1, EGFR, UBC, PRKCA, NRG1, NRG2, SHC1, MEM01, PRKCD, CUL5, NRG4, UBB, PRKCE, HRAS, BTC, YES1, GRB2, ERBB4, RNF41, NRG3, SRC, NRAS, and UBA52. In some versions, the phenocopy signature excludes a gene expression signature from a gene that is not one of MATK, FYN, ERBB3, RHOA, PTPN18, HSP9OAA1, PTK6, STUB1, AKT2, CDC37, GAB1, HBEGF, SOS1, AKT3, PIK3CA, PLCG1, EREG, PTPN12, DIAPH1, KRAS, USP8, EGF, ERBB2, GRB7, AKT1, RPS27A, PIK3R1, EGFR, UBC, PRKCA, NRG1, NRG2, SHC1, MEMO1, PRKCD, CUL5, NRG4, UBB, PRKCE, HRAS, BTC, YES1, GRB2, ERBB4, RNF41, NRG3, SRC, NRAS, and UBA52.

In some versions, the phenocopy signature is a phenocopy signature of a MTOR pathway. In some versions, the phenocopy signature is a phenocopy signature of a mutation in MTOR. In some versions, the phenocopy signature comprises a set of gene expression signatures from any 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, or each of genes selected from the group consisting of RRAGD, EIF4B, STRADB, RRAGB, PPM1A, CAB39L, TSC2, EEF2K, AKT2, RHEB, PRKAG2, RPS6KB1, LAMTOR3, PRKAB1, EIF4G1, PRKAG3, LAMTOR2, RRAGC, STK11, PRKAB2, PRKAA1, LAMTOR5, CAB39, RPS6, RPTOR, AKT1, LAMTOR1, EIF4E, RRAGA, PRKAA2, TSC1, YWHAB, MLST8, SLC38A9, PRKAG1, EIF4EBP1, LAMTOR4, MTOR, AKT1S1, and STRADA. In some versions, the phenocopy signature excludes a gene expression signature from a gene that is not one of RRAGD, EIF4B, STRADB, RRAGB, PPM1A, CAB39L, TSC2, EEF2K, AKT2, RHEB, PRKAG2, RPS6KB1, LAMTOR3, PRKAB1, EIF4G1, PRKAG3, LAMTOR2, RRAGC, STK11, PRKAB2, PRKAA1, LAMTOR5, CAB39, RPS6, RPTOR, AKT1, LAMTOR1, EIF4E, RRAGA, PRKAA2, TSC1, YWHAB, MLST8, SLC38A9, PRKAG1, EIF4EBP1, LAMTOR4, MTOR, AKT1S1, and STRADA.

In some versions, the phenocopy signature is a phenocopy signature of a JAK pathway. In some versions, the phenocopy signature is a phenocopy signature of a mutation in any 1 or more, 2 or more, or each of JAK1, JAK2, and JAK3. In some versions, the phenocopy signature comprises a set of gene expression signatures from any 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, or each of genes selected from the group consisting of GAB2, PIK3CB, IL5RA, JAK2, CSF2RB, IL2RB, SOS2, IL21R, JAK3, IL2, PTPN6, ILS, STAT1, SOS1, PIK3R3, PTK2B, PIK3CA, IL9R, STATSA, IL2RA, IL15RA, HAVCR2, STAT4, IL21, PIK3R1, IL9, IL2RG, SHC1, JAK1, IL15, IL3, CSF2, SYK, INPPL1, STAT3, INPP5D, LGALS9, PIK3CD, STATSB, GRB2, LCK, IL3RA, and CSF2RA. In some versions, the phenocopy signature excludes a gene expression signature from a gene that is not one of GAB2, PIK3CB, IL5RA, JAK2, CSF2RB, IL2RB, SOS2, IL21R, JAK3, IL2, PTPN6, ILS, STAT1, SOS1, PIK3R3, PTK2B, PIK3CA, IL9R, STATSA, IL2RA, IL15RA, HAVCR2, STAT4, IL21, PIK3R1, IL9, IL2RG, SHC1, JAK1, IL15, IL3, CSF2, SYK, INPPL1, STAT3, INPP5D, LGALS9, PIK3CD, STATSB, GRB2, LCK, IL3RA, and CSF2RA.

In some versions, the phenocopy signature is a phenocopy signature of a TP53 pathway. In some versions, the phenocopy signature is a phenocopy signature of a mutation in any one or both of TP53 and MDM2. The mutation in TP53 can be a coding mutation in each TP53 gene or a coding mutation in one TP53 gene and a copy number (CN) loss. In some versions, the phenocopy signature comprises a set of gene expression signatures from any 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, 39 or more, 40 or more, 41 or more, 42 or more, 43 or more, 44 or more, 45 or more, 46 or more, 47 or more, 48 or more, or each of genes selected from the group consisting of CNOT4, RBL1, BAX, AURKA, CNOT3, PCBP4, EP300, E2F1, RGCC, RBL2, CCNE1, CDKN1B, CNOT2, CNOT6, TFDP2, ARID3A, GADD45A, PLAGL1, CDK2, CDKN1A, CNOT1, PRMT1, E2F8, PCNA, CCNA1, CCNB1, CNOT6L, TP53, CARM1, CCNA2, PLK2, TNKS1BP1, CENPJ, CNOT8, CDC25C,

CNOT11, BTG2, ZNF385A, E2F7, CDK1, PLK3, CCNE2, SFN, NPM1, CNOT10, TFDP1, CNOT7, and E2F4. In some versions, the phenocopy signature excludes a gene expression signature from a gene that is not one of CNOT4, RBL1, BAX, AURKA, CNOT3, PCBP4, EP300, E2F1, RGCC, RBL2, CCNE1, CDKN1B, CNOT2, CNOT6, TFDP2, ARID3A, GADD45A, PLAGL1, CDK2, CDKN1A, CNOT1, PRMT1, E2F8, PCNA, CCNA1, C CNB 1, CNOT6L, TP53, CARM1, CCNA2, PLK2, TNKS1BP1, CENPJ, CNOT8, CDC25C, CNOT11, BTG2, ZNF385A, E2F7, CDK1, PLK3, CCNE2, SFN, NPM1, CNOT10, TFDP1, CNOT7, and E2F4.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Example 1

### Identification of Phenocopies Improves Prediction of Targeted Therapy Response Over DNA Mutations Alone

### Summary

DNA mutations in specific genes can confer preferential benefit from drugs targeting those genes. However, other molecular perturbations can “phenocopy” pathogenic mutations, but would not be identified using standard clinical sequencing, leading to missed opportunities for other patients to benefit from targeted treatments. We hypothesized that RNA phenocopy signatures of key cancer driver gene mutations could improve the ability to predict response to targeted therapies, despite not being directly trained on drug response. To test this, we built gene expression signatures in tissue samples for specific mutations and found that phenocopy signatures broadly increased accuracy of drug response predictions in vitro compared to DNA mutation alone, and identified additional cancer cell lines that respond well with a positive/negative predictive value on par or better than DNA mutations. We further validated our results across four clinical cohorts. Our results show that routine RNA sequencing of tumors to identify phenocopies in addition to standard targeted DNA sequencing would improve the ability to accurately select patients for targeted therapies in the clinic.

### Introduction

Over the last decade, targeted therapies against a large range of oncogenic pathways have emerged as valuable additions to our anti-cancer armamentarium. These drugs tend to have a more favorable toxicity profile compared to cytotoxic chemotherapies^{1,2} and dozens of new therapies enter the market every year. Targeted therapies have demonstrated particular success in patients harboring specific driver mutations, usually in their respective targets^{3,4}.

The FDA has approved EGFR inhibitors in EGFR-mutant NSCLC⁵⁻⁷, BRAF inhibitors in both BRAF-mutant melanoma^{8,9} and NSCLC¹⁰, PI3K inhibitors in PIK3CA-mutant breast cancer¹¹, and PARP inhibitors in Homologous Recombination Deficient (HRD) ovarian¹² and prostate¹³ cancer.

For many of the genes with FDA approved biomarker indications, there are frequently known hotspot mutations, such as the V600 mutations in BRAF¹⁴. While the presence of these driver mutations tend to be informative for identifying patients for targeted therapies, there are often mutations of unknown significance which fall elsewhere in the gene that may or may not convey sensitivity¹⁵. Thus, the response in patients who harbor these mutations is not uniform, and many patients fail to respond even though they carry the driver mutation of interest¹⁶⁻²⁰. Additionally, others lacking a mutation may still show benefit from treatment. The reasons for the variability in response are multi-factorial. First, not all mutations alter the function of the protein and different mutations can have wildly different phenotypic impacts depending on the location and amino acid change. Second, regulation via epigenetic, post-transcriptional, and post-translational changes can modulate the impact of mutations and lead to incomplete penetrance of the expected phenotype. Finally, there may be other modes of activation for a particular oncogenic pathway upstream, downstream, or even in a different pathway independent of mutations in the target itself.

The activation of many oncogenic pathways leads to distinct transcriptomic changes. However, to date, work assessing gene expression patterns mimicking DNA alterations has been limited in scope to specific targets or cancer types. We hypothesized that gene expression signatures that identify phenocopies of alterations in key DNA alterations would improve predictions of response and resistance to targeted therapies. For example, these signatures could identify additional tumors which are a phenocopy of an EGFR-mutant tumor that would respond to anti-EGFR therapy, without necessarily carrying an EGFR mutation. Likewise, these phenocopy signatures could also identify tumors with an EGFR mutation of unknown significance that do not display the EGFR-mutant phenocopy, and do not respond to anti-EGFR therapy.

Herein, we develop phenocopy signatures of mutations in key cancer genes on 9248 patient samples across cancer types and validate in 1982 cell line experiments across three datasets that these signatures improve our ability to predict response to targeted therapies compared to DNA mutations alone. We also demonstrate that these phenocopy signatures predict response in clinical cohorts, and shift under the selective pressure of treatment. Unlike the previous literature in this area²¹⁻³³, we do not directly train models to predict drug response. Instead, the association of drug response to our phenocopy signatures arise as an indirect but intended side effect.

### Methods

We trained our phenocopy signatures on DNA alterations in the clinical TCGA dataset, which has minimal treatment response information. This is possible because we are not directly training on drug response, and our indirect approach has an added benefit in allowing us to save all cell line and clinical datasets with drug response for validation without having to worry about information leakage. Previous approaches directly training on cell line drug response face challenges in identifying suitable validation cohorts, as many of the cell lines overlap between different cell line datasets and clinical validation cohorts are rare.

### DNA Mutation Annotation

DNA mutations were annotated with Annovar⁴⁹. Only protein sequence-altering mutations were included. Silent, splicing, intronic, upstream, and downstream mutations were excluded from our analysis. To identify mutations with stronger evidence for being pathogenic, ClinVar and various computational tools (SIFT, Polyphen-2 HVAR, Polyphen-2 HDIV, and FATHMNI) were used. A sample was considered to have a pathogenic mutation if predicted by any of the computational tools or marked as pathogenic or likely pathogenic by ClinVar. A total of eight oncogenic signaling pathways with targeted drugs and mutations in the key driver genes were assessed (EGFR, BRAF, PI3K-AKT, PARP/HRD, ERBB2, mTOR, JAK, and MAPK). EGFR mutations were assessed for the EGFR pathway. BRAF mutations were assessed for the BRAF pathway. PIK3CA, AKT1, and AKT2 mutations were assessed for the PIK3-AKT pathway. BRCA1/2 and PARP1/2 mutations were assessed for the PARP/HRD pathway. ERBB2 mutations were assessed for the ERBB2 pathway. MTOR mutations were assessed for the MTOR pathway. JAK1/2/3 mutations were assessed for the JAK pathway. MAPK11, MAPK12, MAPK13, MAPK14, MAPK3, MAPK1, MKNK1, MKNK2, MAP2K1, MAP2K2, MAPK8, MAPK9, and MAPK10 were assessed for the MAPK pathway. While amplifications and deletions are also important, we chose not to include these for training due to the lack of consistent thresholds for determining when a copy number change influences function, as well as the significant effects of tumor purity on copy number in the clinical samples.

### Phenocopy Signature Training

Prior to training the phenocopy signature, we filtered each dataset to only include genes within the pathway of interest as determined by the Reactome⁵⁰ database of gene pathways (Table 1). For each gene pathway, we removed cancer types with an alteration rate below 5% from our TCGA training dataset. We then used a gradient tree boosting approach to train phenocopy signatures which predicted mutation status (true or false) based on RNA expression. Gradient tree boosting is an ensemble learning method where decision trees are constructed to minimize a differentiable loss function. This is done through a gradient descent algorithm where trees are iteratively fit to the direction of steepest descent of the loss function. We trained our signature on the TCGA dataset using the R XGboost package (version 1.4.0.1). XGboost offers a GPU-based implementation of gradient tree boosting that leverages a histogram algorithm to find candidate splits. We applied this approach with a hinge loss function and used 10-fold cross validation to tune the depth and number of trees, with model accuracy assessed using Receiver Operator Curve (ROC) Area Under the Curve (AUC). A total of eight phenocopy signatures were trained, one for each oncogenic signaling pathway, and were locked prior to independent validation.

TABLE 1
Genes within pathways of interest.

| Pathway | EGFR | BRAF | PI3K_AKT | PARP7HRD | MAPK | ERBB2 | MTOR | JAK |
|---|---|---|---|---|---|---|---|---|
| Genes | HSP90AA1 | BRAF | GAB2 | RAD52 | MAP2K3 | MATK | RRAGD | GAB2 |
| | CDC37 | RAP1B | PIK3OB | BRCA1 | MAPK9 | FYN | EIFAB | PIK3CB |
| | AREG | KSR1 | FGFR2 | ERCC1 | CUL1 | ERBB3 | STRADB | IL5RA |
| | GAB1 | ARRB1 | FGFR3 | RFC1 | TAB2 | RHOA | RRAGB | JAK2 |
| | OBL | HRAS | FGF10 | RFC2 | MAP2K4 | PIPN18 | PPM1A | CSF2RB |
| | HBEGF | MARK3 | FGF22 | RAD51 | MEF2A | HSP90AA1 | CAB39L | IL2RB |
| | SOS1 | ARRB2 | FGF4 | POLD1 | RPS6KA2 | PIK6 | TSC2 | SOS2 |
| | PIK3CA | CSK | FGRR1 | RNF4 | FBXW11 | STUB1 | EEF2K | IL21R |
| | PLCG1 | MAP2K1 | PIK3C3 | TP53BP1 | MAP2K7 | AKT2 | AKT2 | JAK3 |
| | EREG | RAF1 | FGF20 | TIPIN | MEF2C | CDC37 | RHEB | IL2 |
| | KRAS | MAPK1 | FLT3LG | SIRT6 | MAPK1 | GAB1 | PRKAG2 | PIPN6 |
| | EGF | NRAS | TRIB3 | POLD3 | TAB1 | HBEGF | RPS6KB1 | IL5 |
| | RPS27A | VWF | FGF9 | PALB2 | RPS6KA5 | SOS1 | LAMTOR3 | STAT1 |
| | PIK3R1 | ARAF | AKT2 | UIMC1 | MAPK3 | AKT3 | PRKAB1 | SOS1 |
| | EGER | MAPK3 | FGF8 | CLSPN | RIPK2 | PIK3CA | EIF4G1 | PIK3R3 |
| | UBC | RAP1A | GAB1 | ABL1 | IKBKB | PLCG1 | PRKAG3 | PTK2B |
| | SHC1 | VCL | FGF6 | POLE2 | PPP2CB | EREG | LAMTOR2 | PIK3CA |
| | TGFA | IQGAP1 | FGF1 | RBBP8 | VRK3 | PIPN12 | RRAGC | IL9R |
| | UBB | YWHAB | FGF23 | UBE2I | PPP2R1A | DIAPH1 | STK11 | STAT5A |
| | HRAS | TLN1 | PIK3CA | NBN | NOD1 | KRAS | PRKAB2 | IL2RA |
| | BTC | FN1 | FLT3 | PIAS4 | MAPK8 | USP8 | PRKAA1 | IL15RA |
| | GRB2 | PEBP1 | KL | BABAM1 | MAP3K8 | EGF | LAMTOR5 | HAVCR2 |
| | EPGN | ACTG1 | KLB | RPA3 | DUSP3 | ERBB2 | CAB39 | STAT4 |
| | NRAS | ACTB | FGF5 | POLD2 | MAP2K6 | GRB7 | RPS6 | IL21 |
| | UBA52 | CNKSR1 | FGF2 | RAD51C | NFKB1 | AKT1 | RPTOR | PIK3R1 |
| | | APBB1IP | FGF7 | RAD51AP1 | MAPK10 | RPS27A | AKT1 | IL9 |
| | | SRC | PDPK1 | RFC5 | MAPK14 | PIK3R1 | LAMTOR1 | IL2RG |
| | | ITGB3 | PIK3R1 | TIMELESS | PPP2R5D | EGFR | EIF4E | SHC1 |
| | | IIGA2B | PDE3B | RNF8 | SKP1 | UBC | RRAGA | JAK1 |
| | | CNKSR2 | FGF18 | RAD1 | PPP2CA | PRKCA | PRKAA2 | IL15 |
| | | KSR2 | FGF17 | RAD50 | MAPKAPK3 | NRG1 | TSC1 | IL3 |
| | | FGG | THEM4 | POLE4 | ATF2 | NRG2 | YWHAB | CSF2 |
| | | FGA | FGFR4 | SUMO1 | RPS6KA1 | SHC1 | MLST8 | SYK |
| | | FGB | FGF19 | RPA2 | CREB1 | MEMO1 | SLC38A9 | INPPL1 |
| | | KRAS | FRS2 | POLK | DUSP4 | PRKOD | PRKAG1 | STAT3 |
| | | | IRS1 | CDK2 | ATF1 | CUL5 | EIF4EBP1 | INPP5D |
| | | | GRB2 | XRCC3 | ELK1 | NRG4 | LAMTOR4 | LGALS9 |
| | | | PIPN11 | HERC2 | IRAK2 | UBB | MTOR | PIK3OD |
| | | | IRS2 | RPA1 | MAP3K7 | PRKCE | AKT1S1 | STAT5B |
| | | | PIK3R4 | PCNA | PPP2R1B | HRAS | STRADA | GRB2 |
| | | | FGF16 | CCNA1 | DUSP6 | BTC | | LOK |
| | | | | RFC3 | RPS27A | YES1 | | IL3RA |
| | | | | HUS1 | UBC | GRB2 | | CSF2RA |
| | | | | BRIP1 | TAB3 | ERBB4 | | |
| | | | | MDC1 | MAPKAPK2 | RNF41 | | |
| | | | | DNA2 | DUSP7 | NRG3 | | |
| | | | | BARD1 | BTRC | SRC | | |
| | | | | BRCA2 | MAPK7 | NRAS | | |
| | | | | RPS27A | NOD2 | UBA52 | | |
| | | | | CONA2 | TNIP2 | | | |
| | | | | POLE3 | MAP2K1 | | | |
| | | | | ATM | UBB | | | |
| | | | | CHEK1 | FOS | | | |
| | | | | PPP4C | TRAF6 | | | |
| | | | | UBC | RPS6KA3 | | | |
| | | | | RAD9B | JUN | | | |
| | | | | RAD17 | UBE2N | | | |
| | | | | EME1 | IRAK1 | | | |
| | | | | PPP4R2 | MAPK11 | | | |
| | | | | TOPBP1 | CHUK | | | |
| | | | | RFC4 | UBA52 | | | |
| | | | | RNF168 | UBE2V1 | | | |
| | | | | ATRIP | IKBKG | | | |
| | | | | SPIDR | | | | |
| | | | | WRN | | | | |
| | | | | UBE2V2 | | | | |
| | | | | UBB | | | | |
| | | | | POLH | | | | |
| | | | | RHINO1 | | | | |
| | | | | RAD9A | | | | |
| | | | | MUS81 | | | | |
| | | | | KAT5 | | | | |
| | | | | EXO1 | | | | |
| | | | | ATR | | | | |
| | | | | POLD4 | | | | |
| | | | | ERCC4 | | | | |
| | | | | RMI2 | | | | |
| | | | | POLE | | | | |
| | | | | TOP3A | | | | |
| | | | | UBE2N | | | | |
| | | | | GEN1 | | | | |
| | | | | RMI1 | | | | |
| | | | | RAD51B | | | | |
| | | | | RAD51D | | | | |
| | | | | BRCC3 | | | | |
| | | | | SUMO2 | | | | |
| | | | | SLX4 | | | | |
| | | | | XRCC2 | | | | |
| | | | | BLM | | | | |
| | | | | EME2 | | | | |
| | | | | UBA52 | | | | |
| | | | | RTEL1 | | | | |

### Independent Validation of the Phenocopy Signatures in GDSC, CCLE, and DepMap

Each of the eight oncogenic pathways were tested in the GDSC, CCLE, and DepMap cohorts. Response for drugs specifically targeting each pathway was assessed per pathway as above. Mutations were assessed as above. The phenocopy signatures were applied without modification to the GDSC/CCLE/DepMap datasets and resulted in predicted mutation status to identify phenocopies. GDSC, CCLE, and DepMap sets were validated independently. As all three study cancer cell lines and anti-cancer drugs, there is overlap. However, as the experiments were done at different times with different techniques, we chose to investigate them as independent datasets.

### Statistical Approach

To compare whether the phenocopy signatures improved the ability to predict response to targeted therapies, we created linear models with the actual drug response (Z-score for the IC-50 for GDSC, the ActArea for CCLE, AUC for DepMap) as the dependent variable, and the actual and predicted mutation status as the independent variables. For the CCLE, IC-50 was not utilized due to 55% of all IC-50 values being the maximum tested concentration of 8 μM, therefore activity area (ActArea) was used, where a higher ActArea corresponds to increased sensitivity²³. For DepMap, 69% of IC-50 values were reported as NA, thus AUC was used as a measure of drug response. While using the same drug response metrics across datasets would have been ideal, diverse measures can provide complementary information even with the same cell lines/drugs and ensure our results are independent of the dataset. Model fit was determined using the ordinary least squares approach. Coefficients from the model indicate how strongly the actual and predicted alteration statuses contribute to drug response. We also performed a likelihood-ratio test using the chi-square statistic (χ²) to compare a single parameter model (mutation status alone) and a two parameter (mutation status and the phenocopy signature) model in order to assess if the phenocopy signature was significantly adding to DNA mutations alone in predicting drug response. Because the models are nested, the degrees of freedom equal the difference in the number of free parameters in the two models.

Thus, the two parameter model is a significant improvement over the single parameter model if the observed χ² statistic >4.5 corresponding to a Benjamini-Hochberg FDR multiple testing corrected p-value cutoff of 0.05.

Sensitivity and Specificity of the Phenocopy Signature in Predicting Drug Response

We next assessed the sensitivity and specificity of the phenocopy signatures. Because drug response was a continuous variable in our cell line datasets, we stratified “responders” and “non-responders” based on the top quartile vs. the bottom three quartiles⁵¹. To better understand the performance in the context of DNA mutations, we considered three subgroups: 1) cell lines without mutations, 2) cell lines with mutations that were not predicted to be pathogenic (e.g. unknown clinical significance) and 3) cell lines with mutations predicted to be pathogenic. We then compared this to the sensitivity and specificity of mutations alone, or pathogenic mutations alone.

### Data Availability

Processed DNA and RNA sequencing data from the Cancer Genome Atlas (TCGA) were downloaded using the UCSC Xena browser (xena.ucsc.edu). Processed DNA and RNA sequencing data and drug response data for the Genomic of Drug Sensitivity in Cancer (GDSC)⁴⁶ were downloaded from the GDSC web site (www.cancerrxgene.org). Processed DNA and RNA sequencing data and drug response data for the Cancer Cell Line Encyclopedia (CCLE)²³ were downloaded from the CCLE web site (portals.broadinstitute.org/ccle). The Cancer Dependency Map (DepMap) 2 shares the same cell lines and therefore DNA and RNA sequencing data as the CCLE, but independently tests treatment response, and these were obtained from the DepMap website (depmap.org). As recommended by DepMap, the MTS010 dataset was used for drug response data. Datasets were then filtered to only include the genes present in all three datasets. To allow comparability between groups, gene expression was normalized as previously described⁵². Gene expression was treated as a continuous variable throughout the examples. DNA mutation calls for TCGA, CCLE (including DepMap), and GDSC, were used as described in each dataset. Clinical datasets⁴²⁻⁴⁵ were downloaded from the Gene Expression Omnibus (www.ncbi.nlm.nih.gov/geo) with the following accession numbers: GSE50509, GSE65185, GSE99898, and GSE99898.

### Results

### Model Design

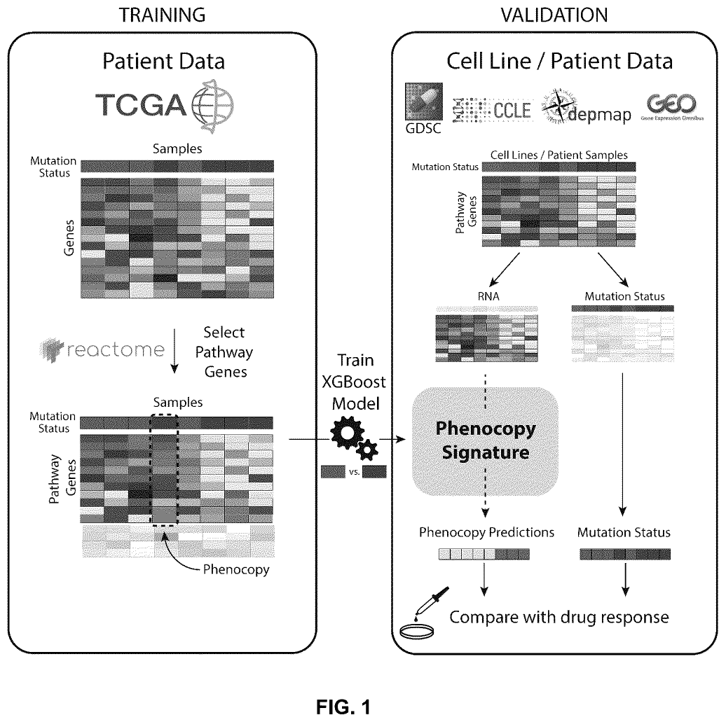

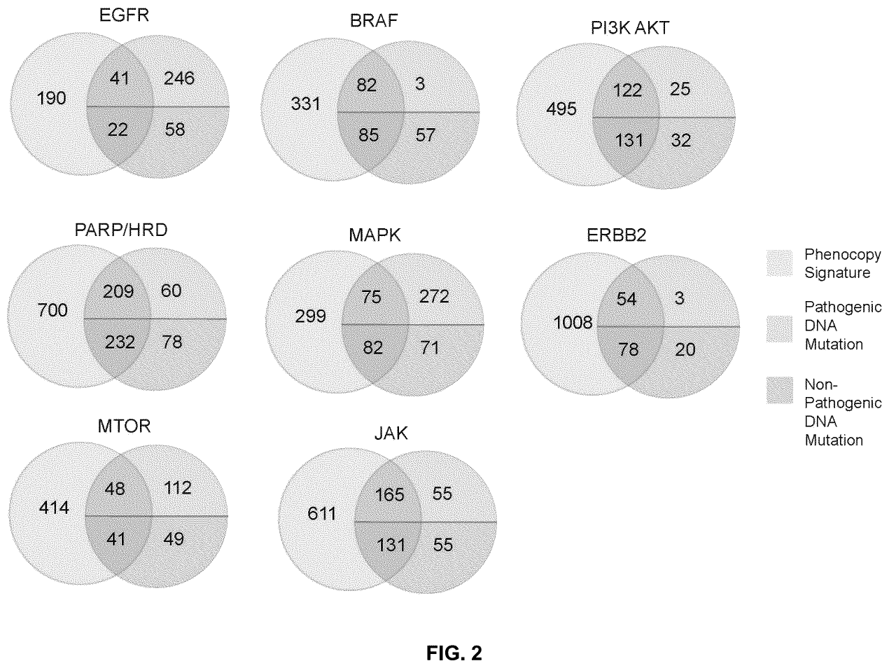

We first sought to define expression-based “phenocopy” signatures for various DNA mutations in therapeutically actionable pathways in cancer (FIG. 1). We designed the phenocopy signatures to identify RNA expression patterns from the “average” mutated tumor. To build these phenocopy signatures, we utilized publicly available data from the TCGA, which contains mutation status and RNA expression data for over 11000 tumor samples across 33 different tumor types. For each actionable gene, an XGBoost model was trained using gene expression profiles of pan-cancer tumor samples paired with the known DNA alteration status.

Each model was then trained to define a gene expression signature for eight different targetable pathways (EGFR, BRAF, PI3K-AKT, PARP/HRD, ERBB2, mTOR, JAK, and MAPK). To assess if the phenocopy signatures could predict drug response, independent data from the GDSC, CCLE and DepMap datasets were used which contain gene expression, DNA mutations, and drug responses across 969, 917, and 578 cancer cell lines, respectively.

Additionally, we analyzed four clinical studies which have gene expression and treatment data for patients treated with a drug targeting one of the pathways listed above.

### Phenocopy Signature Predictions

After assigning a predicted alteration status to each cell line in the testing set with the XGBoost-driven model as described above, we investigated how many cell lines in our validation cohorts were marked as altered by the phenocopy signature alone, the DNA mutation status alone, or by both. DNA alterations were additionally split into mutations which have a known or predicted deleterious or pathogenic effect and those with unknown significance (FIG. 2). Our goal was not to create signatures that would perfectly predict cell lines' alteration status, as this would not offer additional insights. Instead, we created our phenocopy signatures so they would identify cell lines that phenotypically mimicked gene expression patterns of altered cell lines, whether or not they carried a canonical driver mutation. For all the pathways, we found discordance between actual DNA mutation status and phenocopy predictions, which suggests that there is additional information from the phenocopy signatures that may help inform drug response predictions.

### Phenocopy Signatures Improve Pan-Cancer Drug Response Predictions Across Multiple Pathways

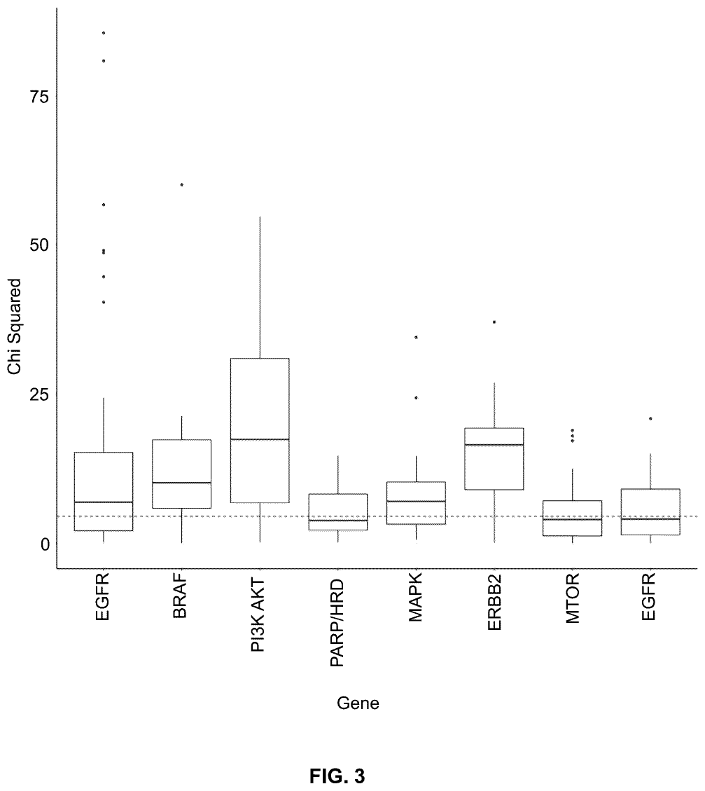

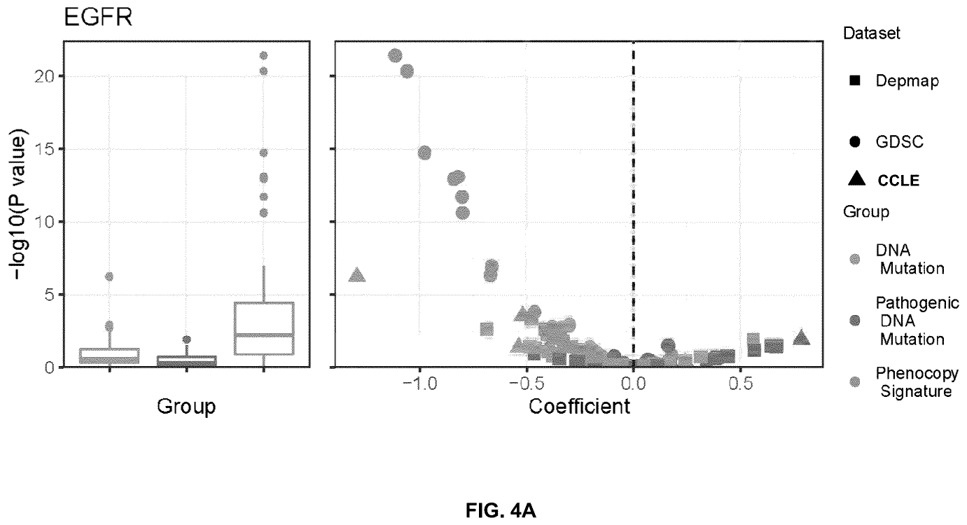

Next, we assessed how the gene-expression based phenocopy signatures performed in adding predictive information on targeted therapy drug response compared to DNA alterations alone. To assess if the discordance between actual DNA mutation status and the phenocopy signature predictions improves predictions of drug response, we chose to assess eight different pathways: four of which have clinically actionable mutations in various cancer types (BRAF⁸⁻¹⁰, BRCA^{13,3435}, EGFR⁵⁻⁷, and PIK3CA¹¹) and four of which are targets of ongoing research, but do not yet have FDA-approved indications (MAPK^{36,37}, ERBB^{38,39}, mTOR⁴⁰, and JAK⁴¹). We next tested if the phenocopy signatures improved the ability to predict drug response for drugs targeting these pathways. To accomplish this, we examined linear models of drug response to treatment targeting each pathway in the independent GDSC, CCLE, and DepMap cohorts, with both the true DNA alteration status and the phenocopy signatures as independent variables. To assess significance, a multiple-testing FDR-corrected chi-squared statistic was calculated for each drug/gene combination to determine if the addition of the phenocopy signature to DNA alterations alone improved the ability to predict drug response. Overall, model performance was significantly improved in 68% of cases across 165 different therapies targeting these eight pathways (FIG. 3). For 61% of drugs targeting EGFR, 75% of drugs targeting BRAF, 80% of drugs targeting PI3K-AKT, 50% of drugs targeting PARP/HRD, 64% of drugs targeting MAPK, 90% of drugs targeting ERBB2, 53% of drugs targeting mTOR, and 50% of drugs targeting JAK, the phenocopy signatures significantly added to DNA mutations alone.

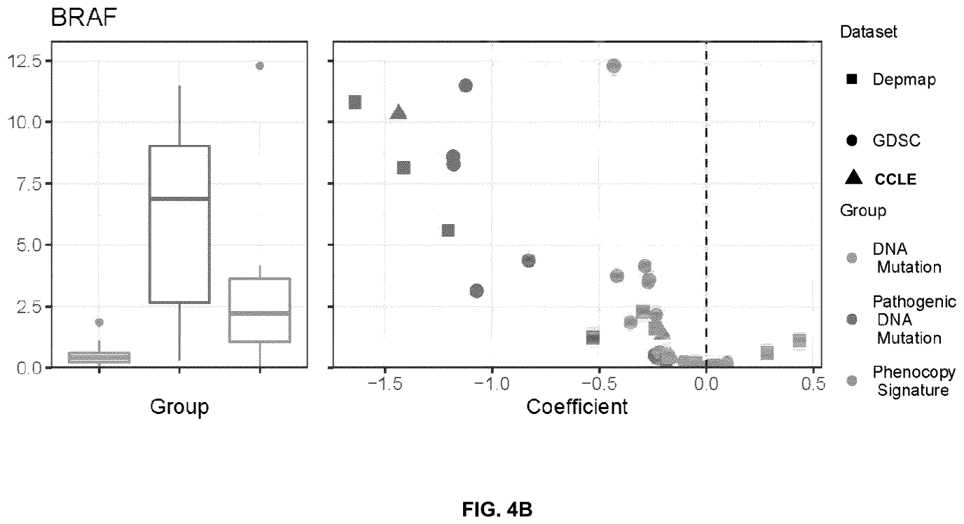

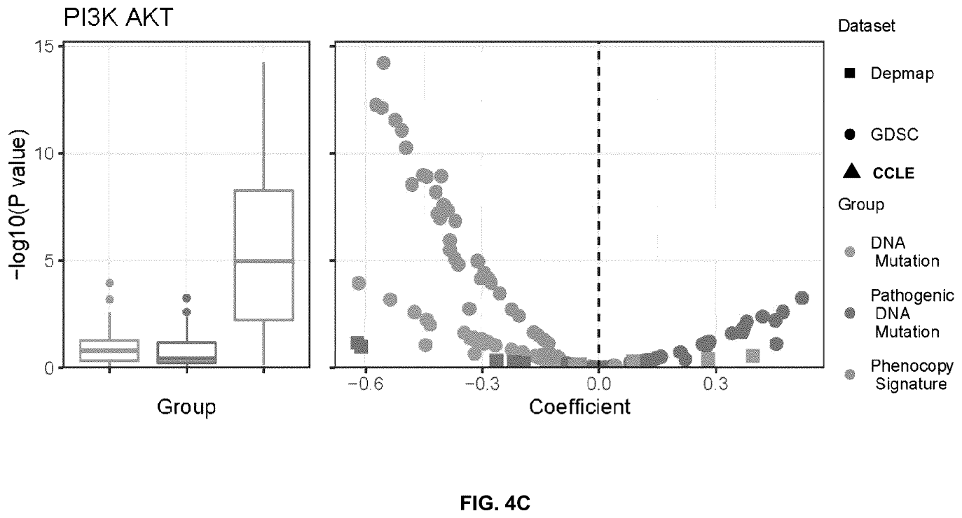

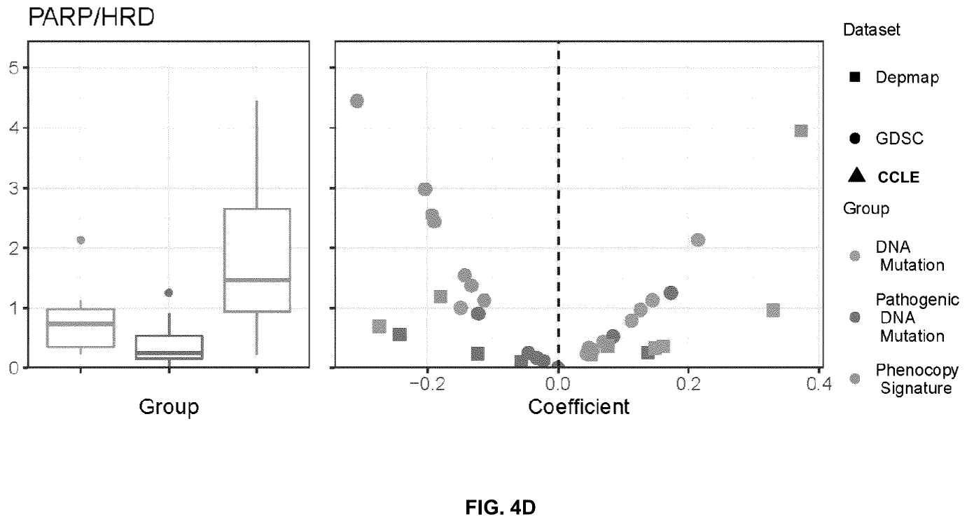

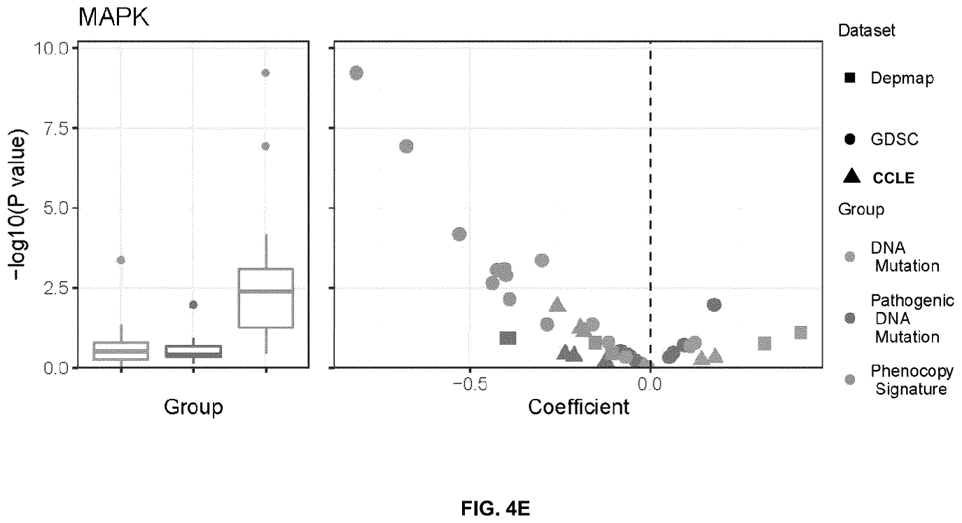

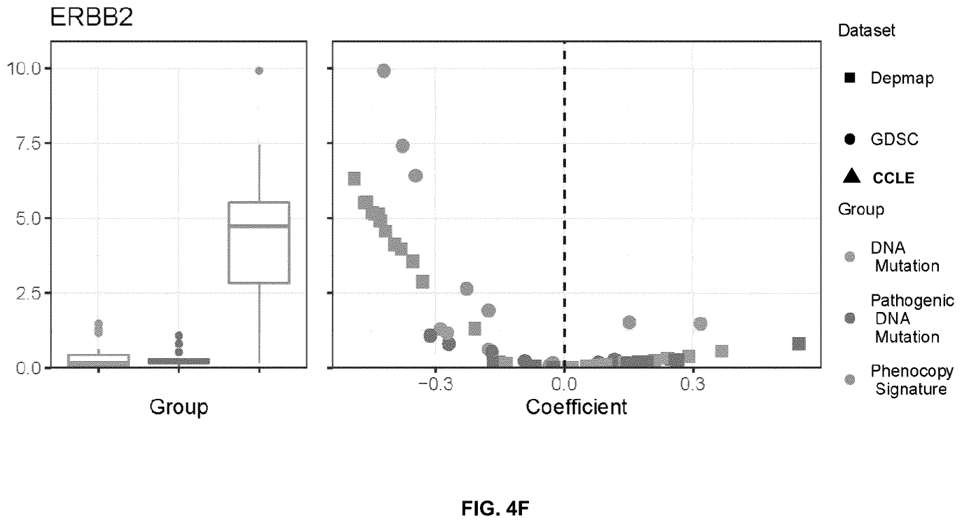

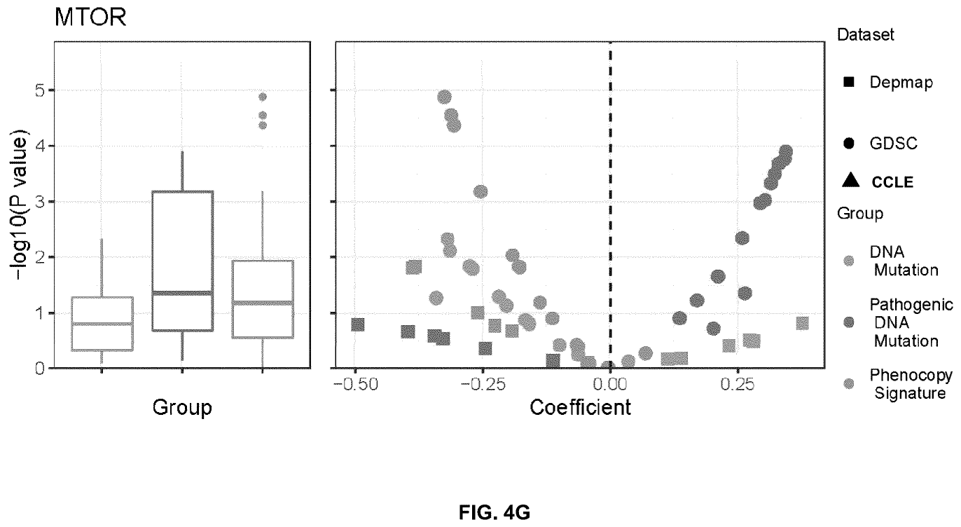

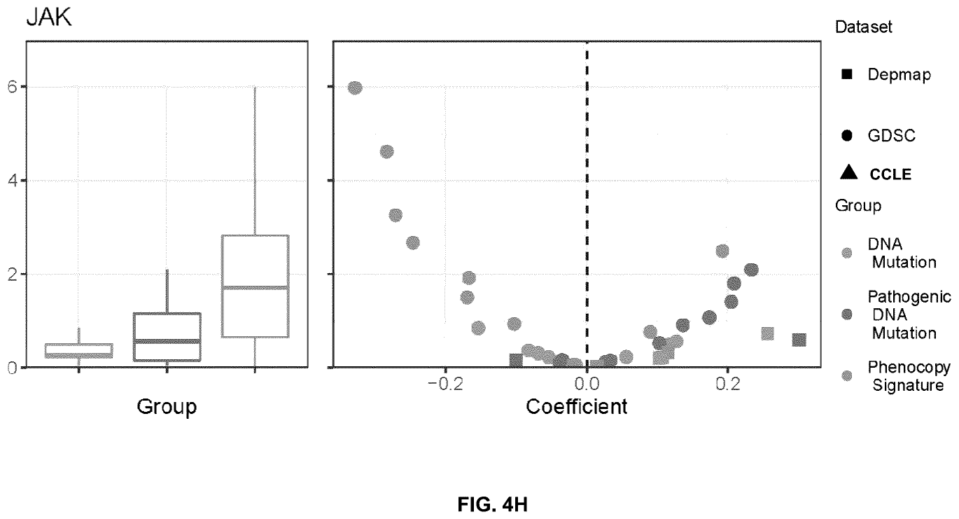

We next sought to further examine the individual pathways and drugs in more detail. Volcano plots of the contributions of the phenocopy signatures, DNA mutations, and pathogenic mutations in the linear models redemonstrated how the phenocopy signatures added to DNA mutations for drugs targeting pathways with and without mutations as FDA indications (FIGS. 4A-4H). Of note, negative coefficients represent expected estimates, where the actual mutation status or predicted mutation status from the phenocopy signature is associated with increased sensitivity to the drug. BRAF pathogenic mutations in particular successfully predicted response to BRAF inhibitors even after taking into account the phenocopy signatures, though the phenocopy signatures still demonstrated independent predictive signal. However, for the other pathways, phenocopy signatures generally out-performed DNA mutations (pathogenic or otherwise) in predicting response to targeted drugs across multiple agents and gene targets. These results are particularly impressive given that the phenocopy signatures were not directly trained to predict drug response, and instead appear to do so simply by virtue of their biological imperative, which is to identify phenocopies of DNA alterations.

### Sensitivity, Specificity, PPV, and NPV of Phenocopy Signatures

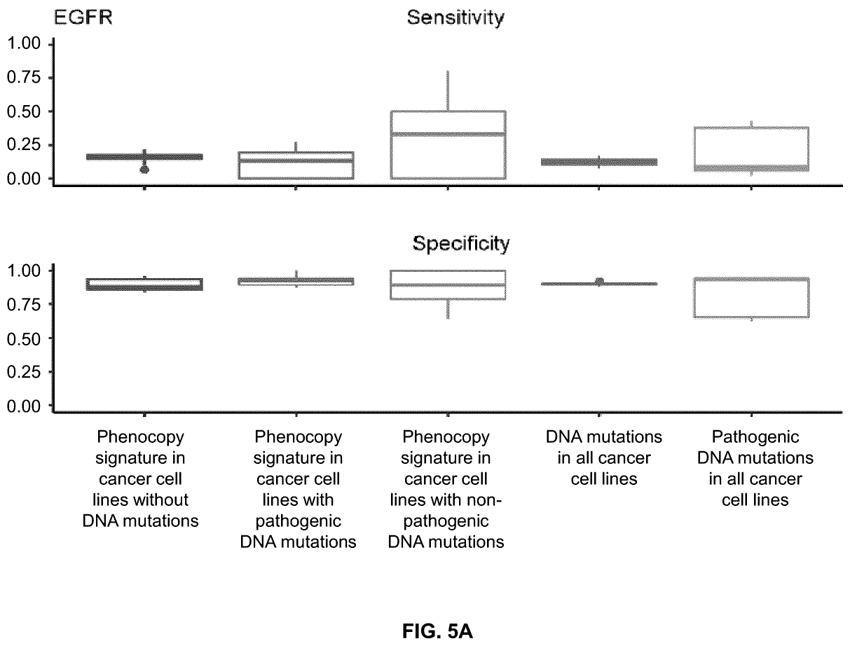

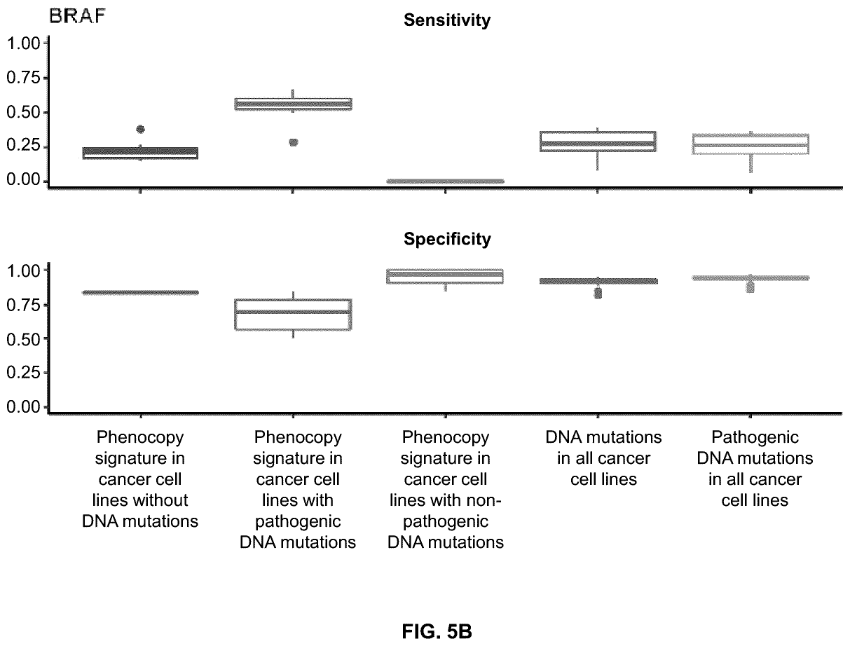

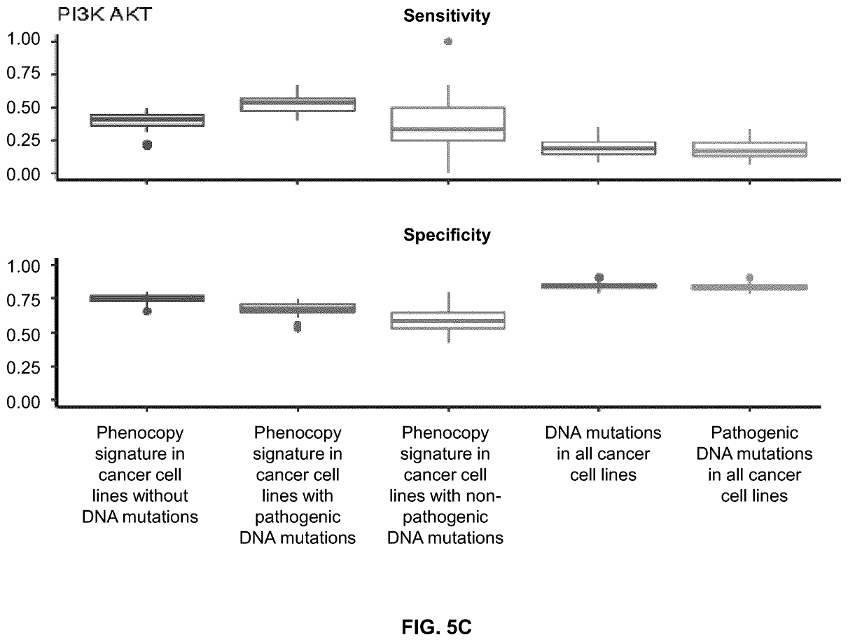

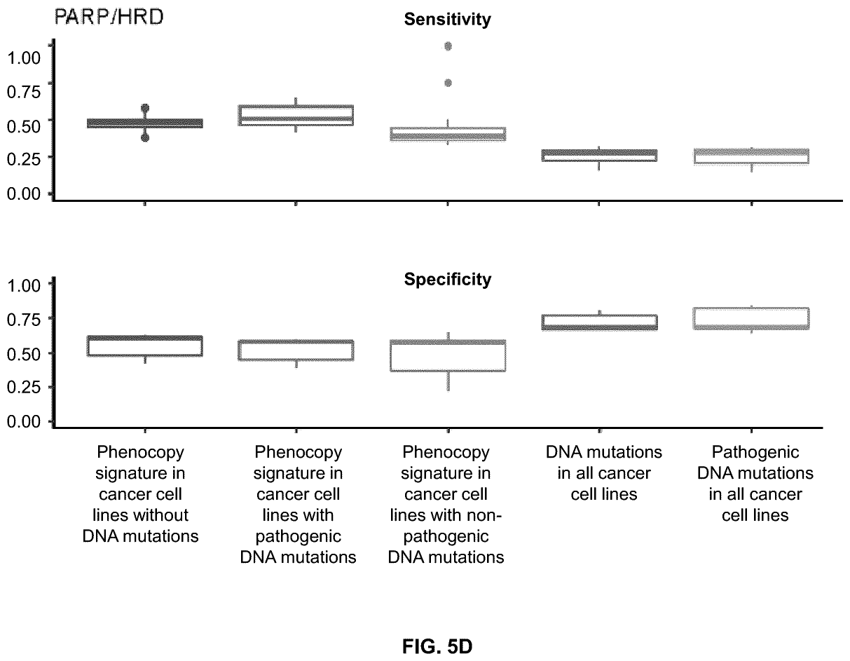

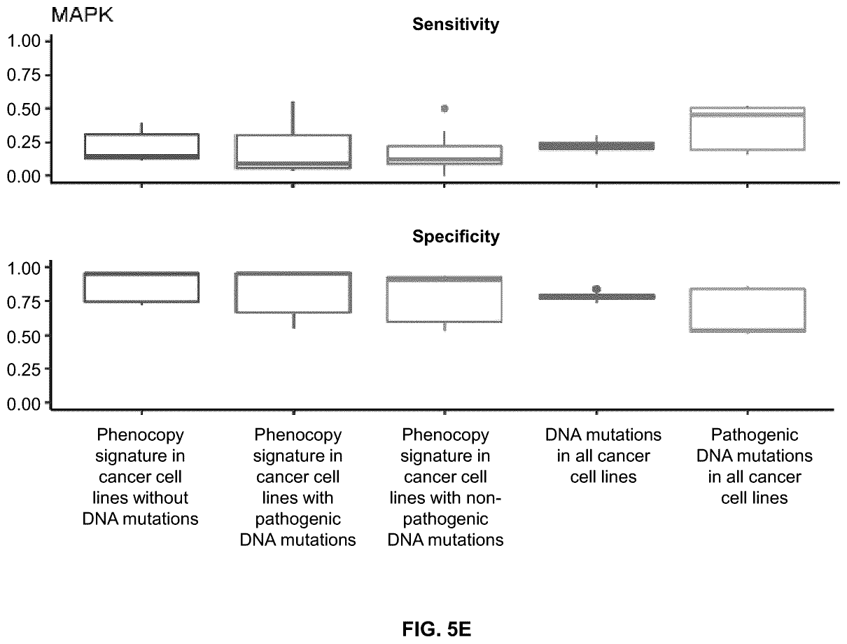

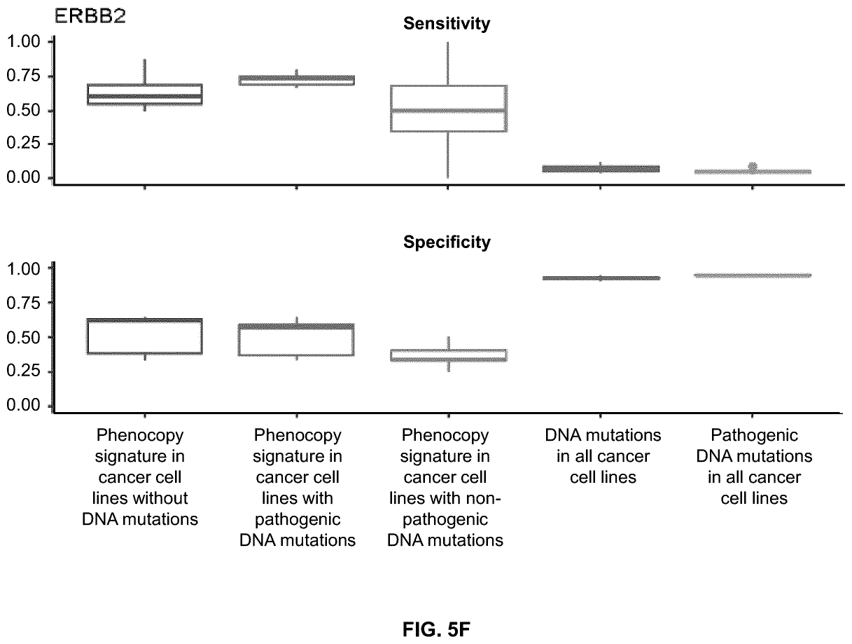

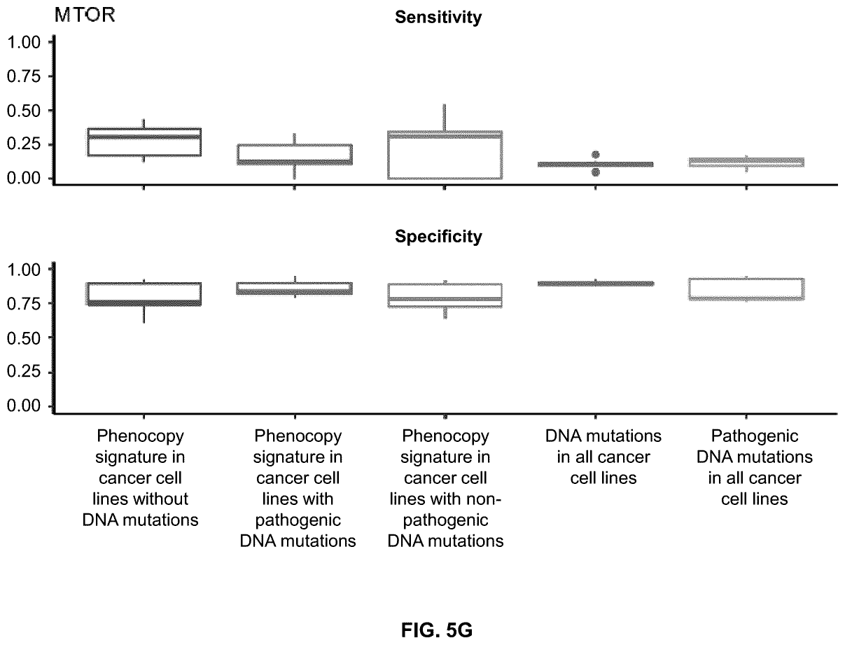

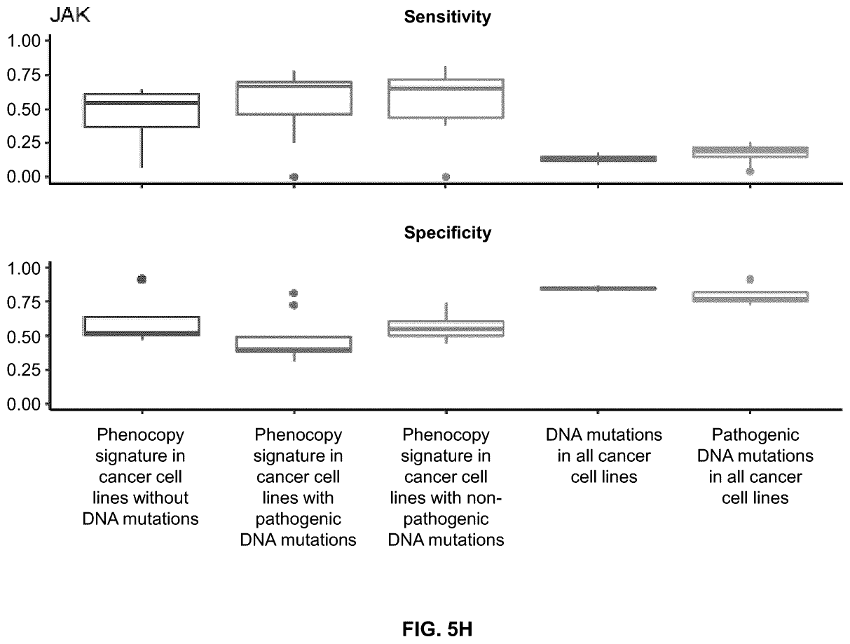

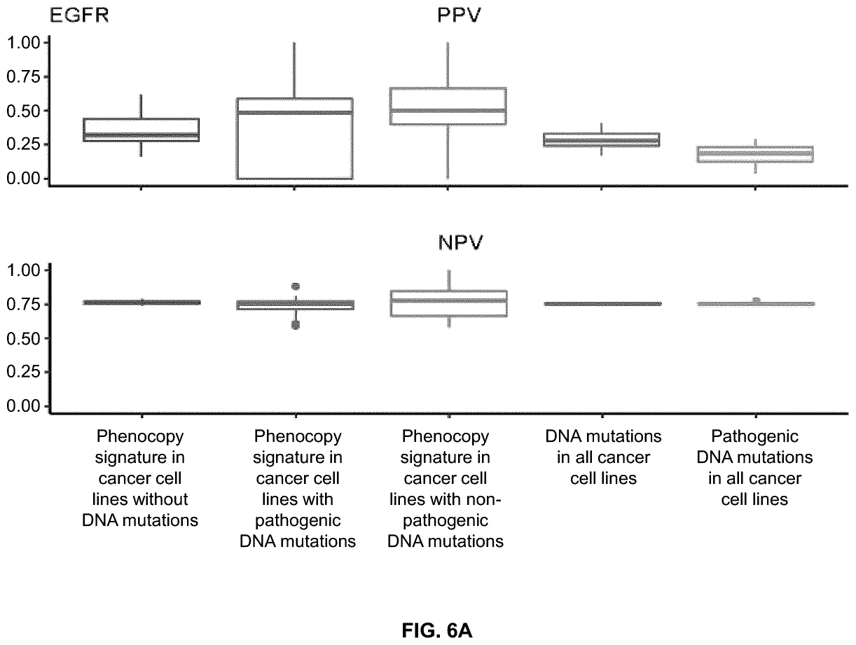

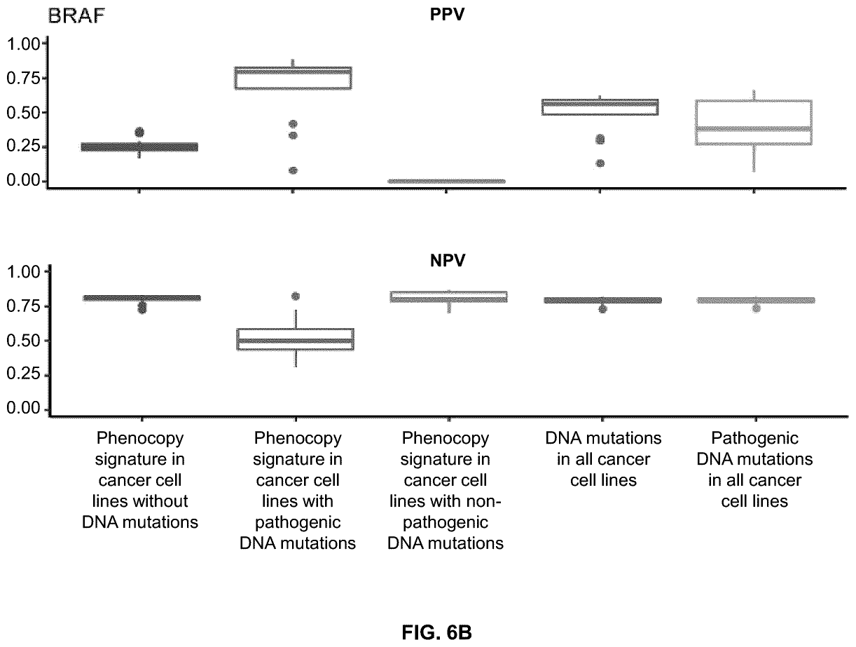

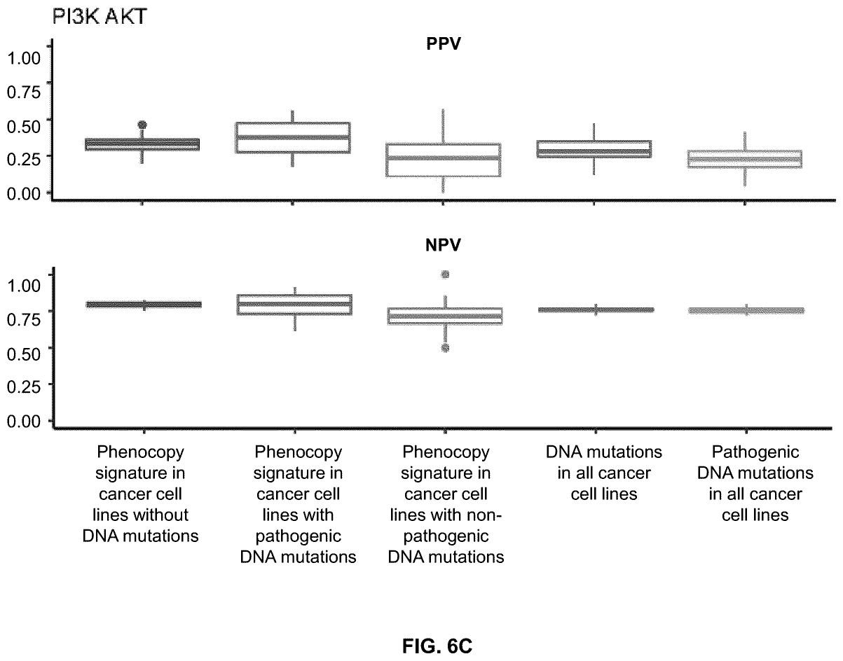

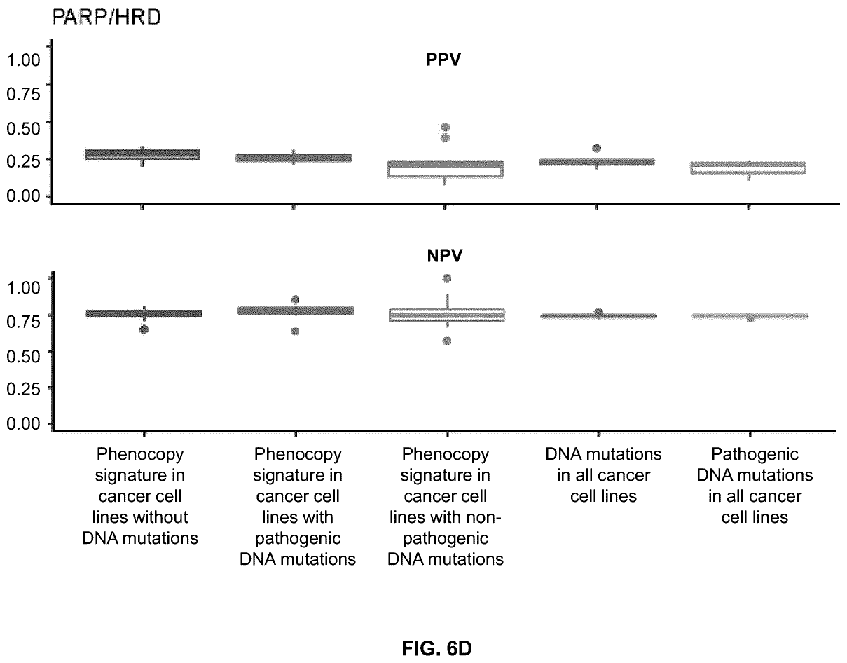

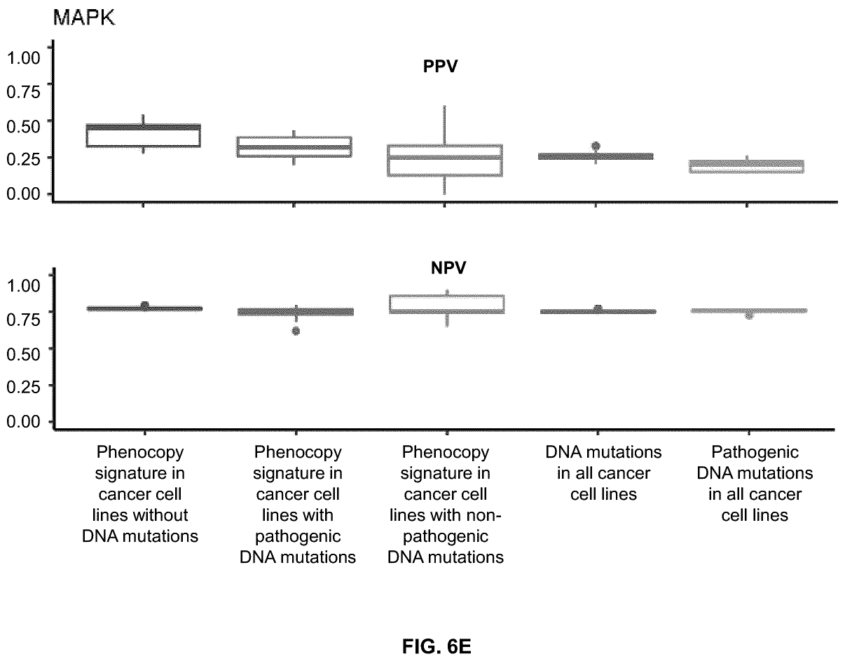

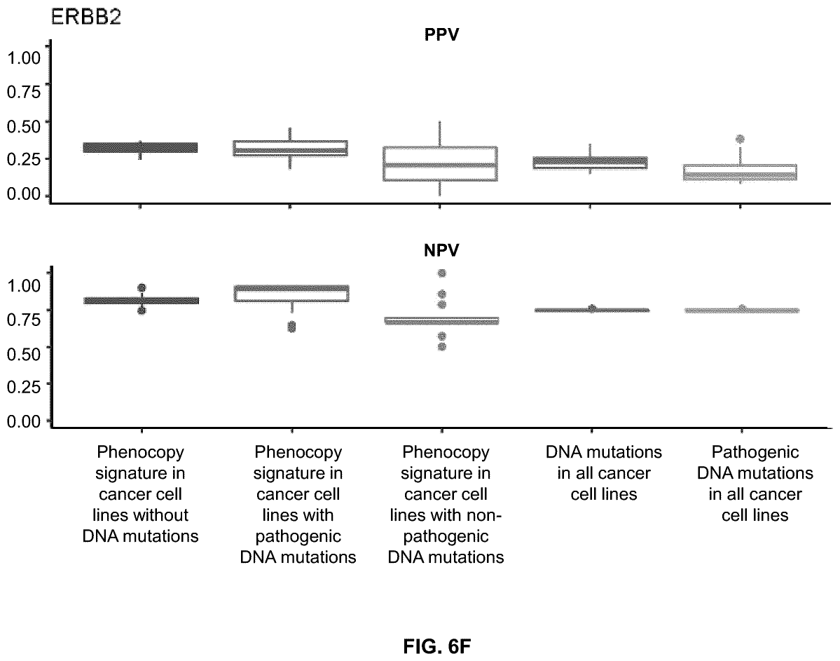

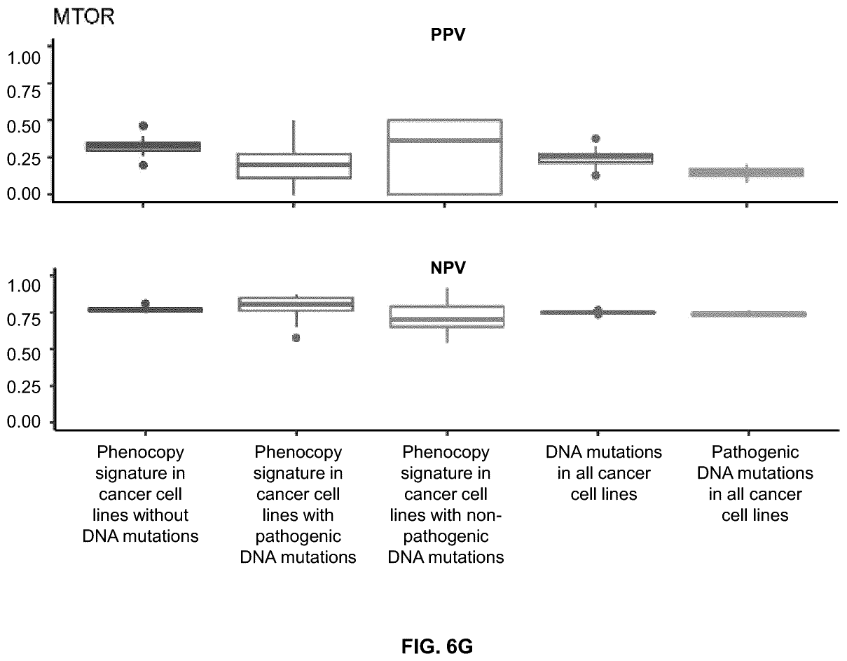

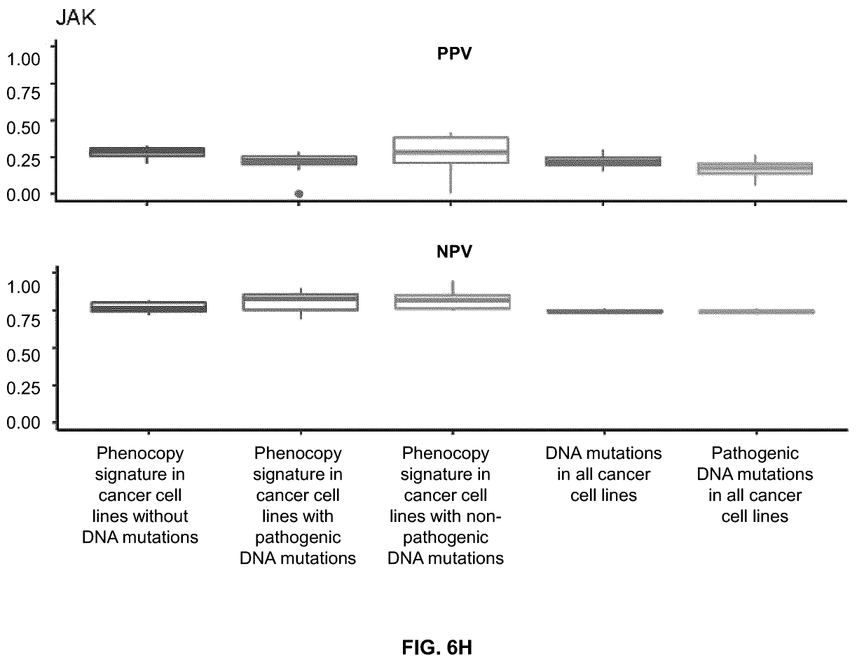

Sensitivity, specificity, positive predictive value (PPV), and negative predictive value (NPV) are commonly used to evaluate clinical biomarkers. In our cell line models, we defined responders as the top quartile. Across all the drugs and pathways tested, 28% of cell lines with a mutation were classified as responders. When limited to pathogenic mutations, this percentage was similar at 26%. In cell lines without a mutation but were predicted to be a phenocopy, a slightly higher 31% were classified as responders, though the sensitivity in individual pathways was frequently higher. The sensitivity of the phenocopy signatures in mutation-negative cancer cell lines was on par with DNA alterations for EGFR, BRAF, and MAPK, and better than DNA alterations for PI3K-AKT, PARP/HRD, ERBB2, MTOR, and JAK. Oncogenic activation of ERBB2 (encoding HER2) in particular is thought to be heavily influenced by amplification, and our results suggest that a mutational phenocopy signature may provide complementary information. The specificity of the phenocopy signatures was high across pathways in identifying responders in cell lines without DNA mutations (FIGS. 5A-5H). The PPVs in cell lines without mutations are almost all improved compared to the results observed with DNA mutations, with the exception of BRAF in which the DNA mutations perform particularly well (FIGS. 6A-6H). As with specificity, the NPVs are high for the phenocopy signatures across groups. These results confirm that the phenocopy signatures are successfully finding additional responders without DNA mutations with high specificity.

While the sensitivity is not as high as the specificity, it is still comparable or better than DNA mutations alone.

### Clinical Validation

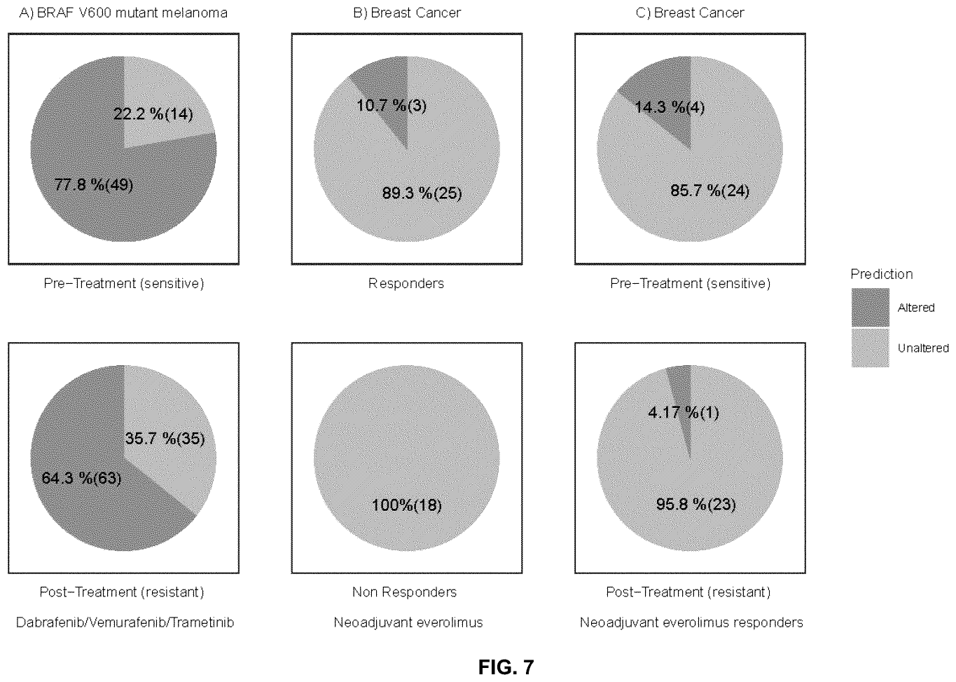

In addition to assessing our model in cell line datasets, we next sought to assess the efficacy of predicting drug response from a phenocopy signature in clinical data. We were able to identify several publicly available clinical cohorts that had treatment response and/or pre/post treatment resistance information for treatments specifically targeting the pathways of our phenocopy signatures. We first examined the BRAF pathway and identified three BRAF-mutant melanoma cohorts with gene expression data (GEO IDs: GSE50509, GSE65185, GSE99898) that were all treated with anti-BRAF therapies (dabrafenib, vemurafenib, trametinib)⁴²⁻⁴⁴. In all three cohorts, pre-treatment (sensitive) and post-treatment (resistant) samples were obtained from the same patients. Because the three cohorts were quite small, and similar in nature, we combined the results of all three. Our normalization approach and phenocopy signatures were applied without modification to each of the three cohorts. Overall, the majority (77.8%) of the pre-treatment (treatment-sensitive) samples were predicted to be BRAF mutation phenocopies, consistent with the fact that all the tumors were known to have BRAF mutations. However, this rate decreased to 64.3% in the post-treatment (treatment-resistant), with a borderline p-value of 0.0806 (FIG. 7, (A)). This is consistent with our in vitro data that the BRAF phenocopy signature predicts response to BRAF inhibitors, as the resistant tumors had a lower rate of phenocopies.

We next identified a cohort of breast cancer patients (GSE119262) that were treated with neoadjuvant everolimus (which targets the mTOR pathway) followed by surgery⁴⁵. In this cohort, both treatment response information and pre-treatment (sensitive) vs. post-treatment (resistant) samples were available. We first examined just the pre-treatment samples. While only a small number were predicted as mTOR mutation phenocopies, 100% of these responded to anti-mTOR therapy compared to 75.8% of the non-mTOR phenocopy tumors (FIG. 7, (B)). When we further examined our phenocopy signature in pre-treatment and post-treatment samples, again none of the non-responder tumors (pre- or post-treatment) were predicted as phenocopies. In the responder tumors, there was a decrease in the rate of phenocopy tumors from 14.3% pre-treatment (sensitive) to 4.17% post-treatment (resistant; FIG. 7, (C)). This is consistent with our in vitro data which demonstrates that a phenocopy signature predicts response to mTOR inhibitors, as the post-treatment resistant tumors had a lower rate of phenocopies.

### Discussion

Targeted therapies have shown great promise in treating a variety of cancer types, but to date only benefit a minority of cancer patients. A major reason is that targeted therapies perform optimally in patients whose specific tumors are uniquely dependent on the targeted pathway, which is currently assessed by identifying key driver mutations. The majority of patients lack a DNA alteration, and we do not currently have other biomarkers to identify additional patients who could benefit from these targeted treatments. With the creation of large pharmacogenomic databases^{2,23,46}, most published efforts have been focused on specifically training molecular signatures to predict drug response²¹⁻³². Our phenocopy approach differs from this direct approach. Instead, we trained phenocopy signatures to identify the gene expression patterns that accompany common driver gene alterations in cancer. We then demonstrate that this indirect approach improves the ability to predict pan-cancer treatment response across eight oncogenic pathways compared to DNA mutation status alone. To our knowledge, this is the first report of the successful global application of a phenocopy strategy in predicting drug response in vitro and in clinical cohorts.

We show that in mutation-negative tumors, the phenocopy signatures can identify a subset that respond to targeted therapies with high specificity. These results suggest that phenocopy signatures add to clinically actionable mutations in predicting therapy response and could be used in clinical settings to identify mutation-negative patients who may benefit from targeted therapy with high specificity. While the sensitivity is not as high, it is comparable to DNA mutations alone and doubling the number of patients eligible for targeted therapies would represent an enormous clinical advancement. Additionally, phenocopy signatures could also be used to help guide treatment decisions for patients with variants of unknown significance. Finally, most drug-biomarker indications are currently limited to specific cancer sites. Our training and validation cohorts are pan-cancer datasets, potentially allowing for a tremendous expansion of current targeted therapy indications across multiple cancer types. Clinical trials or cohorts of targeted therapies with transcriptome-wide RNA profiling are rare. This is partly because most commercial DNA sequencing panels do not include whole-transcriptome RNA-seq. These examples provide a rationale for expanding clinical Next-Gen Sequencing to include RNA-seq, and provide a pan-cancer, platform-independent, phenocopy biomarker with which to select patients for inclusion in a next-generation clinical trial of targeted therapies in patients without driver DNA mutations.

### REFERENCES

1. Liu, S. & Kurzrock, R. Toxicity of targeted therapy: Implications for response and impact of genetic polymorphisms. Cancer Treat Rev 40, 883-891 (2014).

2. Corsello, S. M. et al. Discovering the anti-cancer potential of non-oncology drugs by systematic viability profiling. Nat Cancer 1, 235-248 (2020).

3. Douillard, J.-Y. et al. First-line gefitinib in Caucasian EGFR mutation-positive NSCLC patients: a phase-IV, open-label, single-arm study. Br J Cancer 110, 55-62 (2014).

4. Nan, X., Xie, C., Yu, X. & Liu, J. EGFR TKI as first-line treatment for patients with advanced EGFR mutation-positive non-small-cell lung cancer. Oncotarget 8, 75712-75726 (2017).

5. Kazandjian, D. et al. FDA Approval of Gefitinib for the Treatment of Patients with Metastatic EGFR Mutation-Positive Non-Small Cell Lung Cancer. Clin Cancer Res 22, 1307-1312 (2016).

6. Khozin, S. et al. U.S. Food and Drug Administration approval summary: Erlotinib for the first-line treatment of metastatic non-small cell lung cancer with epidermal growth factor receptor exon 19 deletions or exon 21 (L858R) substitution mutations. Oncologist 19, 774-779 (2014).

7. Khozin, S. et al. Osimertinib for the Treatment of Metastatic EGFR T790M Mutation-Positive Non-Small Cell Lung Cancer. Clin Cancer Res 23, 2131-2135 (2017).

8. Hazarika, M. et al. U.S. FDA Approval Summary: Nivolumab for Treatment of Unresectable or Metastatic Melanoma Following Progression on Ipilimumab. Clin Cancer Res 23,3484-3488 (2017).

9. Kim, G. et al. FDA approval summary: vemurafenib for treatment of unresectable or metastatic melanoma with the BRAFV600E mutation. Clin Cancer Res 20,4994-5000 (2014).

10. Odogwu, L. et al. FDA Approval Summary: Dabrafenib and Trametinib for the Treatment of Metastatic Non-Small Cell Lung Cancers Harboring BRAF V600E Mutations. Oncologist 23,740-745 (2018).

11. Narayan, P. et al. FDA Approval Summary: Alpelisib Plus Fulvestrant for Patients with HR-positive, HER2-negative, PIK3CA-mutated, Advanced or Metastatic Breast Cancer. Clin Cancer Res 27,1842-1849 (2021).

12. Ison, G. et al. FDA Approval Summary: Niraparib for the Maintenance Treatment of Patients with Recurrent Ovarian Cancer in Response to Platinum-Based Chemotherapy. Clin Cancer Res 24,4066-4071 (2018).

13. Anscher, M. S. et al. FDA Approval Summary: Rucaparib for the Treatment of Patients with Deleterious BRCA-Mutated Metastatic Castrate-Resistant Prostate Cancer. Oncologist 26,139-146 (2021).

14. Ascierto, P. A. et al. The role of BRAF V600 mutation in melanoma. J Transl Medi 10, 85 (2012).

15. Kohsaka, S. et al. A method of high-throughput functional evaluation of EGFR gene variants of unknown significance in cancer. Sci Transl Med 9, eaan6566 (2017).

16. Paez, J. G. et al. EGFR Mutations in Lung Cancer: Correlation with Clinical Response to Gefitinib Therapy. Science 304,1497-1500 (2004).

17. Zhang, X.-T. et al. The EGFR mutation and its correlation with response of gefitinib in previously treated Chinese patients with advanced non-small-cell lung cancer. Ann Oncol 16,1334-1342 (2005).

18. Chapman, P. B. et al. Improved survival with vemurafenib in melanoma with BRAF V600E mutation. N Engl J Med 364,2507-2516 (2011).

19. Janku, F. et al. PIK3CA Mutations in Patients with Advanced Cancers Treated with PI3K/AKT/m T OR Axis Inhibitors. Mol Cancer Ther 10,558-565 (2011).

20. Janku, F. et al. PI3K/AKT/mTOR inhibitors in patients with breast and gynecologic malignancies harboring PIK3CA mutations. J Clin Oncol 30, 777-782 (2012).

21. Rydzewski, N. R. et al. Predicting cancer drug TARGETS—TreAtment Response Generalized Elastic-neT Signatures. NPJ Genom Med 6, 76 (2021).

22. Iorio, F. et al. A Landscape of Pharmacogenomic Interactions in Cancer. Cell 166, 740-754 (2016).

23. Barretina, J. et al. The Cancer Cell Line Encyclopedia enables predictive modelling of anticancer drug sensitivity. Nature 483, 603-607 (2012).

24. Polano, M. et al. A Pan-Cancer Approach to Predict Responsiveness to Immune Checkpoint Inhibitors by Machine Learning. Cancers (Basel) 11, E1562 (2019).

25. Reinhold, W. C. et al. Using drug response data to identify molecular effectors, and molecular ‘omic’ data to identify candidate drugs in cancer. Hum Genet 134, 3-11 (2015).

26. Wang, X., Sun, Z., Zimmermann, M. T., Bugrim, A. & Kocher, J.-P. Predict drug sensitivity of cancer cells with pathway activity inference. BMC Med Genomics 12, 15 (2019).

27. Dhruba, S. R., Rahman, R., Matlock, K., Ghosh, S. & Pal, R. Application of transfer learning for cancer drug sensitivity prediction. BMC Bioinformatics 19, 497 (2018).

28. Suphavilai, C., Bertrand, D. & Nagaraj an, N. Predicting Cancer Drug Response using a Recommender System. Bioinformatics 34, 3907-3914 (2018).

29. Wang, L., Li, X., Zhang, L. & Gao, Q. Improved anticancer drug response prediction in cell lines using matrix factorization with similarity regularization. BMC Cancer 17, 513 (2017).

30. Pleasance, E. et al. Pan-cancer analysis of advanced patient tumors reveals interactions between therapy and genomic landscapes. Nat Cancer 1, 452-468 (2020).

31. Sharifi-Noghabi, H., Peng, S., Zolotareva, O., Collins, C. C. & Ester, M. AITL:

Adversarial Inductive Transfer Learning with input and output space adaptation for pharmacogenomics. Bioinformatics 36, i380—i388 (2020).

32. Sharifi-Noghabi, H., Zolotareva, O., Collins, C. C. & Ester, M. MOLI: multi-omics late integration with deep neural networks for drug response prediction. Bioinformatics 35, i501—i509 (2019).

33. Yang, J., Li, A., Li, Y., Guo, X. & Wang, M. A novel approach for drug response prediction in cancer cell lines via network representation learning. Bioinformatics 35, 1527-1535 (2019).

34. Balasubramaniam, S. et al. FDA Approval Summary: Rucaparib for the Treatment of Patients with Deleterious BRCA Mutation-Associated Advanced Ovarian Cancer. Clin Cancer Res 23, 7165-7170 (2017).

35. Alsop, K. et al. BRCA Mutation Frequency and Patterns of Treatment Response in BRCA Mutation—Positive Women With Ovarian Cancer: A Report From the Australian Ovarian Cancer Study Group. J Clin Oncol 30, 2654-2663 (2012).

36. Braicu, C. et al. A Comprehensive Review on MAPK: A Promising Therapeutic Target in Cancer. Cancers (Basel) 11, E1618 (2019).

37. Shin, M. H., Kim, J., Lim, S. A., Kim, J. & Lee, K.-M. Current Insights into Combination Therapies with MAPK Inhibitors and Immune Checkpoint Blockade. Int Mol Sci 21, E2531 (2020).

38. Subramanian, J., Katta, A., Masood, A., Vudem, D. R. & Kancha, R. K. Emergence of ERBB2 Mutation as a Biomarker and an Actionable Target in Solid Cancers. Oncologist 24, e1303—e1314 (2019).

39. Cousin, S. et al. Targeting ERBB2 mutations in solid tumors: biological and clinical implications. J Hematol Oncol 11, 86 (2018).

40. Zou, Z., Tao, T., Li, H. & Zhu, X. mTOR signaling pathway and mTOR inhibitors in cancer: progress and challenges. Cell Biosci 10, 31 (2020).

41. Senkevitch, E. & Durum, S. The promise of Janus kinase inhibitors in the treatment of hematological malignancies. Cytokine 98, 33-41 (2017).

42. Rizos, H. et al. BRAF inhibitor resistance mechanisms in metastatic melanoma: spectrum and clinical impact. Clin Cancer Res 20, 1965-1977 (2014).

43. Hugo, W. et al. Non-genomic and Immune Evolution of Melanoma Acquiring MAPKi Resistance. Cell 162, 1271-1285 (2015).

44. Kakavand, H. et al. PD-L1 Expression and Immune Escape in Melanoma Resistance to MAPK Inhibitors. Clin Cancer Res 23, 6054-6061 (2017).

45. Sabine, V. S. et al. Gene expression profiling of response to mTOR inhibitor everolimus in pre-operatively treated post-menopausal women with oestrogen receptor-positive breast cancer. Breast Cancer Res Treat 122, 419-428 (2010).

46. Yang, W. et al. Genomics of Drug Sensitivity in Cancer (GDSC): a resource for therapeutic biomarker discovery in cancer cells. Nucleic Acids Res 41, D955-961 (2013).

47. Chen, W. S. et al. Novel RB1-Loss Transcriptomic Signature Is Associated with Poor Clinical Outcomes across Cancer Types. Clin Cancer Res 25, 4290-4299 (2019).

48. Way, G. P. et al. Machine Learning Detects Pan-cancer Ras Pathway Activation in The Cancer Genome Atlas. Cell Rep 23, 172-180.e3 (2018).

49. Wang, K., Li, M. & Hakonarson, H. ANNOVAR: functional annotation of genetic variants from high-throughput sequencing data. Nucleic Acids Res 38, e164 (2010).

50. Jassal, B. et al. The reactome pathway knowledgebase. Nucleic Acids Res 48, D498- D503 (2020).

51. Graim, K., Friedl, V., Houlahan, K. E. & Stuart, J. M. PLATYPUS: A Multiple-View Learning Predictive Framework for Cancer Drug Sensitivity Prediction. Pac Symp Biocomput 24, 136-147 (2019).

52. Aggarwal, R. et al. Prognosis Associated With Luminal and Basal Subtypes of Metastatic Prostate Cancer. JAMA Oncology (2021) doi:10.1001/jamaonco1.2021.3987.

### Example 2

### A Phenocopy Signature of TP53 Loss Predicts Response to Chemotherapy

### Summary

Background: TP53 is a the most frequently altered tumor suppressor in cancer. In preclinical studies, p53 protein loss of function has a profound impact on responses to cytotoxic chemotherapies, but this has not been consistently validated clinically. This may be because not all DNA alterations are functional or equal, and conversely, there can be other genomic, epigenomic, or transcriptional changes that produce similar gene expression patterns (i.e. a phenocopy).

Methods: Gene expression-based signatures can improve upon DNA alteration status alone. Therefore, we developed a TP53 loss phenocopy gene expression signature, trained in 9,428 clinical samples from TCGA. We then independently validated that this signature predicted chemotherapy response in three cancer cell line datasets (GDSC, CCLE, DepMap, nearly 1000 cell lines each). Next, we independently validated that the TP53 loss phenocopy signature predicted pathologic response to neoadjuvant chemotherapy in nearly 4000 breast cancer samples from 20 different cohorts/trials.

Results: In vitro, we found that the TP53 loss phenocopy signature predicted chemotherapy response beyond TP53 genomic loss. In a clinical dataset of 3,011 breast cancer samples which were subsequently treated with neoadjuvant chemotherapy, the TP53 loss phenocopy samples were 45% more likely to have a pathologic complete response (pCR, P<0.0001). This difference was present in both ER+ and ER− samples. In the BrighTNess trial, there was a significant association with residual cancer burden continuous score (P=0.005). In time-series data from the I-SPY1 trial, we observed that the proportion of phenocopy samples significantly decreased over the course of neoadjuvant chemotherapy (P=0.0273). Finally, in the I-SPY2 trial (N=987), we again confirmed the overall association with neoadjuvant chemotherapy pCR (P=0.0002). Interestingly, we also observed that the TP53 loss phenocopy tumors were 229% more likely to respond to neoadjuvant chemoimmunotherapy (P=0.004).

Conclusion: The TP53 loss phenocopy signature can clearly predict response to chemotherapy across cancer types in vitro, and response to neoadjuvant chemotherapy clinically in breast cancer. This represents the most extensive validation of a gene expression based TP53 signature for chemotherapy therapy response to date. Furthermore, the dramatic association with chemoimmunotherapy response is the first such report to our knowledge, and has potential clinical implications in both breast and other cancer types.

### Introduction

TP53 is a transcription factor that drives a broad range of anti-proliferative programs in response to diverse intrinsic and extrinsic cellular stressors¹. Consequently, it plays a key tumor suppressor role across tissue types, and is the most commonly altered gene in cancer, with somatic mutations observed in almost all cancer types at rates ranging from 5% to as high as 90%². The spectrum of TP53 somatic mutations is incredibly diverse, with over 2000 different variants described³. About 80% of TP53 mutations occur in the DNA binding domain (DBD), including 6 hotspots that comprise 25% of all TP53 mutations and almost 90% of these DBD mutations are missense mutation that alter DNA binding². Conversely about 60% of mutations outside the DBD are nonsense or truncating mutations². While the majority of TP53 mutations result in loss of the normal function of the protein, some mutations also appear to have dominant-negative and/or gain-of-function oncogenic effects, at least in pre-clinical models⁴. In addition, almost all TP53 mutated tumors exhibit alteration or loss of second allele function through bi-allelic mutation, loss-of-heterozygosity (LOH), or chromosomal deletion⁵. Given this complexity of the TP53 alteration landscape, despite the remarkable depth of pre-clinical data characterizing the impact of diverse TP53 mutations on protein function⁶⁻⁸, it is not possible to interpret the anticipated function of TP53 mutations in patient tumors based on DNA mutation status alone, limiting the development of predictive or prognostic biomarkers and novel targeted therapy approaches based on this frequently mutated and crucial tumor suppressor.

In preclinical studies, p53 protein loss of function has a profound impact on cellular responses to DNA damaging agents including cytotoxic chemotherapies and radiation ⁹⁻¹¹, leading to the hypothesis that TP53 alterations may serve as a biomarker for sensitivity to cytotoxic chemotherapies. Breast cancer has a high frequency of TP53 DNA alterations, which are found in up to 30-40% of cases^{12,13}, and are associated with poor prognosis¹⁴⁻¹⁷, though this is modulated both by TP53 alteration type^{17,18} and breast cancer subtype¹⁹⁻²¹. There has consequently been extensive investigation of p53 status as a biomarker of neoadjuvant chemotherapy response in early-stage breast cancer, with conflicting results. Some smaller cohort studies have found an association between TP53 DNA alterations and chemotherapy response²²⁻²⁴, while others have found the inverse^{17,25,26}. Likewise, while some smaller series found an association between low p53 protein expression and chemotherapy response²⁷, others associated p53 overexpression with chemotherapy response^{22,28}, and several found no association between p53 protein and response^{23,29}. In many cases, these studies may have been confounded by heterogeneity in the spectrum of detected TP53 alterations as well as heterogeneity in breast cancer subtype, which impacts both frequency and type of TP53 alteration and neoadjuvant therapy response rates. However, TP53 DNA alteration status as also been evaluated as a biomarker of breast cancer neoadjuvant chemotherapy response in two large clinical trial cohorts: EORTC 10944/BIG 1-00³⁰, a phase III trial comparing anthracycline versus anthracycline/taxane neoadjuvant therapy across breast cancer subtypes and the GeparSixto phase II trial³¹ evaluating anthracycline/taxane/platinum based neoadjuvant therapy in triple negative and HER2-positive breast cancer. Neither of these trials found any association between TP53 DNA alteration status and neoadjuvant chemotherapy response.

DNA alterations can create upstream and downstream changes in gene expression throughout a pathway. However, not all DNA alterations are functional, and conversely, there can be other genomic, epigenomic, or transcriptional changes that produce a similar gene expression pattern (i.e. a phenocopy). Gene expression-based signatures can often improve on DNA alteration status alone. We have created RNA-based phenocopy signatures for multiple DNA alterations (other than TP53) that serve as indications for targeted therapy and demonstrated that the phenocopy signatures added to the DNA alterations in predicting treatment response to those same targeted agents³³ (Example 1). Herein, we used this same approach to develop a new RNA-based phenocopy signature for TP53 loss as a predictor for cytotoxic chemotherapy response.

### Methods

### Datasets and Availability

As in Example 1 (see also³³), we utilized TCGA as our training dataset and GDSC, CCLE, and DepMap as our in vitro validation datasets. Processed DNA and RNA sequencing data from TCGA were downloaded using the UCSC Xena browser (xena.ucsc.edu). Processed DNA/RNA sequencing data and chemotherapy response data for the Genomic of Drug Sensitivity in Cancer (GDSC) dataset were downloaded from the GDSC website (www.cancerrxgene.org). Processed DNA/RNA sequencing data and chemotherapy response data for the Cancer Cell Line Encyclopedia (CCLE) were downloaded from the CCLE website (portals.broadinstitute.org/ccle). The Cancer Dependency Map (DepMap) shares the same cell lines and therefore DNA/RNA sequencing data as CCLE, but independently evaluated chemotherapy response, which was obtained from the DepMap website (depmap.org). As recommended by DepMap, the MTS010 dataset was used for drug response data.

In addition, we sought to identify published cohorts with pre-chemotherapy RNA profiling with pathologic response data. The majority of data we could find was in the setting of breast cancer neoadjuvant chemotherapy, where response could be assessed at the time of surgery. Our dataset evaluating clinical treatment response and the phenocopy signatures in breast cancer included 18 breast cancer cohorts with gene expression data downloaded from the Gene Expression Omnibus (GEO) with the following accession numbers: GSE22093, GSE25055, GSE41998, GSE20194, GSE4779, GSE8465, GSE66399, GSE16446, GSE20271, GSE18864, GSE25065, GSE32646, GSE164458, GSE22226, GSE192341, GSE163882, GSE34138, GSE32603, and one additional publicly available TNBC gene expression cohort⁴⁸. Finally, we utilized gene expression and clinical response data from the I-SPY2 trial as an independent clinical validation cohort for our phenocopy signatures and breast cancer neoadjuvant therapy response, also downloaded from GEO (GSE194040).

### TP53 Loss Phenocopy RNA Signature Development

We wanted to focus on TP53 alterations leading to the tumor suppressor loss. The presence of two DNA alterations in TP53 most likely represents bi-allelic loss-of-function. Therefore, we defined TP53 DNA loss as either two TP53 coding mutations, or a single coding mutation and CN loss. While some single TP53 alteration samples may have a tumor suppressor phenotype through LOH or a dominant negative phenotype, specificity in training the model was prioritized, and thus using only on the most confident examples of TP53 loss-of-function was more important than sensitively identifying all examples. In addition, MDM2 is an E3 ubiquitin ligase that plays a key role in regulating p53 through proteasomal degradation, can also lead to loss of wild type p53 activity, mimicking the tumor promoting phenotype of TP53 genomic loss of function⁴⁹, and we also included alteration in MDM2 as previously described³³. This set of alterations is referred to as “TP53 loss” throughout the present examples. Likewise, we utilized the public calls for mutation and CN in TCGA, GDSC, and CCLE/DepMap without modification³³. For TCGA, a GISTIC threshold of −1 was used for CN loss. GDSC had CN loss pre-defined in the downloaded data, which is what we used. In CCLE, CN loss was defined as a Log₂ CN<−1. We again utilized the Reactome database to identify a list of genes associated with TP53 status with which to train our RNA phenocopy model. We selected the “TP53 Regulates Transcription of Cell Cycle Genes” pathway as transcription is what we are measuring, and the cell cycle pathway is the target of cytotoxic chemotherapies. The genes in this pathway are: CNOT4, RBL1, BAX, AURKA, CNOT3, PCBP4, EP300, E2F1, RGCC, RBL2, CCNE1, CDKN1B, CNOT2, CNOT6, TFDP2, ARID3A, GADD45A, PLAGL1, CDK2, CDKN1A, CNOT1, PRMT1, E2F8, PCNA, CCNA1, CCNB1, CNOT6L, TP53, CARM1, CCNA2, PLK2, TNKS1BP1, CENPJ, CNOT8, CDC25C, CNOT11, BTG2, ZNF385A, E2F7, CDK1, PLK3, CCNE2, SFN, NPM1, CNOT10, TFDP1, CNOT7, and E2F4. We then trained a gradient boosted tree (XGBoost) model using TCGA pan-cancer samples, similar to our prior efforts (Example 1)³³.

### Gene Expression Normalization and Batch Correction

Given the diversity of gene expression platforms across in vitro and clinical validation datasets, we performed batch correction and normalization using the same method as previously described^{33,50}. In brief, every sample was rank normalized with a ‘dense’ method for ties. For GDSC and CCLE/DepMap we then performed batch correction using the R SVA package (COMBAT) across all cancer types with TCGA as the reference (as these datasets have a large number of cancer types). For the breast cancer datasets, we performed the COMBAT batch correction step only against ‘breast invasive carcinoma’ (BRCA) samples within TCGA. There was no missing data in GDSC or CCLE/DepMap. Some of the breast cancer clinical studies had gene expression data missing for a small number of genes in certain samples. In these cases, we imputed the average gene expression for that gene across all the other samples in that cohort.

### Independent Validation In Vitro

We next validated our model in the GDSC, CCLE, and DepMap cell line databases. GDSC had both DNA/RNA data, but CCLE and DepMap shared DNA/RNA data with independent drug response assessments. We identified all cytotoxic chemotherapies assessed in these cohorts. The TP53 phenocopy signature was then applied without modification to the GDSC and CCLE/DepMap, with normalization/batch correction as above. Although there is some overlap between chemotherapeutic agents used in each cohort, we chose to analyze each cohort independently as each study was done separately in a manner as previously described³³. Briefly, to assess if the phenocopy signature predictions were associated with chemotherapy response, we created a linear model for each drug with the dependent variable as a measurement of drug sensitivity. This was the Z-score of the IC50 in GDSC, -ActArea in CCLE, and -AUC in DepMap, such that a lower score was associated with increased drug sensitivity³³. The independent variables in the linear model were then the TP53 RNA phenocopy score, and DNA TP53 Loss as defined above.

### Breast Cancer Clinical Validation

Each of the breast cancer datasets had annotations for pathologic response (pCR versus no pCR) for each sample. The TP53 phenocopy signature was then applied without modification to pre-treatment samples from each breast cancer study, with normalization/batch correction as above. We then compared the responder (pCR)/non-responder (no pCR) proportions in the phenocopy vs. not phenocopy groups using Fisher's exact test. The GSE164458 cohort from the phase III BrighTNess trial³⁸ in triple negative breast cancer was also annotated for residual cancer burden (RCB) status in patients with no pCR, and proportion of each RCB class in phenocopy versus not phenocopy groups was compared using a Cochrane-Armitage test. Furthermore, the GSE32603 cohort from the I-SPY1 trial³⁹ contained pre-treatment, on-treatment (24-72 h), and post-treatment (surgery) samples, allowing us to assess how the phenocopy predictions changed longitudinally, with significance of change in proportion of pCR/no pCR samples at each timepoint compared using a Cochrane-Armitage test. The GSE194040 cohort from the I-SPY2 clinical trial³⁶ was used as an independent clinical validation set, and to evaluate the association between the TP53 phenocopy signature and chemoimmunotherapy response.

### Results

### TP53 Loss Phenocopy Model Development

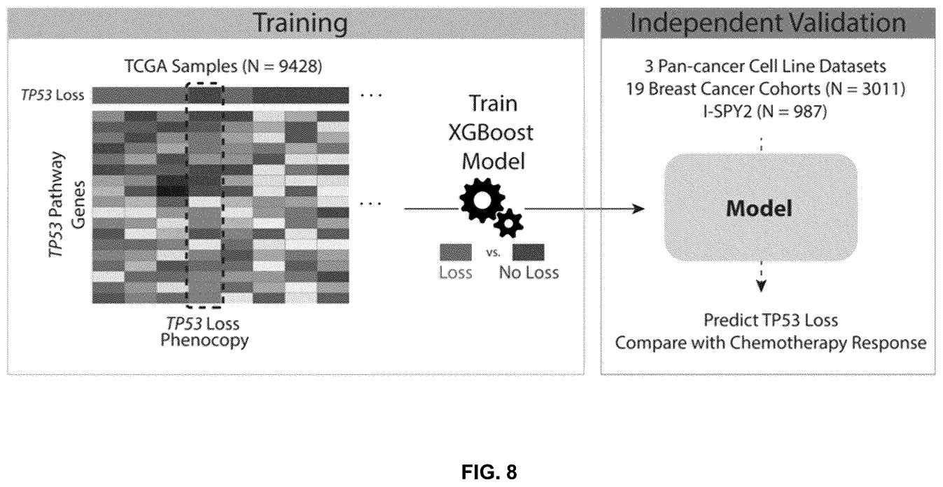

Using the same machine learning methodology used for previously published phenocopy signatures, we trained and locked a TP53 loss phenocopy model in 9,428 clinical samples from the Cancer Genome Atlas (TCGA) as described in the methods (FIG. 8). While the TCGA training data did not have chemotherapy treatment response data, we independently validated that the TP53 loss signature predicts for sensitivity to chemotherapy in three pan-cancer cell line datasets (GDSC, CCLE, DepMap). Finally, we independently validated that our signature predicted pathologic response to neoadjuvant chemotherapy in 19 breast cancer clinical datasets (N=3,011) as well as the I-SPY2 adaptive trial (N=987).

### TP53 Loss Phenocopies are Enriched for DNA Alterations

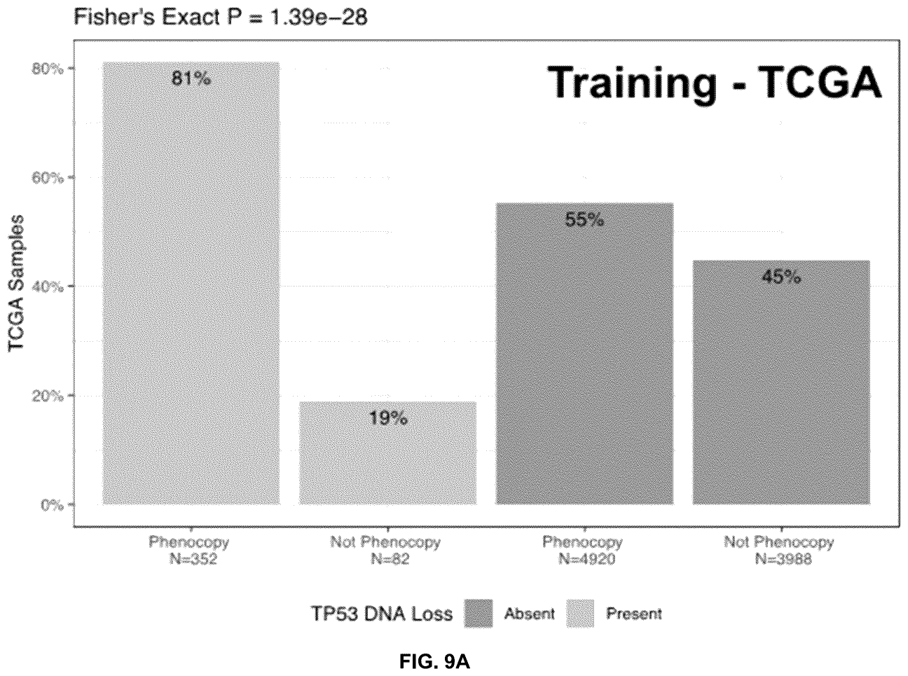

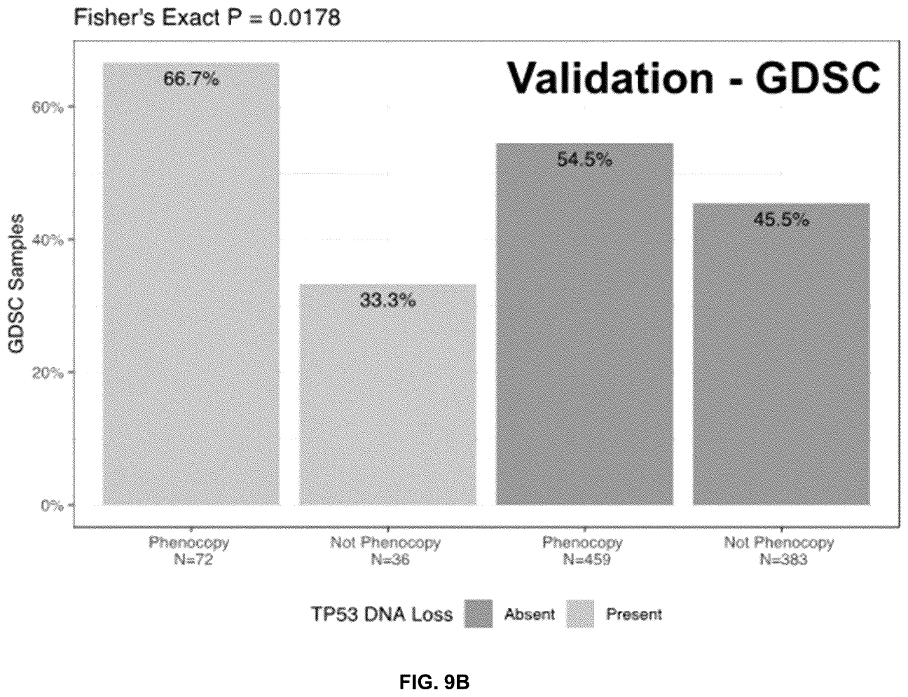

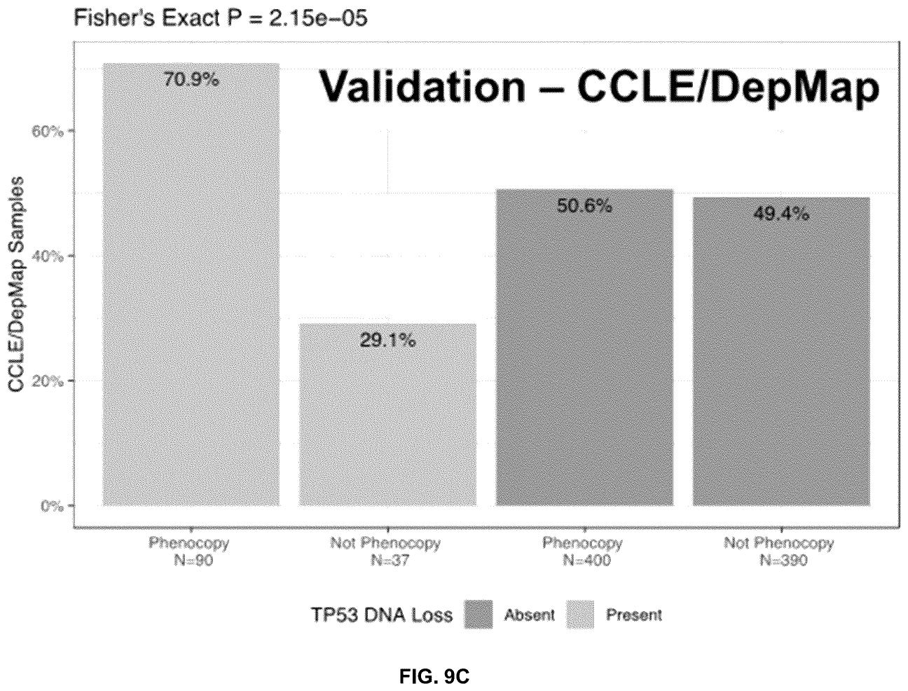

First, we examined the association between the TP53 loss phenocopy signature and the DNA alterations used to train the model. We anticipated that phenocopy positive samples would include those with a TP53-loss genotype as defined in our training cohort, as well as samples without the TP53-loss genotype but with other genomic or epigenomic alterations leading to impaired TP53 function and/or a functional TP53 loss-like phenotype. Indeed, in the TCGA training cohort, (N=9,428), we found that 81% of the TP53 loss samples were predicted to be a phenocopy compared to 55% of the non-TP53 loss samples (Fisher's Exact P<0.0001; FIG. 9A). We next examined our in vitro validation cohorts, which also had both DNA alteration and gene expression information. Similar to TCGA, in GDSC (N=950), 66.7% of the TP53 loss samples were predicted to be a phenocopy compared to 54.5% of the non-TP53 loss samples (Fisher's Exact P=0.0178; FIG. 9B). Likewise, in CCLE/DepMap (which share NGS data; N=917), 70.9% of the TP53 loss samples were predicted to be a phenocopy compared to 50.6% of the non-TP53 loss samples (Fisher's Exact P<0.0001; FIG. 9C). Thus, while most of the samples with genomic TP53 loss were predicted phenocopies, as expected a relatively high number of phenocopies were also predicted in the non-TP53 loss group. This highlights the possibility that a gene expression-based phenocopy definition of TP53 loss could identify functional p53 pathway impairment effectively and perhaps more broadly than genotype-based definitions.

TP53 Loss Phenocopy Status Predicts Response to Chemotherapy across 3 In Vitro Studies

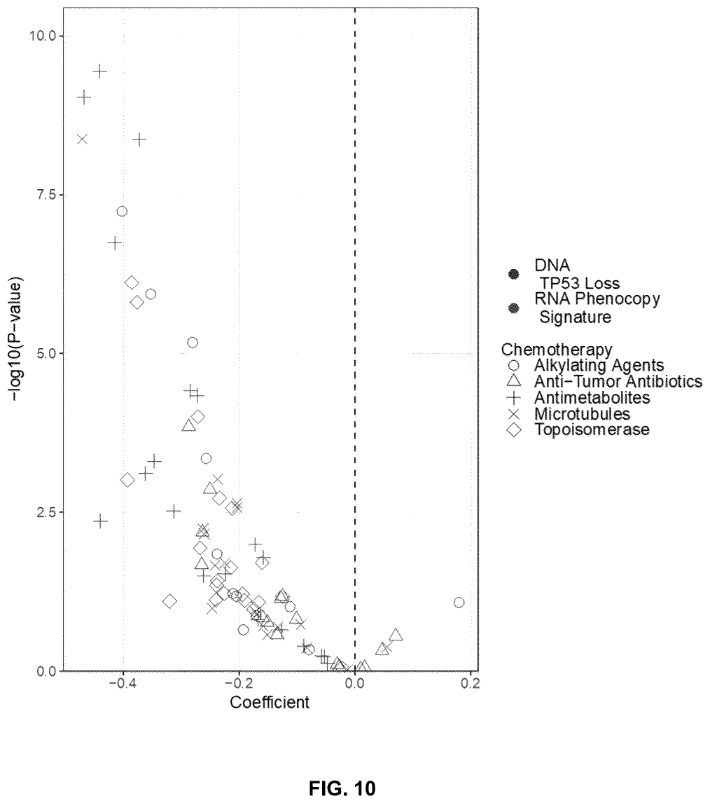

We next sought to determine if our TP53 phenocopy signature predicts response to cytotoxic chemotherapies. We utilized the three cell line datasets (GDSC, CCLE and DepMap) that contain response data for many cytotoxic chemotherapies across large panels of cancer cell lines annotated with both genomic and gene expression profiling. To ask whether TP53 loss phenocopy status predicts chemotherapy response, we created linear models with either the RNA phenocopy signatures or DNA TP53 loss status predicting response for each chemotherapy. We found that across most cytotoxic chemotherapies in all three datasets, the phenocopy signatures significantly predicted chemotherapy response even when accounting for DNA TP53 loss status (FIG. 10). This pan-cancer in vitro validation of this signature to predict chemotherapy response beyond DNA alterations alone supports further investigation in clinical datasets.

TP53 Loss Phenocopy Status Predicts Pathologic Complete Response to Neoadjuvant Chemotherapy across 19 Breast Cancer Clinical Studies

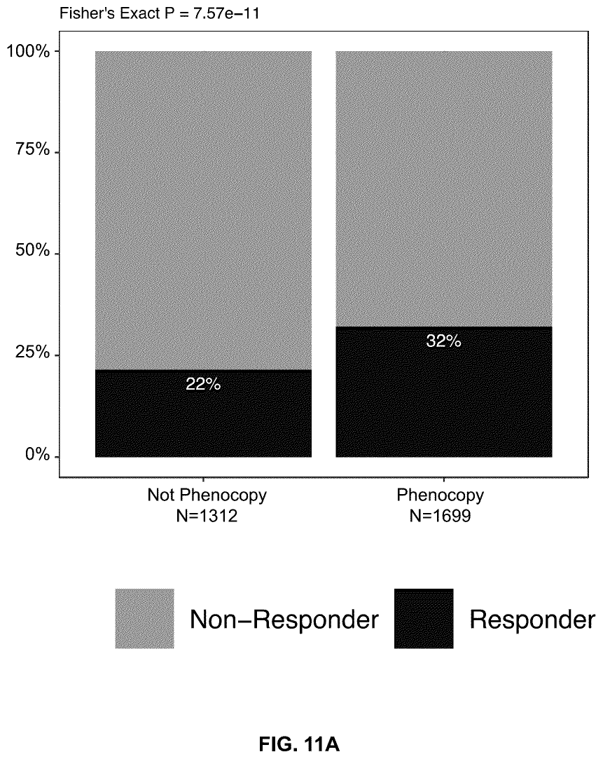

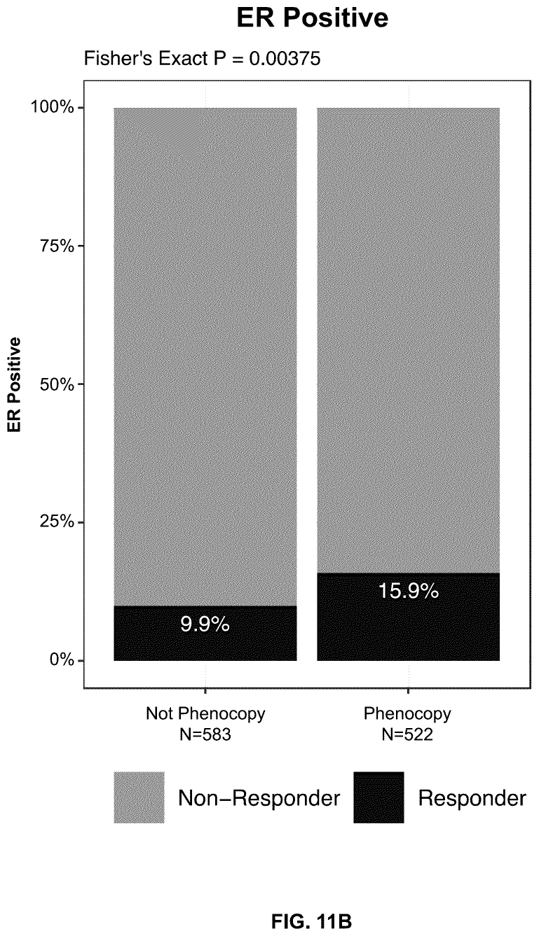

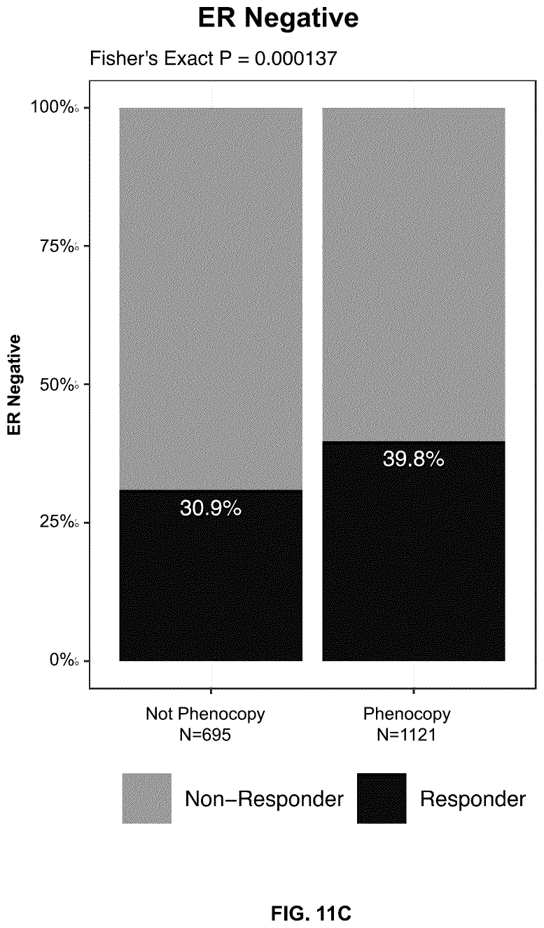

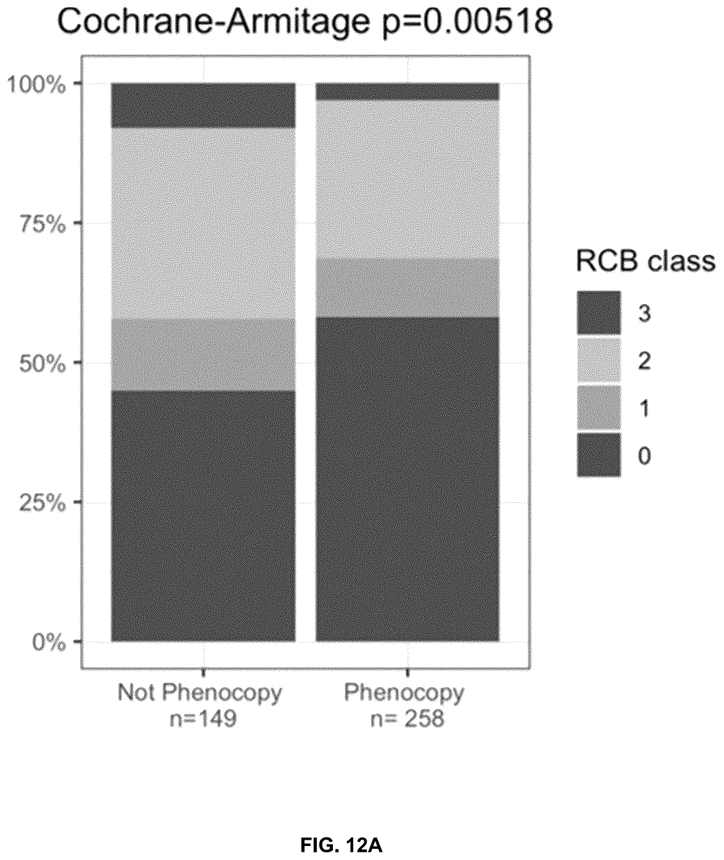

Validation in clinical samples is critical for any molecular predictor of response. Neoadjuvant chemotherapy is an important component of treatment for early-stage breast cancer³⁴, particularly the HER2-positive and triple negative subtypes where pathologic response to neoadjuvant chemotherapy is strongly associated with prognosis and recurrence risk³⁵. There are currently no tools for the prediction of response to neoadjuvant chemotherapy, which could allow for individualized tailoring of chemotherapy intensity with treatment escalation and de-escalation approaches. Given the convincing in vitro results, we next sought to investigate the TP53 loss phenocopy signature and pathologic complete response after neoadjuvant chemotherapy in early-stage breast cancer, whereas genomic TP53 loss has not reliably predicted response^{30,31}. To address this question, we examined 19 breast cancer datasets comprising a total of 3,011 samples with pre-treatment gene expression profiling and clinical pathologic complete response annotation. All patients were treated with neoadjuvant cytotoxic chemotherapy following tissue profiling, and pathologic response assessment was performed at the time of surgery. In total, we found that 32% of the phenocopy samples had a pathologic complete response (pCR), compared to 22% of the non-phenocopy samples (Fisher's Exact P<0.0001, FIG. 11A).

While recurrent TP53 DNA alterations are relatively common in breast cancer, ER-positive breast cancers have much lower rates of TP53 DNA alterations than ER-negative (HER2 positive and triple negative) tumors¹³. Additionally, ER-positive tumors have much lower rates of pCR after neoadjuvant chemotherapy than ER-negative tumors reflecting relative chemoresistance of the ER-positive subtype³⁶. We found that RNA phenocopy signature rates differed by ER status, with 47.2% of ER-positive samples compared to 61.7% of the ER-negative samples predicted TP53 phenocopies (FIGS. 11B and 11C). Intriguingly, while overall rates of pCR were lower for ER-positive cancers as expected, there was still a significant association between phenocopy status and likelihood of pCR, seen in 15.9% of the phenocopy samples compared to 9.9% of the non-phenocopy samples in ER-positive disease (Fisher's Exact P<0.0001, FIG. 11B). Likewise, for ER-negative disease, 39.8% of the phenocopy samples had a pCR, compared to 30.9% of the non-phenocopy samples (Fisher's Exact P<0.0001, FIG. 11C). Taken together, the combination of the pan-cancer in vitro and breast cancer clinical results provide clear evidence that the RNA TP53 phenocopy signature is associated with chemotherapy response.

### TP53 Loss Phenocopy Status Predicts Residual Disease Burden in Triple Negative Breast Cancer in the BrighTNess Phase III Trial

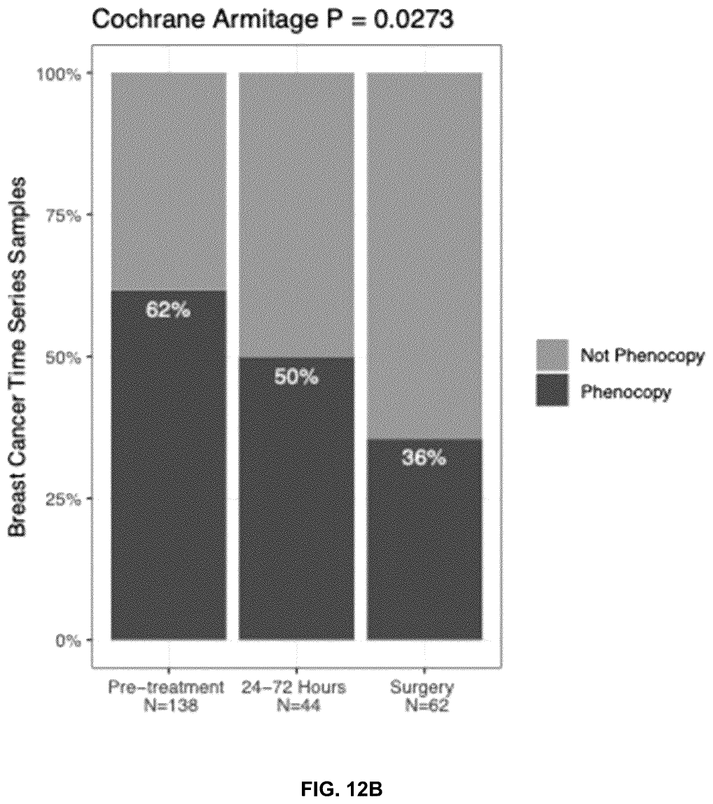

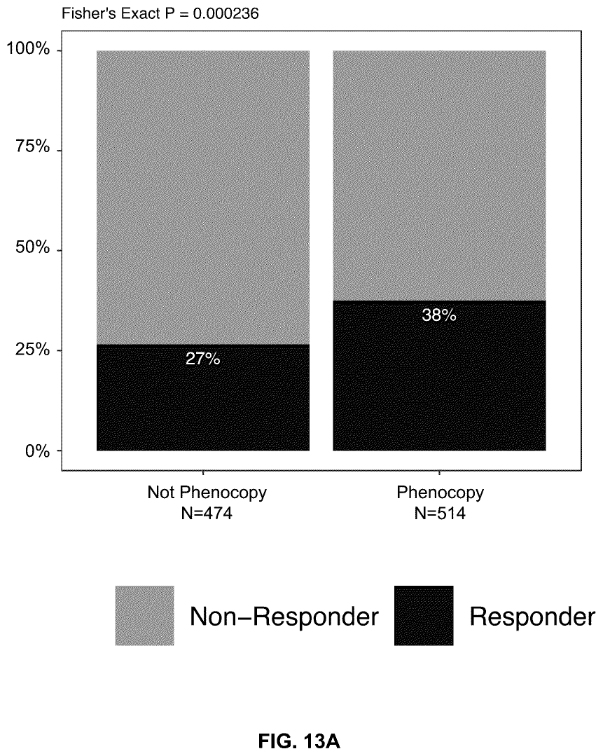

Despite recent treatment advances, early-stage triple negative breast cancers continue to have a worse prognosis than other breast cancer subtypes. In patients with residual disease after neoadjuvant chemotherapy, the pathologic residual cancer burden (RCB) method to quantify extent of residual disease has been shown to have significant prognostic value in stratifying recurrence risk³⁷. This method classifies residual disease status as RCB-0 (pCR), RCB-I, RCB-II, and RCB-III based on standardized pathologic parameters, with higher class indicative of greater extent of residual disease and associated with higher recurrence risk. We leveraged gene expression samples from the large phase III BrighTNess clinical trial of neoadjuvant anthracycline/taxane therapy+/−platinum and PARP inhibitor veliparib in triple negative breast cancer³⁸ for which RCB class annotation was available to evaluate the association between pre-treatment phenocopy status and RCB class. We observed that there was a significantly lower burden of residual disease across RCB categories in the phenocopy group compared to the not phenocopy group (Cochrane-Armitage P=0.00518, FIG. 12A), consistent with a continuous association of TP53 phenocopy status with magnitude of chemotherapy response.

### TP53 Loss Phenocopy Proportion Decreases During Course of Chemotherapy in the I-SPY1 Trial

In addition, one of the clinical breast cancer cohorts collected time-series data before, during, and after treatment with neo-adjuvant chemotherapy from the I-SPY1 trial³⁹. We examined the proportion of samples predicted as phenocopies at each time point. Pre-treatment, 62% of samples were predicted as phenocopies. However, this decreased to 50% by 24-72 hours on-treatment, and further decreased to 36% by the end of chemotherapy on the surgical specimen (Cochrane-Armitage P=0.0273, FIG. 12B). If our TP53 phenocopy indeed predicts chemo-sensitivity as our in vitro and clinical data suggest, then we would expect that chemotherapy would cause a reduction in phenocopy tumor cells as the most sensitive cells are killed, matching our observation in this dataset.

### TP53 Loss Phenocopy Status Predict Response to Neoadjuvant Chemo-immunotherapy in Triple Negative Breast Cancer in the I-SPY2 Adaptive Trial

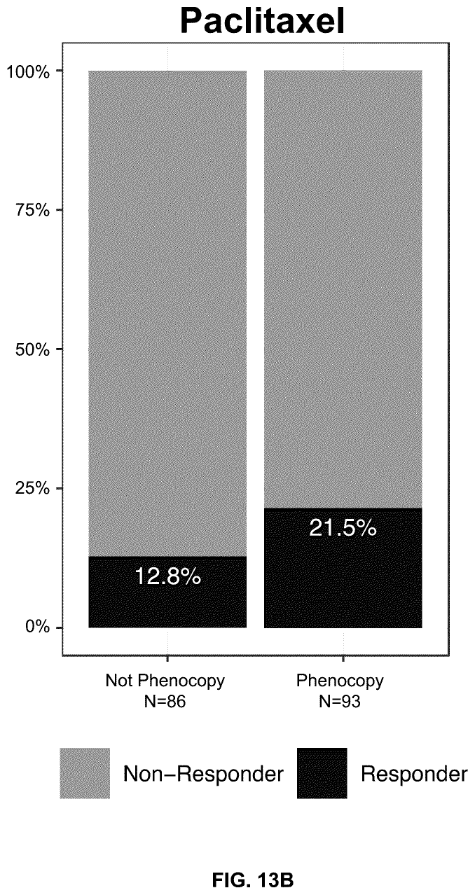

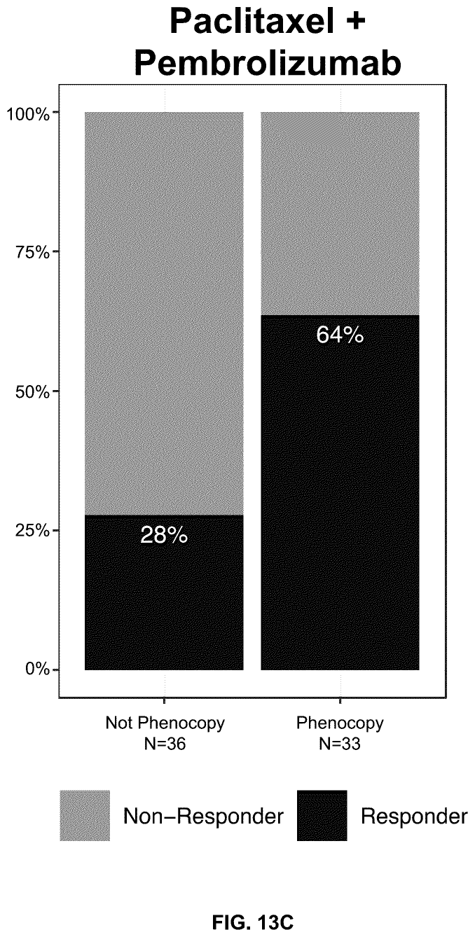

Chemotherapy plus pembrolizumab has become the standard of care for most triple negative breast cancers after the demonstration of both increased pCR rates and improved event free survival with the addition of pembrolizumab to chemotherapy in the randomized phase III Keynote 522 trial⁴⁰. We therefore wanted to validate that the TP53 loss phenocopy signature would still predict response to this new regimen. We therefore leveraged the I-SPY2 990 Data Resource³⁶, comprising gene expression and clinical response data from 987 patients enrolled in the I-SPY2 adaptive neoadjuvant trial platform with an anthracycline/taxane-based neoadjuvant chemotherapy backbone. As expected, TP53 phenocopy status was also significantly associated with pathologic complete response overall in I-SPY2, with a 38% pCR rate in the phenocopy samples versus 27% of non-phenocopy samples (Fisher's Exact P=0.000236, FIG. 13A). One arm of I-SPY2 was treated with chemoimmunotherapy (with pembrolizumab). Concordant with the subsequent findings from the Keynote 522 trial, the pCR rate was overall higher in this arm (FIG. 13C) compared to the chemotherapy only control arm (FIG. 13B). However, while the pCR rates and delta between phenocopy and non-phenocopy groups were similar in the chemotherapy only arm (FIG. 13B) to what we observed in our prior aggregate chemotherapy response dataset (FIGS. 11A-11C), the pCR rate in the chemoimmunotherapy phenocopy samples as well as the delta between phenocopy and non-phenocopy samples was markedly and disproportionately higher at 64% in the phenocopy samples versus 28% in the non-phenocopy samples (FIG. 13C; Fisher's Exact=0.00376). This intriguing finding raises the possibility that TP53 functional status may be a biomarker of response to neoadjuvant chemoimmunotherapy and could play a role in guiding treatment escalation and de-escalation in triple negative breast cancer.

### Discussion

In the present example, we describe a pan-cancer phenocopy signature for TP53 loss trained in a cohort of 9,428 clinical samples from the Cancer Genome Atlas (TCGA). We then validate that the TP53 phenocopy signature predicts for sensitivity to chemotherapy beyond DNA TP53 mutation status and across tumor types in three large cell line databases. To validate this finding clinically, we aggregated 3,011 breast cancer biopsy samples across 19 cohorts and identified a significant association between our TP53 phenocopy signature and pathologic complete response and extent of residual disease after neoadjuvant chemotherapy in early-stage breast cancer. Finally, we performed further independent clinical validation in the I-SPY2 breast cancer neoadjuvant chemotherapy adaptive trial (N=987) confirming the significant association between TP53 phenocopy status and pathologic complete response, as well as identifying an even stronger association with chemoimmunotherapy.

To our knowledge, this represents the most extensive clinical validation of a gene expression based TP53 signature in breast cancer neoadjuvant therapy response to date. In contrast to prior studies focused on DNA alteration status that had very mixed results^{30,31} we have been able to develop a biomarker robustly associated with chemosensitivity by focusing on a transcriptional profile most consistent with TP53 loss of function. Importantly, we also identified a significant association between our TP53 phenocopy status and residual disease burden after standard neoadjuvant chemotherapy, suggesting that our signature is continuously associated with degree of chemosensitivity, and as such could play a role in guiding escalation and de-escalation of therapy at a more granular level.

Intriguingly, we saw a particularly strong association between TP53 phenocopy status and chemoimmunotherapy response in triple negative breast cancer. The impact of tumor cell intrinsic TP53 alterations on anti-tumor immune responses is complex⁴², however TP53 mutations have been associated with increased response to chemoimmunotherapy in non-small cell lung cancer⁴³ and mutated TP53 has been shown to induce anti-tumor T cell responses as a neoantigen in ovarian cancer^{44,45}, and it is possible that similar mechanisms play a role in chemoimmunotherapy response in triple negative breast cancer which has the highest rate of TP53 mutations across breast cancer subtypes¹⁹. From a clinical perspective, biomarkers to guide escalation and de-escalation of neoadjuvant chemoimmunotherapy approaches in triple negative breast cancer would be immensely useful. The TP53 phenocopy signature represents the first such biomarker that we are aware of.

Chemosensitivity is a complex phenotype that can be mediated by multiple factors beyond p53 function, including upregulation of drug efflux pumps, intracellular drug metabolism pathways, increased DNA repair pathway activity and reduced apoptosis⁴⁶ as well as changes in the tumor microenvironment and tumor-immune interface⁴⁷. While TP53 functional status is a component, this complexity is reflected in the modest effect size that we observed in our clinical validation. However, the TP53 phenocopy signature is clearly associated with neoadjuvant chemotherapy response in breast cancer across multiple clinical cohorts comprising nearly 4000 patients, as well as even more strongly associated with chemoimmunotherapy response. However, given that phenocopy signature was trained on clinical samples across cancer types and validated in pan-cancer in vitro datasets for chemotherapy response, this potentially supports broader applicability across tumor types.

### REFERENCES

1. Levine AJ. p53: 800 million years of evolution and 40 years of discovery. Nat Rev Cancer. 2020;20(8):471-80.

2. Olivier M, Hollstein M, and Hainaut P. TP53 mutations in human cancers: origins, consequences, and clinical use. Cold Spring Harb Perspect Biol. 2010;2(1):a001008.

3. Leroy B, Anderson M, and Soussi T. TP53 mutations in human cancer: database reassessment and prospects for the next decade. Hum Mutat. 2014;35(6):672-88.

4. Soussi T, and Wiman KG. TP53: an oncogene in disguise. Cell Death Differ. 2015;22(8): 1239-49.

5. Donehower LA, Soussi T, Korkut A, Liu Y, Schultz A, Cardenas M, et al. Integrated Analysis of TP53 Gene and Pathway Alterations in The Cancer Genome Atlas. Cell Rep. 2019;28(5):1370-84 e5.

6. de Andrade KC, Lee EE, Tookmanian EM, Kesserwan CA, Manfredi JJ, Hatton JN, et al. The TP53 Database: transition from the International Agency for Research on Cancer to the US National Cancer Institute. Cell Death Differ. 2022;29(5):1071-3.

7. Kennedy MC, and Lowe SW. Mutant p53: it's not all one and the same. Cell Death Differ. 2022;29(5): 983 -7.

8. Kato S, Han SY, Liu W, Otsuka K, Shibata H, Kanamaru R, et al. Understanding the function-structure and function-mutation relationships of p53 tumor suppressor protein by high-resolution missense mutation analysis. Proc Natl Acad Sci USA. 2003 ; 100(14): 8424-9.

9. Lowe SW, Ruley HE, Jacks T, and Housman DE. p53-dependent apoptosis modulates the cytotoxicity of anticancer agents. Cell. 1993;74(6):957-67.

10. Lowe SW, Bodis S, McClatchey A, Remington L, Ruley HE, Fisher DE, et al. p53 status and the efficacy of cancer therapy in vivo. Science. 1994;266(5186):807-10.

11. Bunz F, Hwang PM, Torrance C, Waldman T, Zhang Y, Dillehay L, et al. Disruption of p53 in human cancer cells alters the responses to therapeutic agents. J Clin Invest. 1999;104(3):263-9.

12. Curtis C, Shah SP, Chin SF, Turashvili G, Rueda OM, Dunning MJ, et al. The genomic and transcriptomic architecture of 2,000 breast tumours reveals novel subgroups. Nature. 2012;486(7403): 346-52 .

13. Network CGA, Getz G, Chin L, Mills GB, and Ingle JN. Comprehensive molecular portraits of human breast tumours. Nature. 2012;490(7418):61-70.

14. Pharoah PD, Day NE, and Caldas C. Somatic mutations in the p53 gene and prognosis in breast cancer: a meta-analysis. Br. J Cancer. 1999;80(12):1968-73.

15. Meric-Bernstam F, Zheng X, Shariati M, Damodaran S, Wathoo C, Brusco L, et al. Survival Outcomes by TP53 Mutation Status in Metastatic Breast Cancer. JCO Precis Oncol. 2018;2018.

16. Blaszyk H, Hartmann A, Cunningham JM, Schaid D, Wold LE, Kovach JS, et al. A prospective trial of midwest breast cancer patients: a p53 gene mutation is the most important predictor of adverse outcome. Int J Cancer. 2000;89(1):32-8.

17. Dobes P, Podhorec J, Coufal O, Jureckova A, Petrakova K, Vojtesek B, et al. Influence of mutation type on prognostic and predictive values of TP53 status in primary breast cancer patients. Oncol Rep. 2014;32(4):1695-702.

18. Powell B, Soong R, Iacopetta B, Seshadri R, and Smith DR. Prognostic significance of mutations to different structural and functional regions of the p53 gene in breast cancer. Clin Cancer Res. 2000;6(2): 443 -51.

19. Silwal-Pandit L, Vollan HK, Chin SF, Rueda OM, McKinney S, Osako T, et al. TP53 mutation spectrum in breast cancer is subtype specific and has distinct prognostic relevance. Clin Cancer Res. 2014;20(13):3569-80.

20. Coates AS, Millar EK, O'Toole SA, Molloy TJ, Viale G, Goldhirsch A, et al. Prognostic interaction between expression of p53 and estrogen receptor in patients with node-negative breast cancer: results from IBCSG Trials VIII and IX. Breast Cancer Res. 2012; 14(6):R143.

21. Coutant C, Rouzier R, Qi Y, Lehmann-Che J, Bianchini G, Iwamoto T, et al. Distinct p53 gene signatures are needed to predict prognosis and response to chemotherapy in ER-positive and ER-negative breast cancers. Clin Cancer Res. 2011;17(8):2591-601.

22. Kandioler-Eckersberger D, Ludwig C, Rudas M, Kappel S, Janschek E, Wenzel C, et al. TP53 mutation and p53 overexpression for prediction of response to neoadjuvant treatment in breast cancer patients. Clin Cancer Res. 2000;6(1):50-6.

23. Gluck S, Ross JS, Royce M, McKenna EF, Jr., Perou CM, Avisar E, et al. TP53 genomics predict higher clinical and pathologic tumor response in operable early-stage breast cancer treated with docetaxel-capecitabine+/−trastuzumab. Breast Cancer Res Treat. 2012;132(3):781-91.

24. Bertheau P, Turpin E, Rickman DS, Espie M, de Reynies A, Feugeas JP, et al. Exquisite sensitivity of TP53 mutant and basal breast cancers to a dose-dense epirubicin-cyclophosphamide regimen. PLoS Med. 2007;4(3):e90.

25. Aas T, Geisler S, Eide GE, Haugen DF, Varhaug JE, Bassoe AM, et al. Predictive value of tumour cell proliferation in locally advanced breast cancer treated with neoadjuvant chemotherapy. Eur J Cancer. 2003 ;39(4): 438-46.

26. Geisler S, Borresen-Dale AL, Johnsen H, Aas T, Geisler J, Akslen LA, et al. TP53 gene mutations predict the response to neoadjuvant treatment with 5-fluorouracil and mitomycin in locally advanced breast cancer. Clin Cancer Res. 2003;9(15):5582-8.

27. Anelli A, Brentani RR, Gadelha AP, Amorim De Albuquerque A, and Soares F. Correlation of p53 status with outcome of neoadjuvant chemotherapy using paclitaxel and doxorubicin in stage IIIB breast cancer. Ann Oncol. 2003;14(3):428-32.

28. Guarneri V, Barbieri E, Piacentini F, Giovannelli S, Ficarra G, Frassoldati A, et al.

Predictive and prognostic role of p53 according to tumor phenotype in breast cancer patients treated with preoperative chemotherapy: a single-institution analysis. Int J Biol Markers. 2010;25(2):104-11.

29. Tiezzi DG, Andrade JM, Ribeiro-Silva A, Zola FE, Marana HR, and Tiezzi MG. HER-2, p53, p21 and hormonal receptors proteins expression as predictive factors of response and prognosis in locally advanced breast cancer treated with neoadjuvant docetaxel plus epirubicin combination. BMC Cancer. 2007;7:36.

30. Bonnefoi H, Piccart M, Bogaerts J, Mauriac L, Fumoleau P, Brain E, et al. TP53 status for prediction of sensitivity to taxane versus non-taxane neoadjuvant chemotherapy in breast cancer (EORTC 10994/BIG 1-00): a randomised phase 3 trial. Lancet Oncol. 2011;12(6):527-39.

31. Darb-Esfahani S, Denkert C, Stenzinger A, Salat C, Sinn B, Schem C, et al. Role of TP53 mutations in triple negative and HER2-positive breast cancer treated with neoadjuvant anthracycline/taxane-based chemotherapy. Oncotarget. 2016;7(42):67686-98.

32. Hurson AN, Abubakar M, Hamilton AM, Conway K, Hoadley KA, Love MI, et al. Prognostic significance of RNA-based TP53 pathway function among estrogen receptor positive and negative breast cancer cases. NPJ Breast Cancer. 2022; 8(1):74.

33. Bakhtiar H, Helzer KT, Park Y, Chen Y, Rydzewski NR, Bootsma ML, et al. Identification of phenocopies improves prediction of targeted therapy response over DNA mutations alone. NPJ Genom Med. 2022;7(1):58.

34. Korde LA, Somerfield MR, Carey LA, Crews JR, Denduluri N, Hwang ES, et al. Neoadjuvant Chemotherapy, Endocrine Therapy, and Targeted Therapy for Breast Cancer: ASCO Guideline. J Clin Oncol. 2021;39(13):1485-505.

35. Cortazar P, Zhang L, Untch M, Mehta K, Costantino JP, Wolmark N, et al. Pathological complete response and long-term clinical benefit in breast cancer: the CTNeoBC pooled analysis. Lancet. 2014;384(9938):164-72.

36. Wolf DM, Yau C, Wulfkuhle J, Brown-Swigart L, Gallagher RI, Lee PRE, et al. Redefining breast cancer subtypes to guide treatment prioritization and maximize response: Predictive biomarkers across 10 cancer therapies. Cancer Cell. 2022;40(6):609-23 e6.

37. Symmans WF, Wei C, Gould R, Yu X, Zhang Y, Liu M, et al. Long-Term Prognostic Risk After Neoadjuvant Chemotherapy Associated With Residual Cancer Burden and Breast Cancer Subtype. J Clin Oncol. 2017;35(10):1049-60.

38. Filho OM, Stover DG, Asad S, Ansell PJ, Watson M, Loibl S, et al. Association of Immunophenotype With Pathologic Complete Response to Neoadjuvant Chemotherapy for Triple-Negative Breast Cancer: A Secondary Analysis of the BrighTNess Phase 3 Randomized Clinical Trial. JAMA Oncol. 2021;7(4):603-8.

39. Magbanua MJ, Wolf DM, Yau C, Davis SE, Crothers J, Au A, et al. Serial expression analysis of breast tumors during neoadjuvant chemotherapy reveals changes in cell cycle and immune pathways associated with recurrence and response. Breast Cancer Res. 2015;17(1):73.

40. Schmid P, Cortes J, Dent R, Pusztai L, McArthur H, Kummel S, et al. Event-free Survival with Pembrolizumab in Early Triple-Negative Breast Cancer. N Engl J Med. 2022;386(6):556-67.

41. Esserman LJ, Berry DA, Cheang MC, Yau C, Perou CM, Carey L, et al. Chemotherapy response and recurrence-free survival in neoadjuvant breast cancer depends on biomarker profiles: results from the I-SPY 1 TRIAL (CALGB 150007/150012; ACRIN 6657). Breast Cancer Res Treat. 2012;132(3):1049-62.

42. Carlsen L, Zhang S, Tian X, De La Cruz A, George A, Arnoff TE, et al. The role of p53 in anti-tumor immunity and response to immunotherapy. Front Mol Biosci. 2023;10:1148389.

43. Dong ZY, Zhong WZ, Zhang XC, Su J, Xie Z, Liu SY, et al. Potential Predictive Value of TP53 and KRAS Mutation Status for Response to PD-1 Blockade Immunotherapy in Lung Adenocarcinoma. Clin Cancer Res. 2017;23(12):3012-24.

44. Malekzadeh P, Yossef R, Cafri G, Paria BC, Lowery FJ, Jafferji M, et al. Antigen Experienced T Cells from Peripheral Blood Recognize p53 Neoantigens. Clin Cancer Res. 2020;26(6):1267-76.

45. Deniger DC, Pasetto A, Robbins PF, Gartner JJ, Prickett TD, Paria BC, et al. T-cell Responses to TP53 “Hotspot” Mutations and Unique Neoantigens Expressed by Human Ovarian Cancers. Clin Cancer Res. 2018;24(22):5562-73.

46. Cree IA, and Charlton P. Molecular chess? Hallmarks of anti-cancer drug resistance. BMC Cancer. 2017; 17(1): 10.

47. Tredan O, Galmarini CM, Patel K, and Tannock IF. Drug resistance and the solid tumor microenvironment. J Natl Cancer Inst. 2007;99(19): 1441 -54.

48. Zhang W, Li E, Wang L, Lehmann BD, and Chen XS. Transcriptome Meta-Analysis of Triple-Negative Breast Cancer Response to Neoadjuvant Chemotherapy. Cancers (Basel). 2023;15(8).

49. Wade M, Li YC, and Wahl GM. MDM2, MDMX and p53 in oncogenesis and cancer therapy. Nat Rev Cancer. 2013 ; 13 (2):83 -96.

50. Aggarwal R, Rydzewski NR, Zhang L, Foye A, Kim W, Helzer KT, et al. Prognosis Associated With Luminal and Basal Subtypes of Metastatic Prostate Cancer. JAMA Oncol. 2021;7(11):1644-52.

1. A method of generating a phenocopy signature, the method comprising:
identifying a gene set comprising one or more genes within a biological pathway;
identifying a set of training cells, wherein each training cell comprises each of the one or more genes in the gene set;
obtaining a nucleic acid sequence for each of the one or more genes in each training cell;
identifying a mutation set comprising one or more mutations within the nucleic acid sequences;
obtaining a gene expression profile for each training cell; and
determining from the mutation set and the gene expression profiles a set of gene expression signatures that predict presence of at least a subset of the one or more mutations within the training cells, wherein the phenocopy signature comprises the set of gene expression signatures.

2. The method of claim 1, wherein the biological pathway is selected from the group consisting of a cell cycle pathway, a DNA repair pathway, a metabolism pathway, a signaling pathway (e.g., signal transduction by a receptor (e.g., a growth factor receptor) and second messengers), a transcriptional regulation pathway, a transport pathway (e.g., of transmembrane transporters), a cell motility pathway, an immune function pathway, a cell death pathway, a host-virus interaction pathway, a cellular stress-response pathway, a developmental pathway, a senescence pathway, an angiogenesis pathway, an epithelial-to-mesenchymal transition pathway, and a neural pathway.

3. The method of claim 1, wherein the biological pathway is a disease pathway.

4. The method of claim 1, wherein the biological pathway is a cancer pathway.

5. The method of claim 1, wherein at least one of the one or more mutations in the mutation set is a pathogenic mutation.

6. The method of claim 1, wherein each of the one or more mutations in the mutation set is a pathogenic mutation.

7. The method of claim 1, wherein at least one of the one or more mutations in the mutation set is not known to be pathogenic.

8. The method of claim 1, wherein at least one of the one or more mutations in the mutation set is a coding mutation.

9. The method of claim 1, wherein each of the one or more mutations in the mutation set is a coding mutation.

10. The method of claim 1, wherein at least one or more of the training cells comprises a pathological cell.

11. The method of claim 1, wherein each of the training cells is a pathological cell.

12. The method of claim 1, wherein the determining the phenocopy signature does not comprise incorporating empirical drug-response data for at least one of the training cells.

13. The method of claim 1, wherein the determining the phenocopy signature does not comprise incorporating empirical drug-response data for any of the training cells.

14. The method of claim 1, wherein the obtaining the nucleic acid sequence for each of the one or more genes in each training cell comprises sequencing at least a portion of at least one of the one or more genes in at least one training cell.

15. (canceled)

16. The method of claim 1, wherein the obtaining the gene expression profile for each training cell comprises measuring the mRNA levels of different mRNA species present in at least one training cell.

17. The method of claim 1, wherein the obtaining the gene expression profile for each training cell comprises sequencing at least a portion of each of the one or more genes in each training cell or measuring the mRNA levels of different mRNA species present in each training cell.

18. A method of identifying a cell exhibiting the phenocopy signature of claim 1, the method comprising:
obtaining a gene expression profile for a test cell; and
determining whether the gene expression profile for the test cell matches the phenocopy signature, wherein the test cell exhibits the phenocopy signature if the gene expression profile for the test cell matches the phenocopy signature.

19-20. (canceled)

21. A method of identifying a subject comprising cells that exhibit the phenocopy signature of claim 1, the method comprising:
isolating a test cell from the subject;
obtaining a gene expression profile for the test cell; and
determining whether the gene expression profile for the test cell matches the phenocopy signature, wherein the test cell exhibits the phenocopy signature if the gene expression profile for the test cell matches the phenocopy signature.

22-23. (canceled)

24. A method of predicting a subject sensitive to treatment with a drug that targets a biological pathway and, optionally, treating the subject, the method comprising:
obtaining a gene expression profile for a test cell isolated from the subject; and
determining whether the gene expression profile for the test cell matches a phenocopy signature generated with a gene set from the biological pathway that the drug targets, wherein the subject is predicted to be sensitive to treatment with the drug if the gene expression profile for the test cell matches the phenocopy signature, wherein the phenocopy signature and the biological pathway are the phenocopy signature and the biological pathway, respectively, of claim 1.

25-34. (canceled)

35. A method of predicting a cell sensitive to treatment with a drug that targets a biological pathway and, optionally, treating the cell, the method comprising:
obtaining a gene expression profile for a test cell; and
determining whether the gene expression profile for the test cell matches a phenocopy signature generated with a gene set from the biological pathway that the drug targets, wherein the cell is predicted to be sensitive to treatment with the drug if the gene expression profile for the test cell matches the phenocopy signature,
wherein the phenocopy signature and the biological pathway are the phenocopy signature and the biological pathway, respectively, of claim 1.

36-42. (canceled)
